(19) United States
(12) Patent Application Publication
(10) Pub. No.: US 2026/0035717 A1
(43) Pub. Date: Feb. 5, 2026

(54) ADENO-ASSOCIATED VIRAL VECTOR COMPOSITIONS AND METHODS OF USE

(71) Applicant: Siren Biotechnology, Inc., San Francisco, CA (US)

(72) Inventors: Nicole Kristen Paulk, San Francisco, CA (US); Nathalie Andree Nancy Clement, Gainesville, FL (US); Siti Nur Sarah Morris, Vallejo, CA (US)

(21) Appl. No.: 19/361,281

(22) Filed: Oct. 17, 2025

Related U.S. Application Data
(63) Continuation of application No. PCT/US2024/025194, filed on Apr. 18, 2024.
(60) Provisional application No. 63/496,916, filed on Apr. 18, 2023.
(60) Provisional application No. 63/502,546, filed on May 16, 2023.

(51) Int. Cl. C12N 15/86; A61K 38/21; A61K 48/00; A61P 35/00; C07K 14/565

(52) U.S. Cl. CPC C12N 15/86; A61K 38/215; A61K 48/005; A61P 35/00; C07K 14/565; C12N 2750/14143; C12N 2830/48; C12N 2830/50

(57) ABSTRACT

Described herein are nucleic acids and recombinant viral vectors comprising said nucleic acids, wherein said nucleic acids encode a payload of interest (e.g., an interferon).

### CROSS REFERENCE TO RELATED APPLICATIONS

This application is a continuation of International Application No. PCT/US2024/025194, filed Apr. 18, 2024, which claims the benefit of and priority to U.S. Provisional Application Nos. 63/496,916 and 63/502,546 filed Apr. 18, 2023, and May 16, 2023, respectively, the entire disclosures of each of which are hereby incorporated by reference in their entirety for all purposes.

### SEQUENCE LISTING

This application contains a sequence listing in XML format. The contents of the electronic sequence listing (File Name: 00018.004.1301_SL.xml; Size: 104,245 bytes; and Date of Creation: Oct. 17, 2025) is herein incorporate by reference in its entirety.

### SUMMARY OF THE DISCLOSURE

Described herein, in certain embodiments, are recombinant adeno-associated viral (rAAV) vectors for use in delivering a payload of interest (e.g., a therapeutic payload) to a subject in need thereof and which exhibit improved efficacy and/or reduced immunogenicity.

Further described herein, in certain embodiments, are rAAV vectors comprising a coding sequence for a payload of interest (e.g., a therapeutic polypeptide) where CpG dinucleotides within said coding sequence have been reduced, depleted, and/or methylated, thereby producing certain beneficial therapeutic characteristics. In some embodiments, a coding sequence that comprises one or more modified CpG dinucleotides (e.g., CpG dinucleotide reduction, CpG dinucleotide depletion and/or CpG dinucleotide methylation), as described herein, encodes a therapeutic polypeptide. In some embodiments, a coding sequence that comprises one or more modified CpG dinucleotides, as described herein, encodes an interferon (e.g., any interferon known in the art, e.g., those described herein).

Also described herein, in certain embodiments, are methods of treating a disease or disorder in a subject in need thereof comprising administering a modified rAAV vector as described herein. In some embodiments, the present disclosure provides for methods of treating a cancer in a subject that include administering a modified rAAV vector as described herein. In some embodiments, a cancer is a brain cancer. In some embodiments, a cancer is an eye cancer. In some embodiments, a cancer is a primary cancer. In some embodiments, a cancer is a metastatic cancer. In some embodiments, a cancer is a glioma, such as a grade III or grade IV glioma (glioblastoma). In some embodiments, a cancer is a glioblastoma. In some embodiments, a cancer is a high-grade glioma, metastatic brain tumor, or uveal melanoma. In some embodiments, administration of a rAAV vector comprising a cytokine (e.g., any cytokine delivering rAAV vector as described herein) is local to a tumor in a subject with cancer. In some embodiments, administration of a rAAV vector comprising a cytokine to a subject with cancer is systemic (e.g., intravenous). Further, rAAV vector compositions and methods set forth herein provide long-term cytokine production (e.g., through vectorization, robust expression, and so forth), high-specificity for payload delivery (e.g., through localized administration), and minimal risk for systemic toxicity (e.g., through low-dose administration of self-limiting AAV genomes).

Described herein, in certain embodiments, are recombinant adeno-associated viral (rAAV) vector comprising, from 5′ to 3′: a) a CAG promoter; and b) a polynucleotide encoding a payload of interest, the polynucleotide comprising reduced CpG dinucleotides and/or increased methylation of CpG dinucleotides as compared to a parental equivalent.

Described herein, in certain embodiments, are recombinant adeno-associated viral (rAAV) vector comprising, from 5′ to 3′: a) a CAG promoter; b) a minigene comprising a splice modulator binding site; c) a polynucleotide encoding a payload of interest, the polynucleotide comprising reduced CpG dinucleotides and/or increased methylation of CpG dinucleotides as compared to a parental equivalent.

In some embodiments, the payload of interest is a therapeutic polypeptide. In some embodiments, the therapeutic polypeptide is a cytokine or interleukin. In some embodiments, the therapeutic polypeptide is a cytokine. In some embodiments, the cytokine is a colony stimulating factor (CSF), a transforming growth factor, a tumor necrosis factor, an interleukin, or an interferon. In some embodiments, the cytokine is an interferon. In some embodiments, methylation of the CpG nucleotides is increased by at least about 5%, 10%, 15%, 20%, 25%, 30%, 35%, 40%, 45%, 50%, 55%, 60%, 65%, 70%, 75%, 80%, 85%, 90%, 95%, 99%, or more as compared to a parental equivalent. In some embodiments, the CpG dinucleotides are completely methylated. In some embodiments, the CpG dinucleotides are reduced by at least about 5%, 10%, 15%, 20%, 25%, 30%, 35%, 40%, 45%, 50%, 55%, 60%, 65%, 70%, 75%, 80%, 85%, 90%, 95%, 99%, or more as compared to a parental equivalent. In some embodiments, the CpG dinucleotides are depleted.

Described herein, in certain embodiments, are recombinant adeno-associated viral (rAAV) vector comprising, from 5′ to 3′: a) a CAG promoter; b) a polynucleotide encoding an interferon; and c) a polynucleotide comprising a WPRE comprising SEQ ID NO: 33.

In some embodiments, the CAG promoter comprises a cytomegalovirus (CMV) immediate early enhancer element, a promoter element, and a splice acceptor element. In some embodiments, the CMV early enhancer element is derived from a wild-type CMV enhancer. In some embodiments, the CMV immediate early enhancer element is truncated with respect to a wild-type CMV enhancer. In some embodiments, the CMV immediate early enhancer element comprises a polynucleotide with at least 80%, at least 85%, at least 90%, at least 95%, at least 96%, at least 97%, at least 98%, or at least 99% sequence identity to SEQ ID NO: 25. In some embodiments, the CMV immediate early enhancer element comprises a polynucleotide with at least 80% sequence identity to SEQ ID NO: 26. In some embodiments, the CMV immediate early enhancer element comprises a polynucleotide with at least 90% sequence identity to SEQ ID NO: 26. In some embodiments, the CMV immediate early enhancer element comprises a polynucleotide sequence as set forth in SEQ ID NO: 26. In some embodiments, the CMV early enhancer element comprises a polynucleotide with at least 80% sequence identity to SEQ ID NO: 27. In some embodiments, the CMV immediate early enhancer element comprises a polynucleotide with at least 90% sequence identity to SEQ ID NO: 27. In some embodiments, the CMV immediate early enhancer element comprises a polynucleotide sequence as set forth in SEQ ID NO: 27. In some embodiments, the promoter element is derived from a chicken beta-actin gene. In some embodiments, the promoter element comprises a polynucleotide with at least 80% sequence identity to SEQ ID NO: 28. In some embodiments, the promoter element comprises a polynucleotide with at least 80% sequence identity to SEQ ID NO: 52. In some embodiments, the promoter element comprises a polynucleotide with at least 90% sequence identity to SEQ ID NO: 28. In some embodiments, the promoter element comprises a polynucleotide with at least 90% sequence identity to SEQ ID NO: 52. In some embodiments, the promoter element comprises a polynucleotide sequence as set forth in SEQ ID NO: 28. In some embodiments, the promoter element comprises a polynucleotide sequence as set forth in SEQ ID NO: 52. In some embodiments, the splice acceptor is derived from a rabbit beta-globin gene. In some embodiments, the splice acceptor comprises a polynucleotide with at least 80% sequence identity to SEQ ID NO: 30. In some embodiments, the splice acceptor comprises a polynucleotide with at least 80% sequence identity to SEQ ID NO: 53. In some embodiments, the splice acceptor comprises a polynucleotide with at least 90% sequence identity to SEQ ID NO: 30. In some embodiments, the splice acceptor comprises a polynucleotide with at least 90% sequence identity to SEQ ID NO: 53. In some embodiments, the splice acceptor comprises a polynucleotide sequence as set forth in SEQ ID NO: 30. In some embodiments, the splice acceptor comprises a polynucleotide sequence as set forth in SEQ ID NO: 53. In some embodiments, the interleukin is IL-1 alpha, IL-1 beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, or IL-36. In some embodiments, the CSF is CSF1 (M-CSF), CSF2 (GM-CSF), or CSF3 (G-CSF). In some embodiments, the TGF is TGF-β1, TGF-β2, or TGF-β3. In some embodiments, the TNF is TNF-α, TNF-β, or LT-β. In some embodiments, the interferon is IFNα, IFNβ, IFNγ, IFNε, IFNκ, IFNω, IFNλ, or variants or derivatives thereof. In some embodiments, the interferon is a human IFNα, IFNβ, IFNγ, IFNε, IFNκ, IFNω, IFNλ, or variants or derivatives thereof. In some embodiments, the interferon is a human IFNβ. In some embodiments, the human IFNβ comprises an amino acid sequence with at least 80% sequence identity to SEQ ID NO: 1. In some embodiments, the human IFNβ comprises an amino acid with at least 90% sequence identity to SEQ ID NO: 1. In some embodiments, the human IFNβ comprises an amino acid sequence as set forth in SEQ ID NO: 1. In some embodiments, the human IFNβ is encoded by a polynucleotide with at least 80% sequence identity to SEQ ID NO: 2, SEQ ID NO: 3, or SEQ ID NO: 4. In some embodiments, the human IFNβ is encoded by a polynucleotide with at least 90% sequence identity to SEQ ID NO: 2, SEQ ID NO: 3, or SEQ ID NO: 4. In some embodiments, the human IFNβ is encoded by a polynucleotide as set forth in SEQ ID NO: 2, SEQ ID NO: 3, or SEQ ID NO: 4. In some embodiments, the interferon is a human IFNα. In some embodiments, the human IFNα comprises an amino acid sequence with at least 80% sequence identity to SEQ ID NO: 5. In some embodiments, the human IFNα comprises an amino acid sequence with at least 90% sequence identity to SEQ ID NO: 5. In some embodiments, the human IFNα comprises an amino acid sequence as set forth in SEQ ID NO: 5. In some embodiments, the human IFNα is encoded by a polynucleotide with at least 80% sequence identity to SEQ ID NO: 6, SEQ ID NO: 7, or SEQ ID NO: 8. In some embodiments, the human IFNα is encoded by a polynucleotide with at least 90% sequence identity to SEQ ID NO: 6, SEQ ID NO: 7, or SEQ ID NO: 8. In some embodiments, the human IFNα is encoded by a polynucleotide as set forth in SEQ ID NO: 6, SEQ ID NO: 7, or SEQ ID NO: 8. In some embodiments, the interferon is a human IFNγ. In some embodiments, the human IFNγ comprises an amino acid sequence with at least 80% sequence identity to SEQ ID NO: 9. In some embodiments, the human IFNγ comprises an amino acid sequence with at least 90% sequence identity to SEQ ID NO: 9. In some embodiments, the human IFNγ comprises an amino acid sequence as set forth in SEQ ID NO: 9. In some embodiments, the human IFNγ is encoded by a polynucleotide with at least 80% sequence identity to SEQ ID NO: 10, SEQ ID NO: 11, or SEQ ID NO: 12. In some embodiments, the human IFNγ is encoded by a polynucleotide with at least 90% sequence identity to SEQ ID NO: 10, SEQ ID NO: 11, or SEQ ID NO: 12. In some embodiments, the human IFNγ is encoded by a polynucleotide sequence as set forth in SEQ ID NO: 10, SEQ ID NO: 11, or SEQ ID NO: 12. In some embodiments, the interferon is a mouse IFNα, IFNβ, IFNγ, IFNε, IFNκ, IFNω, IFNλ, or variants or derivatives thereof. In some embodiments, the interferon is a mouse IFNβ. In some embodiments, the mouse IFNβ comprises an amino acid sequence with at least 80% sequence identity to SEQ ID NO: 13. In some embodiments, the mouse IFNβ comprises an amino acid sequence with at least 90% sequence identity to SEQ ID NO: 13. In some embodiments, the mouse IFNβ comprises an amino acid sequence as set forth in SEQ ID NO: 13. In some embodiments, the mouse IFNβ is encoded by a polynucleotide with at least 80% sequence identity to SEQ ID NO: 14. In some embodiments, the mouse IFNβ is encoded by a polynucleotide with at least 90% sequence identity to SEQ ID NO: 14. In some embodiments, the mouse IFNβ is encoded by a polynucleotide as set forth in SEQ ID NO: 14. In some embodiments, the mouse IFNβ is encoded by a polynucleotide with at least 80% sequence identity to SEQ ID NO: 42. In some embodiments, the mouse IFNβ is encoded by a polynucleotide with at least 90% sequence identity to SEQ ID NO: 42. In some embodiments, the mouse IFNβ is encoded by a polynucleotide as set forth in SEQ ID NO: 42. In some embodiments, the interferon is a canine IFNα, IFNβ, IFNγ, IFNε, IFNκ, IFNω, IFNλ, or variants or derivatives thereof. In some embodiments, the interferon is a canine IFNβ. In some embodiments, the canine IFNβ comprises an amino acid sequence with at least 80% sequence identity to SEQ ID NO: 15. In some embodiments, the canine IFNβ comprises an amino acid sequence with at least 90% sequence identity to SEQ ID NO: 15. In some embodiments, the canine IFNβ comprises an amino acid sequence as set forth in SEQ ID NO: 15. In some embodiments, the canine IFNβ is encoded by a polynucleotide with at least 80% sequence identity to SEQ ID NO: 16. In some embodiments, the canine IFNβ is encoded by a polynucleotide with at least 90% sequence identity to SEQ ID NO: 16. In some embodiments, the canine IFNβ is encoded by a polynucleotide as set forth in SEQ ID NO: 16. In some embodiments, the canine IFNβ is encoded by a polynucleotide with at least 80% sequence identity to SEQ ID NO: 43. In some embodiments, the canine IFNβ is encoded by a polynucleotide with at least 90% sequence identity to SEQ ID NO: 43. In some embodiments, the canine IFNβ is encoded by a polynucleotide as set forth in SEQ ID NO: 43. In some embodiments, the canine IFNβ is encoded by a polynucleotide with at least 80% sequence identity to SEQ ID NO: 60. In some embodiments, the canine IFNβ is encoded by a polynucleotide with at least 90% sequence identity to SEQ ID NO: 60. In some embodiments, the canine IFNβ is encoded by a polynucleotide as set forth in SEQ ID NO: 60. In some embodiments, the interferon is a rat IFNα, IFNβ, IFNγ, IFNε, IFNκ, IFNω, IFNλ, or variants or derivatives thereof. In some embodiments, the interferon is a rat IFNβ. In some embodiments, the rat IFNβ comprises an amino acid sequence with at least 80% sequence identity to SEQ ID NO: 61. In some embodiments, the rat IFNβ comprises an amino acid sequence with at least 90% sequence identity to SEQ ID NO: 61. In some embodiments, the rat IFNβ comprises an amino acid sequence as set forth in SEQ ID NO: 61. In some embodiments, the rat IFNβ is encoded by a polynucleotide with at least 80% sequence identity to SEQ ID NO: 62. In some embodiments, the rat IFNβ is encoded by a polynucleotide with at least 90% sequence identity to SEQ ID NO: 62. In some embodiments, the rat IFNβ is encoded by a polynucleotide as set forth in SEQ ID NO: 62. In some embodiments, the rat IFNβ is encoded by a polynucleotide with at least 80% sequence identity to SEQ ID NO: 63. In some embodiments, the rat IFNβ is encoded by a polynucleotide with at least 90% sequence identity to SEQ ID NO: 63. In some embodiments, the rat IFNβ is encoded by a polynucleotide as set forth in SEQ ID NO: 63. In some embodiments, the interferon is a guinea pig IFNα, IFNβ, IFNγ, IFNε, IFNκ, IFNω, IFNλ, or variants or derivatives thereof. In some embodiments, the interferon is a guinea pig IFNβ. In some embodiments, the guinea pig IFNβ comprises an amino acid sequence with at least 80% sequence identity to SEQ ID NO: 71. In some embodiments, the guinea pig IFNβ comprises an amino acid sequence with at least 90% sequence identity to SEQ ID NO: 71. In some embodiments, the guinea pig IFNβ comprises an amino acid sequence as set forth in SEQ ID NO: 71. In some embodiments, the guinea pig IFNβ is encoded by a polynucleotide with at least 80% sequence identity to SEQ ID NO: 72. In some embodiments, the guinea pig IFNβ is encoded by a polynucleotide with at least 90% sequence identity to SEQ ID NO: 72. In some embodiments, the guinea pig IFNβ is encoded by a polynucleotide as set forth in SEQ ID NO: 72. In some embodiments, the interferon coding polynucleotide comprises reduced CpG dinucleotides and/or increased methylation of CpG dinucleotides as compared to a parental equivalent. In some embodiments, methylation of the CpG nucleotides is increased by at least about 5%, 10%, 15%, 20%, 25%, 30%, 35%, 40%, 45%, 50%, 55%, 60%, 65%, 70%, 75%, 80%, 85%, 90%, 95%, or more than 95% as compared to a parental equivalent. In some embodiments, the CpG dinucleotides are completely methylated. In some embodiments, the CpG dinucleotides are reduced by at least about 5%, 10%, 15%, 20%, 25%, 30%, 35%, 40%, 45%, 50%, 55%, 60%, 65%, 70%, 75%, 80%, 85%, 90%, 95%, or more than 99% as compared to a parental equivalent. In some embodiments, the CpG dinucleotides are depleted. In some embodiments, the rAAV vector further comprises a first ITR sequence and a second ITR sequence. In some embodiments, the rAAV vector comprises, from 5′ to 3′: a) the first ITR sequence; b) the promoter; c) the polynucleotide encoding a payload of interest; and d) the second ITR sequence. In some embodiments, the rAAV vector comprises, from 5′ to 3′: a) the first ITR sequence; b) the promoter; c) the minigene; d) the polynucleotide encoding a payload of interest; and e) the second ITR sequence. In some embodiments, the first ITR sequence and/or the second ITR sequence are truncated as compared to their correspondence wild-type ITR sequence. In some embodiments, the first ITR sequence and/or the second ITR sequence are truncated by at least about 5 nucleotides at the 5′ end or 3′ end. In some embodiments, the first ITR sequence is truncated by 20 nucleotides at the 5′ end. In some embodiments, the second ITR sequence is truncated by 20 nucleotides at the 3′ end. In some embodiments, the first ITR sequence and/or the second ITR sequence comprise an ITR sequence derived from an AAV serotype selected from the group consisting of: AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof. In some embodiments, the first ITR sequence and/or the second ITR sequence are, or are derived from, an AAV2 ITR sequence. In some embodiments, the first ITR sequence and/or the second ITR sequence comprise a polynucleotide with at least 80% sequence identity to SEQ ID NO: 36-41. In some embodiments, the first ITR sequence and/or the second ITR sequence comprise a polynucleotide with at least 90% sequence identity to SEQ ID NO: 36-41. In some embodiments, the first ITR sequence and/or the second ITR sequence comprise a polynucleotide sequence as set forth in SEQ ID NO: 36-41. In some embodiments, the rAAV vector further comprises at least one regulatory element. In some embodiments, the regulatory element is selected from the group consisting of: a promoter, an enhancer, a terminator sequence, an mRNA stability sequence, a sequence that allows for internal ribosome entry sites (IRES) of bicistronic mRNA, an intron, a synthetic intron, a sequence that inhibits viral recognition, a sequence necessary for transduction into a cell, and a polyA sequence. In some embodiments, the regulatory element is a promoter. In some embodiments, the promoter is selected from the group consisting of: a mini promoter, an inducible promoter, a constitutive promoter, and derivatives thereof. In some embodiments, the promoter is selected from the group consisting of: CMV, CBA, EF1a, CAG, PGK, TRE, U6, UAS, T7, Sp6, lac, araBad, trp, Ptac, p5, p19, p40, Synapsin, GFAP, CaMKII, GRK1, and derivatives thereof. In some embodiments, the promoter is a CAG promoter. In some embodiments, the sequence that allows for internal ribosome entry sites (IRES) of bicistronic mRNA is a WPRE. In some embodiments, the WPRE is a wild-type WPRE. In some embodiments, the WPRE comprises a polynucleotide with at least 80% sequence identity to SEQ ID NO: 32. In some embodiments, the WPRE comprises a polynucleotide with at least 90% sequence identity to SEQ ID NO: 32. In some embodiments, the WPRE comprises a polynucleotide as set forth in SEQ ID NO: 32. In some embodiments, the WPRE is a modified WPRE. In some embodiments, the modified WPRE comprises a polynucleotide with at least 80% sequence identity to SEQ ID NO: 33. In some embodiments, the modified WPRE comprises an amino acid sequence with at least 90% sequence identity to SEQ ID NO: 33. In some embodiments, the modified WPRE comprises an amino acid sequence as set forth in SEQ ID NO: 33. In some embodiments, the polyA sequence is selected from the group consisting of: a SV40, hGH, bGH, and rbGlob. In some embodiments, the poly A sequence is a SV40 sequence. In some embodiments, the SV40 sequence comprises a polynucleotide with at least 80% sequence identity to SEQ ID NO: 19. In some embodiments, the SV40 sequence comprises a polynucleotide with at least 90% sequence identity to SEQ ID NO: 19. In some embodiments, the SV40 sequence comprises a polynucleotide as set forth in SEQ ID NO: 19. In some embodiments, the polyA sequence is a bGH sequence. In some embodiments, the bGH sequence comprises a polynucleotide with at least 80% sequence identity to SEQ ID NO: 34. In some embodiments, the bGH sequence comprises a polynucleotide with at least 90% sequence identity to SEQ ID NO: 34. In some embodiments, the bGH sequence comprises a polynucleotide sequence as set forth in SEQ ID NO: 34. In some embodiments, the rAAV vector further comprises a minigene 5′ to the polynucleotide encoding the payload of interest. In some embodiments, the minigene encodes a splice modulator binding site. In some embodiments, the splice modulator binding site is located in an exon and/or an intron. In some embodiments, the splice modulator binding site comprises one or more sequences required for spliceosome binding. In some embodiments, the splice modulator binding site comprises a donor site sequence, a branch site, and an acceptor site. In some embodiments, the minigene encodes an in-frame translation stop codon. In some embodiments, the polynucleotide encoding a payload of interest further comprises a translation stop codon. In some embodiments, the polynucleotide encoding a payload of interest does not comprise a start codon. In some embodiments, the polynucleotide encoding a payload of interest does not comprise an in-frame open reading frame. In some embodiments, the minigene is regulated by a small molecule splicing modifier. In some embodiments, the small molecule splicing modifier is sudemycin, LMI070, RG7916, or RG7800. In some embodiments, the small molecule splicing modifier is selected from the group consisting of:

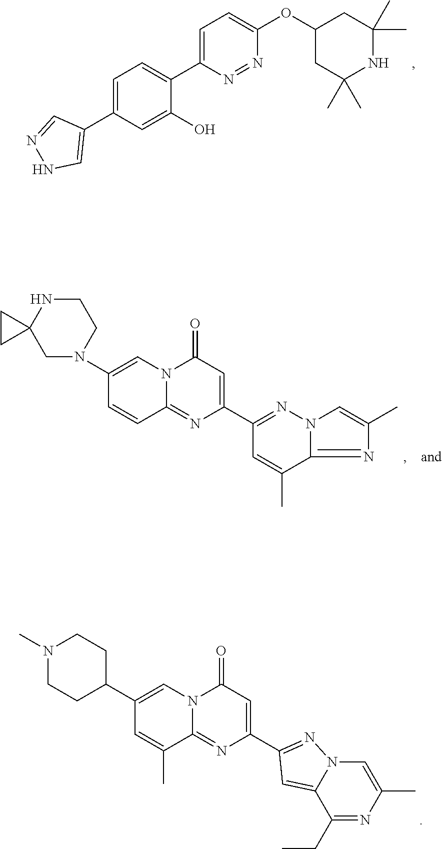

In some embodiments, the minigene comprises exons 6, 7, and 8 of SMN2 and the splice modulator binding site is recognized by LMI070. In some embodiments, the minigene comprises at least about 80% sequence identity to SEQ ID NO: 31. In some embodiments, the minigene comprises at least about 90% sequence identity to SEQ ID NO: 31. In some embodiments, the minigene comprises a polynucleotide as set forth in SEQ ID NO: 31. In some embodiments, the minigene comprises at least about 80% sequence identity to SEQ ID NO: 51. In some embodiments, the minigene comprises at least about 90% sequence identity to SEQ ID NO: 51. In some embodiments, the minigene comprises a polynucleotide as set forth in SEQ ID NO: 51. In some embodiments, the minigene is regulated by a disease state in a cell. In some embodiments, the disease state is cancer. In some embodiments, the cancer is glioblastoma, metastatic brain tumor, or uveal melanoma. In some embodiments, the minigene is regulated by a cell type or tissue type. In some embodiments, the polynucleotide comprising the minigene and the polynucleotide encoding the payload of interest are linked by a polynucleotide sequence encoding a cleavable peptide. In some embodiments, the cleavable peptide is a self-cleaving peptide, a drug-sensitive protease, or a substrate for an endogenous endoprotease. In some embodiments, the rAAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof. In some embodiments, the rAAV vectors further comprise an antibiotic resistance gene. In some embodiments, the antibiotic resistance gene confers resistance to an aminoglycoside, a beta-lactam, a macrolide, a tetracycline, or a derivative thereof. In some embodiments, the antibiotic resistance gene confers resistance to kanamycin, spectinomycin, streptomycin, ampicillin, carbenicillin, bleomycin, erythromycin, polymyxin B, tetracycline, chloramphenicol, neomycin, zeocin, or a derivative thereof. In some embodiments, the antibiotic resistance gene confers resistance to kanamycin. In some embodiments, the antibiotic resistance gene comprises a nucleic acid sequence having at least about 80% sequence identity to SEQ ID NO: 20, SEQ ID NO: 21, SEQ ID NO: 44, or SEQ ID NO: 55. In some embodiments, the antibiotic resistance gene comprises a nucleic acid sequence having at least about 90% sequence identity to SEQ ID NO: 20, SEQ ID NO: 21, SEQ ID NO: 44, or SEQ ID NO: 55. In some embodiments, the antibiotic resistance gene comprises a nucleic acid as set forth in SEQ ID NO: 20, SEQ ID NO: 21, SEQ ID NO: 44, or SEQ ID NO: 55. In some embodiments, the antibiotic resistance gene comprises reduced CpG dinucleotides and/or increased methylation of CpG dinucleotides as compared to a parental equivalent. In some embodiments, methylation of the CpG nucleotides is increased by at least about 5%, 10%, 15%, 20%, 25%, 30%, 35%, 40%, 45%, 50%, 55%, 60%, 65%, 70%, 75%, 80%, 85%, 90%, 95%, or more than 95% as compared to a parental equivalent. In some embodiments, the CpG dinucleotides are completely methylated. In some embodiments, the CpG dinucleotides are reduced by at least about 5%, 10%, 15%, 20%, 25%, 30%, 35%, 40%, 45%, 50%, 55%, 60%, 65%, 70%, 75%, 80%, 85%, 90%, 95%, or more than 99% as compared to a parental equivalent. In some embodiments, the CpG dinucleotides are depleted. In some embodiments, the rAAV vector further comprises an origin of replication. In some embodiments, the origin of replication is selected from the group consisting of: pMB1, pBR322, ColE1, R6K, p15A, pSC101, ColE2, F1, pUC, pBluescript, and combinations or a derivative thereof.

Described herein, in certain embodiments, are methods of treating cancer in a subject in need thereof comprising: a) administering the rAAV vector as described herein; and b) administering a small molecule splicing modifier. In some embodiments, the subject is human. In some embodiments, the administration is to the central nervous system. In some embodiments, the administration is to a brain. In some embodiments, the administration is to a brain ventricle. In some embodiments, the administration is by Convection Enhanced Delivery (CED). In some embodiments, the administration is by intratumoral injection, intracranial injection, intracerebral injection, intracerebroventricular, intraparenchymal, or injection into the cerebrospinal fluid (CSF) via the cerebral ventricular system, cisterna magna, or intrathecal space. In some embodiments, the small molecule splicing modifier is sudemycin, LMI070, RG7916, or RG7800. In some embodiments, the small molecule splicing modifier is selected from the group consisting of:

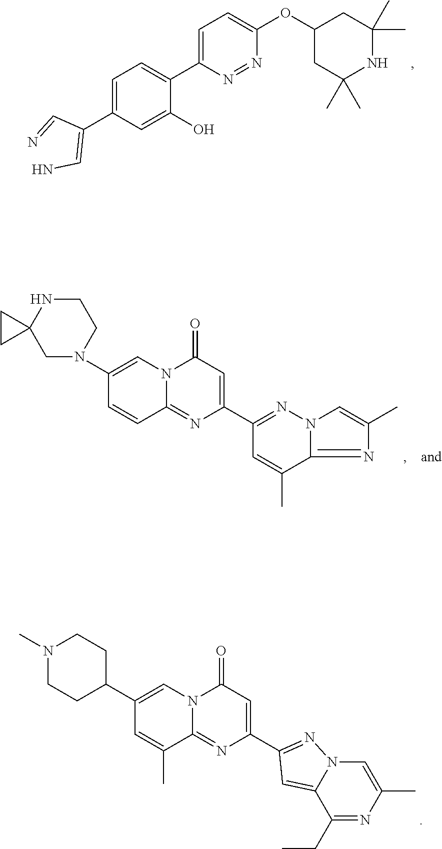

Described herein, in certain embodiments, are recombinant adeno-associated viral (rAAV) vectors comprising, from 5′ to 3′: a) a first ITR as set forth in SEQ ID NO: 40 or 41; b) a CAG promoter as set forth in any one of SEQ ID NOs: 22-23, 48-50, and 64-65; c) a polynucleotide encoding a CpG-depleted human interferon as set forth in SEQ ID NO: 4; d) a WPRE sequence as set forth in SEQ ID NO: 33; e) a bGH polyA as set forth in SEQ ID NO: 34; and f) a second ITR as set forth in SEQ ID NO: 40 or 41.

Described herein, in certain embodiments, are recombinant adeno-associated viral (rAAV) vectors comprising, from 5′ to 3′: a) a first ITR as set forth in SEQ ID NO: 40 or 41; b) a CAG promoter as set forth in any one of SEQ ID NOs: 22-23, 48-50, and 64-65; c) a polynucleotide encoding a CpG-depleted human interferon as set forth in SEQ ID NO: 4; d) a WPRE sequence as set forth in SEQ ID NO: 33; e) a SV40 polyA as set forth in SEQ ID NO: 19; and f) a second ITR as set forth in SEQ ID NO: 40 or 41.

Described herein, in certain embodiments, are recombinant adeno-associated viral (rAAV) vectors comprising, from 5′ to 3′: a) a first ITR comprising as set forth in any one of SEQ ID NOs: 36-39; b) a CAG promoter as set forth in any one of SEQ ID NOs: 22-23, 48-50, and 64-65; c) a polynucleotide encoding a CpG-depleted human interferon as set forth in SEQ ID NO: 4; d) a WPRE sequence as set forth in SEQ ID NO: 33; e) a bGH polyA as set forth in SEQ ID NO: 34; and f) a second ITR sequence as set forth in any one of SEQ ID NOs: 36-39.

Described herein, in certain embodiments, are recombinant adeno-associated viral (rAAV) vectors comprising, from 5′ to 3′: a) a first ITR as set forth in SEQ ID NO: 40 or 41; b) a CAG promoter as set forth in any one of SEQ ID NOs: 22-23, 48-50, and 64-65; c) a polynucleotide encoding a human interferon as set forth in SEQ ID NO: 2 or SEQ ID NO: 3; d) a WPRE sequence as set forth in SEQ ID NO: 33; e) a SV40 polyA as set forth in SEQ ID NO: 19; and f) a second ITR as set forth in SEQ ID NO: 40 or 41.

Described herein, in certain embodiments, are recombinant adeno-associated viral (rAAV) vectors comprising, from 5′ to 3′: a) a first ITR as set forth in SEQ ID NO: 40 or 41; b) a CAG promoter as set forth in any one of SEQ ID NOs: 22-23, 48-50, and 64-65; c) a minigene as set forth in SEQ ID NO: 31 or 51; d) a polynucleotide encoding a CpG-depleted human interferon as set forth in SEQ ID NO: 4; e) a WPRE sequence as set forth in SEQ ID NO: 33; f) a SV40 polyA as set forth in SEQ ID NO: 19; and g) a second ITR as set forth in SEQ ID NO: 40 or 41.

Described herein, in certain embodiments, are recombinant adeno-associated viral (rAAV) vectors comprising, from 5′ to 3′: a) a first ITR as set forth in SEQ ID NO: 40 or 41; b) a CAG promoter as set forth in any one of SEQ ID NOs: 22-23, 48-50, and 64-65; c) a minigene as set forth in SEQ ID NO: 31; d) a polynucleotide encoding a CpG-depleted human interferon as set forth in SEQ ID NO: 4; e) a WPRE sequence as set forth in SEQ ID NO: 33; f) a bGH polyA as set forth in SEQ ID NO: 34; and g) a second ITR as set forth in SEQ ID NO: 40 or 41.

Described herein, in certain embodiments, are recombinant adeno-associated viral (rAAV) vectors comprising, from 5′ to 3′: a) a first ITR as set forth in SEQ ID NO: 36-39; b) a CAG promoter as set forth in any one of SEQ ID NOs: 22-23, 48-50, and 64-65; c) a minigene as set forth in SEQ ID NO: 31 or 51; d) a polynucleotide encoding a CpG-depleted human interferon as set forth in SEQ ID NO: 4; e) a WPRE sequence as set forth in SEQ ID NO: 33; f) a bGH polyA as set forth in SEQ ID NO: 34; and g) a second ITR as set forth in SEQ ID NO: 36-39.

Described herein, in certain embodiments, are recombinant adeno-associated viral (rAAV) vectors comprising, from 5′ to 3′: a) a first ITR sequence; b) a CAG promoter; c) a coding sequence for a CpG-depleted human interferon; d) a WPRE sequence; e) a SV40 polyA sequence; and f) a second ITR sequence.

Described herein, in certain embodiments, are recombinant adeno-associated viral (rAAV) vectors comprising, from 5′ to 3′: a) a first ITR sequence; b) a CAG promoter; c) a coding sequence for a CpG-depleted human interferon; d) a WPRE sequence; e) a bGH polyA sequence; and f) a second ITR sequence.

Described herein, in certain embodiments, are recombinant adeno-associated viral (rAAV) vectors comprising, from 5′ to 3′: a) a first ITR sequence; b) a CAG promoter; c) a coding sequence for a human interferon; d) a WPRE sequence; e) a SV40 polyA sequence; and f) a second ITR sequence.

Described herein, in certain embodiments, are recombinant adeno-associated viral (rAAV) vectors comprising, from 5′ to 3′: a) a first ITR sequence; b) a CAG promoter; c) a minigene as set forth in SEQ ID NO: 31 or 51; d) a coding sequence for a CpG-depleted human interferon; e) a WPRE sequence; f) a SV40 polyA sequence; and g) a second ITR sequence.

Described herein, in certain embodiments, are recombinant adeno-associated viral (rAAV) vectors comprising, from 5′ to 3′: a) a first ITR sequence; b) a CAG promoter; c) a minigene as set forth in SEQ ID NO: 31 or 51; d) a coding sequence for a CpG-depleted human interferon; e) a WPRE sequence; f) a bGH polyA sequence; and g) a second ITR sequence.

### BRIEF DESCRIPTION OF THE DRAWINGS

The disclosure is more completely understood with reference to the following drawings.

FIG. 1A depicts an exemplary workflow of the mechanism of the rAAVs described herein.

FIG. 1B depicts an exemplary inducible AAV expression system plasmid map depicting the various component elements used in the AAV expression systems throughout the specific embodiments.

FIG. 2 depicts an exemplary constitutive AAV expression system plasmid map depicting various component elements used in the AAV expression systems throughout the specific embodiments.

FIG. 3 depicts data from an in vitro validation of inducible GFP fluorescence.

FIG. 4 depicts data of in vitro kinetics of inducible GFP fluorescence by viral dose escalation.

FIGS. 5A-5AD depict exemplary plasmid vector maps for plasmids 1-30. FIG. 5A depicts an exemplary plasmid, Plasmid #1, which includes a CAG promoter sequence, a human interferon beta (hIFNβ) gene sequence which has been CpG depleted, a polyadenylation signal sequence derived from SV40 (SV40 pA), and an ampicillin resistance (AmpR) gene. FIG. 5B depicts an exemplary plasmid, Plasmid #2, which includes a CAG promoter sequence, a minigene (Xon), a hIFNβ gene sequence which has been CpG depleted and which lacks an ATG codon, an SV40 pA, and an AmpR gene. FIG. 5C depicts an exemplary plasmid, Plasmid #3, which includes a CAG promoter sequence, an Xon, an hIFNβ gene sequence which has been CpG depleted and which lacks an ATG codon, a bovine growth hormone polyadenylation signal (bGH pA), and a kanamycin resistance (KanR) gene. FIG. 5D depicts an exemplary plasmid, Plasmid #4, which includes a CAG promoter sequence, an hIFNβ gene sequence which has been CpG depleted, a bGH pA, and a KanR gene. FIG. 5E depicts an exemplary plasmid, Plasmid #5, which includes a CAG promoter sequence, an Xon, an hIFNβ gene sequence which has been CpG depleted and which lacks an ATG codon, a bGH pA, and a KanR gene. FIG. 5F depicts an exemplary plasmid, Plasmid #6, which includes a CAG promoter sequence, an hIFNβ gene sequence which has been CpG depleted, a bGH pA, and a KanR gene. FIG. 5G depicts an exemplary plasmid, Plasmid #7, which includes a CAG promoter sequence, a wild-type hIFNβ gene sequence, a SV40 pA, and an AmpR gene. FIG. 5H depicts an exemplary plasmid, Plasmid #8, which includes a CAG promoter sequence, a hIFNβ gene sequence which has been CpG depleted, a bGH pA, and a KanR gene. FIG. 5I depicts an exemplary plasmid, Plasmid #9, which includes a CAG promoter sequence, an Xon, a hIFNβ gene sequence which has been CpG depleted and which lacks an ATG codon, a bGH pA, and a KanR gene. FIG. 5J depicts an exemplary plasmid, Plasmid #10, which includes a CAG promoter sequence, a hIFNβ gene sequence which has been CpG depleted, a bGH pA, and a KanR gene. FIG. 5K depicts an exemplary plasmid, Plasmid #11, which includes a CAG promoter sequence, an Xon, a hIFNβ gene sequence which has been CpG depleted and which lacks an ATG codon, a bGH pA, and a KanR gene. FIG. 5L depicts an exemplary plasmid, Plasmid #12, which includes a CAG promoter sequence, a hIFNβ gene sequence which has been CpG depleted, a bGH pA, and a KanR gene which has been CpG depleted. FIG. 5M depicts an exemplary plasmid, Plasmid #13, which includes a CAG promoter sequence, an Xon, a hIFNβ gene sequence which has been CpG depleted and which lacks an ATG codon, a bGH pA, and a KanR gene which has been CpG depleted. FIG. 5N depicts an exemplary plasmid, Plasmid #14, which includes a CAG promoter sequence, a hIFNβ gene sequence which has been CpG depleted, a bGH pA, and a KanR gene which has been CpG depleted. FIG. 5O depicts an exemplary plasmid, Plasmid #15, which includes a CAG promoter sequence, an Xon, a hIFNβ gene sequence which has been CpG depleted and which lacks an ATG codon, a bGH pA, and a KanR gene which has been CpG depleted. FIG. 5P depicts an exemplary plasmid, Plasmid #16, which includes a CAG promoter sequence, an enhanced green florescent protein (eGFP) gene sequence, a bGH pA, and a KanR gene. FIG. 5Q depicts an exemplary plasmid, Plasmid #17, which includes a CAG promoter sequence, an Xon, an eGFP gene sequence which lacks an ATG codon, a bGH pA, and a KanR gene. FIG. 5R depicts an exemplary plasmid, Plasmid #18, which includes a CAG promoter sequence, an eGFP gene sequence, a bGH pA, and a KanR gene which has been CpG depleted. FIG. 5S depicts an exemplary plasmid, Plasmid #19, which includes a CAG promoter sequence, an Xon, an eGFP gene sequence which lacks an ATG codon, a bGH pA, and a KanR gene which has been CpG depleted. FIG. 5T depicts an exemplary plasmid, Plasmid #20, which includes a CAG promoter sequence, an mCardinal gene sequence, a bGH pA, and a KanR gene which has been CpG depleted. FIG. 5U depicts an exemplary plasmid, Plasmid #21, which includes a CAG promoter sequence, an mCardinal gene sequence, a bGH pA, and a KanR gene which has been CpG depleted. FIG. 5V depicts an exemplary plasmid, Plasmid #22, which includes a CAG promoter sequence, an Xon, an mCardinal gene sequence which lacks an ATG codon, a bGH pA, and a KanR gene which has been CpG depleted. FIG. 5W depicts an exemplary plasmid, Plasmid #23, which includes a CAG promoter sequence, an Xon, an mCardinal gene sequence which lacks an ATG codon, a bGH pA, and a KanR gene which has been CpG depleted. FIG. 5X depicts an exemplary plasmid, Plasmid #24, which includes a CAG promoter sequence, a hIFNβ gene sequence which has been CpG depleted, a bGH pA, and a KanR gene which has been CpG depleted. FIG. 5Y depicts an exemplary plasmid, Plasmid #25, which includes a CAG promoter sequence, an mCardinal gene sequence, a bGH pA, and a KanR gene which has been CpG depleted. FIG. 5Z depicts an exemplary plasmid, Plasmid #26, which includes a CAG promoter sequence, a mouse interferon beta (mIFNβ) gene sequence, a bGH pA, and a KanR gene which has been CpG depleted. FIG. 5AA depicts an exemplary plasmid, Plasmid #27, which includes a CAG promoter sequence, a rat interferon beta (rIFNβ) gene sequence, a bGH pA, and a KanR gene which has been CpG depleted. FIG. 5AB depicts an exemplary plasmid, Plasmid #28, which includes a CAG promoter sequence, a canine interferon beta (cIFNβ) gene sequence, a bGH pA, and a KanR gene which has been CpG depleted. FIG. 5AC depicts an exemplary plasmid, Plasmid #29, which includes a CAG promoter sequence, an Xon, a hIFNβ gene sequence which has been CpG depleted and lacks an ATG codon, a bGH pA, and a KanR gene which has been CpG depleted. FIG. 5AD depicts an exemplary plasmid, Plasmid #30, which includes a CAG promoter sequence, an Xon, a hIFNβ gene sequence which has been CpG depleted and lacks an ATG codon, a bGH pA, and a KanR gene which has been CpG depleted.

FIG. 6 depicts an exemplary workflow for producing rAAV vector plasmids to be used to produce rAAV vectors as disclosed herein.

FIG. 7 depicts a sodium dodecyl sulfate polyacrylamide gel (SDS-PAGE) with Coomassie Blue assessing the purity of exemplary AAV preparations when 1e11 vg of the respective AAV preparation was loaded into a well on a 10% protein gel and submitted to electrophoresis for protein separation.

FIGS. 8A-8C depict data from an in vitro assessment of payload production efficiency. FIGS. 8A-8B depict the relative production efficacy of various AAV preparations expressed as a ratio of each vg titer (vg/mL) over the vg titer for the reference stock (AAV produced using Plasmid #1 (FIG. 8A) or Plasmid #12 (FIG. 8B)). Plasmids used to produce the corresponding AAV are identified on the X-axis. FIG. 8C depicts the vg titer obtained from 5 individual 1 liter (L) shake flasks of suspension cells using Plasmid #1.

FIGS. 9A-9E depict data from an in vitro assessment of payload expression. FIG. 9A depicts the payload expression measured for AAV preparations #6, #5, #8, and #9, each produced at a different vendor with different processes. All preparations were produced with Plasmid #1, carrying a human INFβ (hIFNβ) payload and packaged within the same AAV capsid serotype. FIG. 9B depicts the payload expression measured for AAV preparations #1, #9, #10, and #11 of various AAV genomes (produced from Plasmids #7, #1, #12 and #14, respectively) produced at one vendor using the same process and packaged within the same AAV capsid serotype. FIG. 9C depicts the payload expression measured for AAV preparations 6, #9, #10, #2, #13, and #16 with different AAV genomes (produced from Plasmids #1, #1, #12, #7, #24, and #25, respectively), expressing hIFNβ (except for AAV preparation #16, which contained a reporter payload (mCardinal) and was used as a negative control). Except for AAV preparation #6, all other preparations were performed at the same vendor using same process. FIG. 9D depicts the payload expression for AAV preparations #3, #6, #14, and #15 (produced from Plasmids #7, #1, #24, and #24, respectively). AAV preparations #14 and #15 both utilized Plasmid #24 but were produced at 2 different vendors. FIG. 9E depicts the payload expression measured for AAV preparations #6 and #17 (produced using Plasmids #1 and #26 and expressing human and mouse IFNβ, respectively) packaged in the same AAV serotype. For all plots, data represent the mean±SD of technical duplicates. Abbreviations: MOI, multiplicity of infection; prep, preparation.

FIG. 10 depicts the in vitro measurement of cytokine payload activity measured for six exemplary AAVs expressing hIFNβ (AAV preparations #6, #9, #10, #2, and #13), one AAV expressing a reporter payload (mCardinal; AAV preparation #12) and culture medium (NC) used as negative controls. Values represent the mean±SD for 8 technical duplicates.

FIG. 11 depicts the in vitro assessment of reduction in cancer cell viability upon exposure to an AAV expression system described herein expressing human IFNβ (AAV-hIFNβ). As depicted, the relative cell viability is expressed in percentage, at day 6 post-exposure to a low, medium, or high dose of AAV-hIFNβ or AAV-GFP. Staurosporin (STS) was used as a positive killing control, and the data is expressed as the mean±SD, with n=4 technical replicates.

FIG. 12 depicts the secreted hIFNβ levels from transduced GBM cells in vitro as measured with ELISA. Values represent the mean±SD of 4 technical replicates.

FIG. 13 depicts an exemplary process and assay for manufacturing of AAV constructs described herein.

### DETAILED DESCRIPTION

Cancer continues to be the second most common cause of death in the US after heart disease. In the US in 2023, a total of 1.9 million new cancer cases (about 5,370 each day) and 609,820 deaths from cancer are expected to occur (about 1,670 deaths per day). The therapeutic repertoire for many cancers (e.g., glioblastoma, metastatic brain tumors, and uveal melanoma) is still largely restricted to invasive and/or cytotoxic approaches, including, resection, radiation, chemotherapy, and combinations thereof. Even common immune-based oncologic drugs, such as cytokine therapies, exhibit negative characteristics including toxicity due to systemic administration, short half-lives, and lack of specificity. Further, methods for adequately delivering a therapeutic to a cancerous tissue are still problematic.

Improved therapies for treating cancers (e.g. glioblastoma, metastatic brain tumor, uveal melanoma) are needed, including cytokine therapies with improved characteristics, such as lower toxicity, long-term, steady, and durable expression, improved delivery (e.g., local delivery to the tumor), and low doses. Provided herein are compositions and methods relating to rAAV delivery of a payload of interest, e.g., a cytokine such as an interferon.

### Definitions

Unless defined otherwise, all technical and scientific terms used herein have the same meaning as is commonly understood by one of skill in the art to which the claimed subject matter belongs. Generally, nomenclatures utilized in connection with, and techniques of, immunology, oncology, cell and tissue culture, molecular biology, and protein and oligonucleotide or polynucleotide chemistry and hybridization described herein are those well-known and commonly used in the art. Units of measure not otherwise defined accord with The International System of Units (SI), NIST Special Publication 330, 2019 edition.

As used herein, all numerical values or numerical ranges comprise whole integers within or encompassing such ranges and fractions of the values or the integers within or encompassing ranges unless the context clearly indicates otherwise. Thus, for example, reference to a range of 90-100%, comprises 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold comprises 1-, 2-, 3-, 4-, 5-, 6-, 7-, 8-, 9-, 10-, 11-, 12-, 13-, 14-, 15-, 16-, 17-, 18-, 19-, or 20-fold, etc., as well as 1.1-, 1.2-, 1.3-, 1.4-, or 1.5-fold, etc., 2.1-, 2.2-, 2.3-, 2.4-, or 2.5-fold, etc., and so forth.

The terminology used herein is for the purpose of describing particular embodiments only and is not intended to be limiting of any embodiment. As used herein, the singular forms “a,” “an,” and “the” are intended to comprise the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” comprises any and all combinations of one or more of the associated listed items.

As used herein, the term “adeno-associated virus vector” or “AAV vector” refers to a vector derived from an adeno-associated virus serotype, comprising without limitation, AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16. AAV vectors can have one or more of the AAV wild-type genes deleted in whole or part, e.g., the Rep and/or Cap genes, but retain functional flanking inverted terminal repeat (ITR) sequences. Functional ITR sequences promote the rescue, replication, and packaging of the AAV virion. Thus, an AAV vector is defined herein to comprise at least those sequences required in cis for replication and packaging (e.g., functional ITRs) of the virus. ITRs do not need to be the wild-type polynucleotide sequences and, in some embodiments, are altered, e.g., by the insertion, deletion, or substitution of nucleotides, so long as the sequences provide for functional rescue, replication, and packaging.

The terms “adeno-associated virus inverted terminal repeats” or “AAV ITRs” refer to regions flanking each end of the AAV genome which function together in cis as origins of DNA replication and as packaging signals for the virus. As used herein, an “AAV ITR” does not necessarily comprise the wild-type polynucleotide sequence, which, in some embodiments, are altered, e.g., by the insertion, deletion or substitution of nucleotides. Additionally, the AAV ITRs are derived from any of several AAV serotypes, comprising without limitation AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP.B, AAV-PHP.EB, AAV-2.5, AAV2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, among others. Furthermore, 5′ and 3′ ITRs which flank a selected polynucleotide sequence in an AAV vector need not be identical or derived from the same AAV serotype or isolate, so long as they function as intended, e.g., to allow for the desired therapeutic or genome editing effect. Additionally, AAV ITR modifications to the D-element can code for or facilitate different configurations of AAV genomes, single-stranded AAV genomes (i.e., ssAAV), or self-complementary AAV genomes (i.e., scAAV).

Unless specifically stated or obvious from context, as used herein, the term “about” in reference to a number or range of numbers is understood to mean the stated number and numbers ±10% thereof, or 10% below the lower listed limit and 10% above the higher listed limit for the values listed for a range.

As used herein, “CpG dinucleotide,” “CpG sites,” or “CpG” refers to regions of a nucleic acid (e.g., DNA or RNA) where a cytosine nucleotide occurs next to a guanine nucleotide in a linear nucleic acid sequence of nucleotides along its length, e.g., —C— phosphate-G-, cytosine and guanine separated by only one phosphate, or cytosine 5′ to the guanine nucleotide.

The terms “recipient”, “individual”, “subject”, “host”, and “patient”, are used interchangeably herein and in some embodiments, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys, etc. In some embodiments, the mammal is human. None of these terms require the supervision of medical personnel.

“Percent identity,” “% identity,” or “sequence identity” refer to the extent to which two sequences (nucleotide or amino acid) have the same residues at the same positions in an alignment. For example, “a nucleotide sequence is X % identical to SEQ ID NO: Y” refers to % identity of the nucleotide sequence to SEQ ID NO: Y and is elaborated as X % of residues in the nucleotide sequence are identical to the corresponding residues of sequence disclosed in SEQ ID NO: Y. A sequence said to be X % identical to a reference sequence may contain more nucleotide or amino acid residues than specified in the reference sequence but must contain a sequence corresponding to the reference sequence. In most cases, the sequence in question will contain a sequence that corresponds to all of the specified reference sequences. Generally, computer programs are employed for such calculations. Exemplary programs that compare and align pairs of sequences, comprise ALIGN, FASTA, gapped BLAST, BLASTP, BLASTN, or GCG.

“Polynucleotide,” or “nucleic acid,” are used interchangeably herein and refer to chains of nucleotides of any length, and comprise DNA or RNA. In some embodiments, the nucleotides are deoxyribonucleotides, ribonucleotides, modified nucleotides or bases, and/or their analogs. A polynucleotide may comprise modified nucleotides, such as methylated nucleotides and their analogs (e.g., increased CpG dinucleotides, as described). If present, modification to the nucleotide structure is imparted before or after assembly of the chain. In some embodiments, the sequence of nucleotides is interrupted by non-nucleotide components. In some embodiments, a polynucleotide is further modified after polymerization, such as by conjugation with a labeling component. Other types of modifications comprise, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates) and with charged linkages (e.g., phosphorothioates, phosphorodithioates), those containing pendant moieties, such as, for example, proteins (e.g., nucleases, toxins, antibodies, signal peptides, poly-L-lysine), those with intercalators (e.g., acridine, psoralen), those containing chelators (e.g., metals, radioactive metals, boron, oxidative metals), those containing alkylators, those with modified linkages (e.g., alpha anomeric nucleic acids), as well as unmodified forms of the polynucleotide(s). In some embodiments, any of the hydroxyl groups ordinarily present in the sugars are replaced, for example, by phosphonate groups, phosphate groups, protected by standard protecting groups, or activated to prepare additional linkages to additional nucleotides, or are conjugated to solid supports. In some embodiments, 5′ and 3′ terminal OH is phosphorylated or substituted with amines or organic capping group moieties of from 1 to 20 carbon atoms. Other hydroxyls may also be derivatized to standard protecting groups. In some embodiments, polynucleotides also contain analogous forms of ribose or deoxyribose sugars, comprising, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. In some embodiments, one or more phosphodiester linkages are replaced by alternative linking groups. These alternative linking groups comprise, but are not limited to, embodiments wherein phosphate is replaced by P(O)S (“thioate”), P(S)S (“dithioate”), (O)NRi (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, comprising RNA and DNA. In some embodiments, nucleic acids or polynucleotides disclosed herein have reduced CpG dinucleotides as compared to a parental equivalent. In some embodiments, nucleic acids or polynucleotides disclosed herein have depleted CpG dinucleotides (i.e., all CpG dinucleotides have been modified to no longer be CpG dinucleotides or all CpG dinucleotides have been deleted) as compared to a parental equivalent. In some embodiments, nucleic acids or polynucleotides disclosed herein have increased methylated CpG dinucleotides.

The phrase “pharmaceutically acceptable” refers to molecular entities and compositions that are physiologically tolerable to a mammal, particularly a human or animal patient.

A “recombinant AAV vector (rAAV vector)” refers to a polynucleotide vector derived from an AAV and comprising one or more heterologous sequences (i.e., nucleic acid sequence not of AAV origin) flanked by at least one AAV ITR. In some embodiments, such rAAV vectors are replicated and packaged into viral particles when introduced into a host cell that has a suitable helper polynucleotide or virus (or that is expressing suitable helper functions) and that expresses AAV Rep and Cap gene products (i.e. AAV Rep and Cap proteins). When a rAAV vector is incorporated into a larger polynucleotide (e.g., in a chromosome or in another vector such as a plasmid used for cloning or transfection), then the rAAV vector is referred to as a “pro-vector” which is “rescued” by replication and encapsidation in the presence of AAV packaging functions and suitable helper functions.

As used herein, the term “treatment” (also “treat” or “treating”) refers to any administration of a substance or composition (e.g., an AAV vector as described herein) that partially or completely alleviates, ameliorates, relives, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein, “operably linked”, “operable linkage”, “operatively linked”, or grammatical equivalents thereof refer to an arrangement of genetic elements, e.g., a promoter, an enhancer, a polyadenylation sequence, etc., wherein an operation (e.g., movement or activation) of a first genetic element has some effect on the second genetic element. The effect on the second genetic element can be, but need not be, of the same type of operation of the first genetic element. For example, two genetic elements are operably linked if movement of the first element causes an activation of the second element. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

The terms “peptide,” “polypeptide,” and “protein” as used herein mean a chain of amino acids. The term protein, as used herein further means a large molecule comprising one or more chains of amino acids and, in some embodiments, is a fragment or domain of a protein or a full length protein. Furthermore, as used herein, the term protein either refers to a linear chain of amino acids or to a chain of amino acids that has been processed and folded into a functional protein. The protein structure is divided into four distinct levels: (1) primary structure—referring to the sequence of amino acids in the polypeptide chain, (2) secondary structure—referring to the regular local sub-structures on the polypeptide backbone chain, such as α-helix and β-sheets, (3) tertiary structure—referring to the three-dimensional structure if monomeric and multimeric protein molecules, and (4) quaternary structure—referring to the three-dimensional structure comprising the aggregation of two or more individual polypeptide chains that operate as a single functional unit. The use of peptide or polypeptide herein does not mean that the chain of amino acids is not also a protein (i.e., a chain of amino acids having a secondary, tertiary or quaternary structure).

As used herein the term “wild type” or “wild-type” refers to the form of an organism, strain, gene, nucleic acid, vector, or vector components as it occurs naturally in nature as distinguished from mutant or variant forms.

Recombinant Adeno-Associated Viral (rAAV) Vectors of the Disclosure

Cytokines are regulators of innate and adaptive immunity that enable cells of the immune system to communicate. Because of the ability of the immune system to recognize and destroy cancer cells, there has been considerable interest in harnessing cytokines for the treatment of cancer. However, current cytokine therapies exhibit negative characteristics including toxicity due to systemic administration, short half-lives, and lack of specificity. Improved cytokine therapies for treating cancers (e.g. glioblastoma, metastatic brain tumor, uveal melanoma) are needed.

Provided herein are compositions and methods relating to rAAV delivery of a cytokine payload of interest that allow for targeted, low-dose, direct delivery to the tumor and the tumor microenvironment. Local tumor delivery and low dosing of rAAVs can result in a reduction of local and systemic toxicities, for example, by bypassing neutralizing antibodies that occur during systemic administration of rAAVs. Further, rAAVs described herein have longer half-lives enabling long-term, durable, and stable expression and are also self-limiting as payload activity generally ceases with tumor death. Compositions and methods described herein can further provide for reduced non-specific inflammation by being optimized immunologically and for vector integrity.

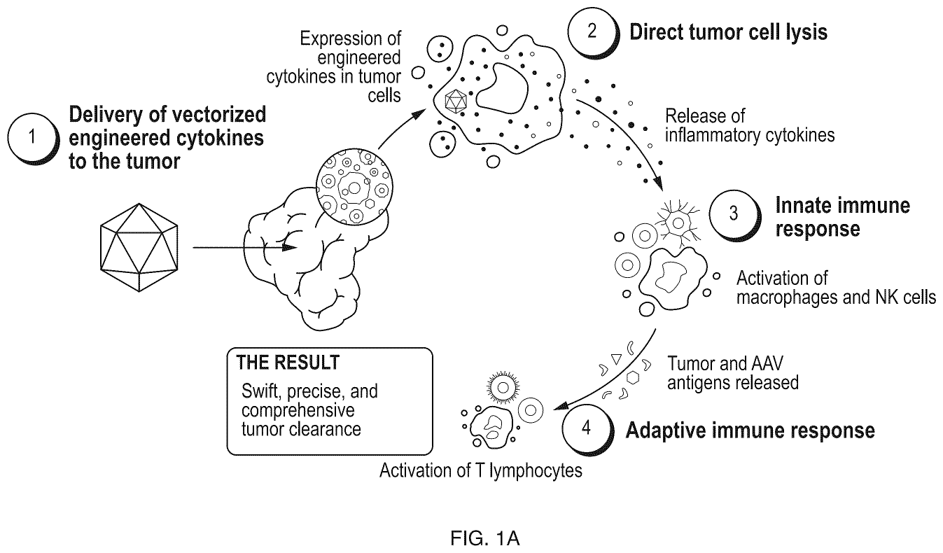

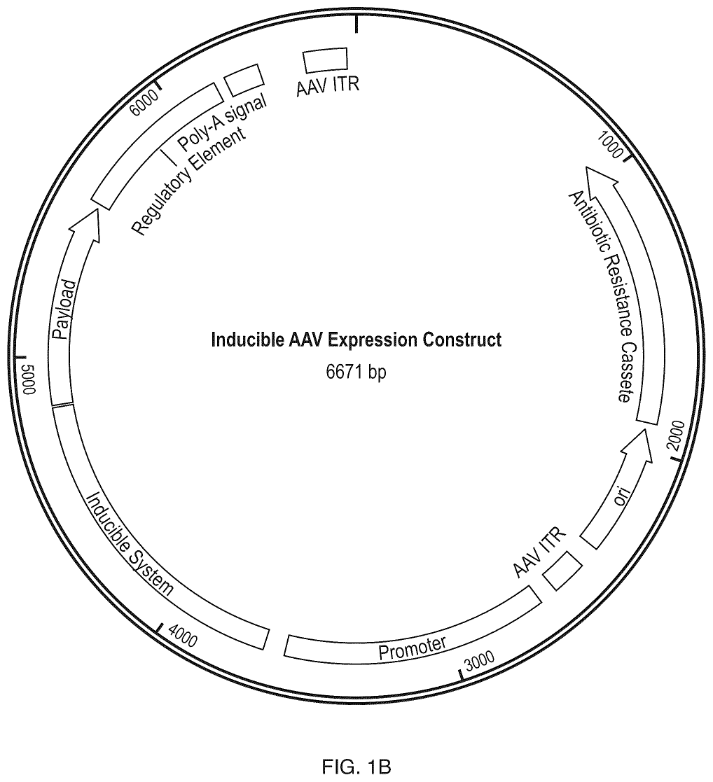

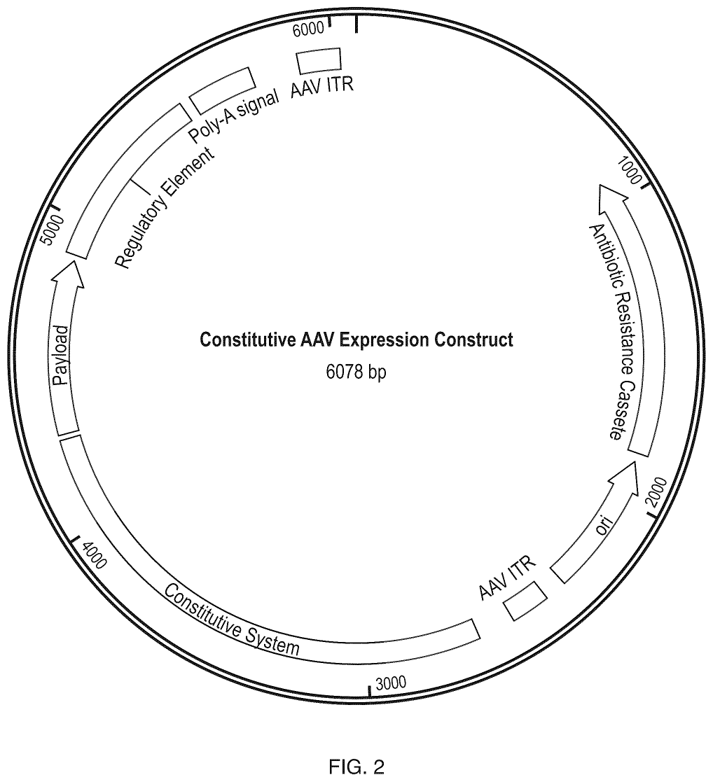

An exemplary workflow of the mechanism of the rAAVs described herein is seen in FIG. 1A. rAAVs described herein allow for local delivery to the tumor (1), which in turn results in expression of engineered cytokines in the tumor cells and direct tumor cell lysis. As a result, inflammatory cytokines are released triggering an innate immune response (3) and activation of macrophages and natural killer cells to clear the tumor and AAV antigens released followed by an adaptive immune response (4). As a result, the tumor is cleared quickly with reduced local and systemic toxicities.

Described herein, in certain embodiments, are recombinant adeno-associated viral (rAAV) vectors comprising, from 5′ to 3′: a) a promoter; b) a polynucleotide encoding a payload of interest; and c) a polynucleotide comprising a regulatory element. In some embodiments, the regulatory element is an inducible regulatory element (e.g., an inducible system, such as a splice modulator system and/or promoter). In some embodiments, the regulatory element is a constitutively active regulatory element.

Further described herein, in certain embodiments, are recombinant adeno-associated viral (rAAV) vectors comprising, from 5′ to 3′: a) a first ITR sequence; b) a CAG promoter; c) a coding sequence for an interferon; d) a WPRE sequence; e) a SV40 polyA sequence; and f) a second ITR sequence.

Further described herein, in certain embodiments, are recombinant adeno-associated viral (rAAV) vectors comprising, from 5′ to 3′: a) a promoter; and b) a polynucleotide encoding a payload of interest, the polynucleotide comprising reduced CpG dinucleotides and/or increased methylation of CpG dinucleotides as compared to a parental equivalent.

Further described herein, in certain embodiments, are recombinant adeno-associated viral (rAAV) vectors comprising, from 5′ to 3′: a) a first ITR sequence; b) a CAG promoter; c) a coding sequence for a CpG-depleted interferon; d) a WPRE sequence; e) a SV40 polyA sequence; and f) a second ITR sequence.

Further described herein, in certain embodiments, are recombinant adeno-associated viral (rAAV) vectors comprising, from 5′ to 3′: a) a first ITR sequence; b) a CAG promoter; c) a coding sequence for a CpG-depleted interferon; d) a WPRE sequence; e) a bGH polyA sequence; and f) a second ITR sequence.

Further described herein, in certain embodiments, are recombinant adeno-associated viral (rAAV) vectors comprising, from 5′ to 3′: a) a promoter; b) a minigene comprising a splice modulator binding site; and c) a polynucleotide encoding a payload of interest, the polynucleotide comprising reduced CpG dinucleotides and/or increased methylation of CpG dinucleotides as compared to a parental equivalent.

Further described herein, in certain embodiments, are recombinant adeno-associated viral (rAAV) vectors comprising, from 5′ to 3′: a) a first ITR sequence; b) a CAG promoter; c) a minigene as set forth in SEQ ID NO: 31; d) a coding sequence for a CpG-depleted human interferon; e) a WPRE sequence; f) a SV40 polyA sequence; and g) a second ITR sequence.

Further described herein, in certain embodiments, are recombinant adeno-associated viral (rAAV) vectors comprising, from 5′ to 3′: a) a first ITR sequence; b) a CAG promoter; c) a minigene as set forth in SEQ ID NO: 51; d) a coding sequence for a CpG-depleted interferon; e) a WPRE sequence; f) a SV40 polyA sequence; and g) a second ITR sequence.

Further described herein, in certain embodiments, are rAAV vectors comprising, from 5′ to 3′: a) a promoter; b) a regulatory element comprising a splice modulator binding site; and c) a polynucleotide encoding a payload of interest, the polynucleotide comprising reduced CpG dinucleotides and/or increased methylation of CpG dinucleotides as compared to a parental equivalent.

Further described herein, in certain embodiments, are rAAV vectors comprising, from 5′ to 3′: a) a first ITR sequence; b) a CAG promoter; c) a regulatory element as set forth in SEQ ID NO: 31; d) a coding sequence for a CpG-depleted an interferon; e) a WPRE sequence; f) a bGH polyA sequence; and g) a second ITR sequence.

Further described herein, in certain embodiments, are rAAV vectors comprising, from 5′ to 3′: a) a first ITR sequence; b) a CAG promoter; c) a regulatory element as set forth in SEQ ID NO: 51; d) a coding sequence for a CpG-depleted an interferon; e) a WPRE sequence; f) a bGH polyA sequence; and g) a second ITR sequence.

### CpG Dinucleotide Modification

Described herein, in certain embodiments, are recombinant adeno-associated viral (rAAV) vectors comprising reduced CpG dinucleotides as compared to a parental equivalent. In some embodiments, the rAAV vectors comprise increased methylation of CpG dinucleotides as compared to a parental equivalent. In some embodiments, the rAAV vectors comprise depleted CpG dinucleotides as compared to a parental equivalent. In some embodiments, the rAAV vectors comprise reduced CpG dinucleotides and increased methylation of CpG dinucleotides. In some embodiments, a CpG dinucleotide is said to be depleted or reduced if one or more nucleotides of said CpG dinucleotide are substituted with one or more different nucleotides so that it is no longer a CpG dinucleotide sequence. In some embodiments, if a CpG dinucleotide of a rAAV comprising a coding sequence (e.g., for a regulatory element, gene (e.g., transgene), antibiotic resistance gene, etc.) is substituted with one or more different nucleotides, the one or more different nucleotides are selected such that the function of the coding sequence is retained. In other words, if said coding sequence is a regulatory element such as a promoter or enhancer, the promoter or enhancer function is retained, or if said coding sequence codes for a polypeptide, the polypeptide amino acid sequence is retained and/or the polypeptide function is retained.

In some embodiments, the rAAV vectors comprise reduced CpG dinucleotides as compared to a parental equivalent. In some embodiments, CpG dinucleotides are reduced by at least about 5%, 10%, 15%, 20%, 25%, 30%, 35%, 40%, 45%, 50%, 55%, 60%, 65%, 70%, 75%, 80%, 85%, 90%, 95%, or more than 99% as compared to a parental equivalent. In some embodiments, CpG dinucleotides are reduced in a range of about 5% to about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 10% to about 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 15% to about 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 20% to about 25%, 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 25% to about 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 30% to about 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 40% to about 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 50% to about 55%, 60%, 65%, 70%, 75%, 80%, 85%, 90%, 95%, 96%, 97%, 98%, 99%, or 100%. In some embodiments, CpG dinucleotides are reduced in a range of about 60% to about 65%, 70%, 75%, 80%, 85%, 90%, 95%, 96%, 97%, 98%, 99%, or 100%. In some embodiments, CpG dinucleotides are reduced in a range of about 70% to about 75%, 80%, 85%, 90%, 95%, 96%, 97%, 98%, 99%, or 100%. In some embodiments, CpG dinucleotides are reduced in a range of about 80% to about 85%, 90%, 95%, 96%, 97%, 98%, 99%, or 100%. In some embodiments, CpG dinucleotides are reduced in a range of about 90% to about 95%, 96%, 97%, 98%, 99%, or 100%. In some embodiments, CpG dinucleotides are reduced by at least about 50%. In some embodiments, CpG dinucleotides are reduced by at least about 75%.

In some embodiments, the rAAV vectors comprise depleted CpG dinucleotides as compared to a parental equivalent. In some embodiments, CpG dinucleotides are reduced by deletion of certain CpG dinucleotides. In some embodiments, CpG dinucleotides are reduced by substitution of one or more nucleotides within a CpG dinucleotide to create a sequence that is not a CpG dinucleotide. In some embodiments, CpG dinucleotides are depleted by deletion of all CpG dinucleotides. In some embodiments, CpG dinucleotides are depleted by substitution of one or more nucleotides within each CpG dinucleotide to create sequences that are not CpG dinucleotides.

In some embodiments, the rAAV vectors comprise increased methylation of CpG dinucleotides as compared to a parental equivalent. In some embodiments, methylation of CpG dinucleotides is increased by at least about 5%, 10%, 15%, 20%, 25%, 30%, 35%, 40%, 45%, 50%, 55%, 60%, 65%, 70%, 75%, 80%, 85%, 90%, 95%, or more than 95% as compared to a parental equivalent. In some embodiments, methylation of CpG dinucleotides is increased in a range of about 5% to about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 10% to about 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 15% to about 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 20% to about 25%, 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 25% to about 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 30% to about 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 40% to about 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 50% to about 55%, 60%, 65%, 70%, 75%, 80%, 85%, 90%, 95%, 96%, 97%, 98%, 99%, or 100%. In some embodiments, methylation of CpG dinucleotides is increased in a range of about 60% to about 65%, 70%, 75%, 80%, 85%, 90%, 95%, 96%, 97%, 98%, 99%, or 100%. In some embodiments, methylation of CpG dinucleotides is increased in a range of about 70% to about 75%, 80%, 85%, 90%, 95%, 96%, 97%, 98%, 99%, or 100%. In some embodiments, methylation of CpG dinucleotides is increased in a range of about 80% to about 85%, 90%, 95%, 96%, 97%, 98%, 99%, or 100%. In some embodiments, methylation of CpG dinucleotides is increased in a range of about 90% to about 95%, 96%, 97%, 98%, 99%, or 100%.

In some embodiments, CpG dinucleotides are modified (e.g., reduced, depleted, and/or methylation is increased) across the entire length of a provided rAAV vector. In some embodiments, CpG dinucleotides are modified only in one or more critical portions, or elements, of a provided rAAV vector (e.g., in a regulatory element, a polynucleotide comprising a coding sequence, etc.). The present disclosure provides, among other things, the insight that certain polynucleotides encoding payloads of interest (e.g., polynucleotides encoding one or more interferons, such as IFNβ) exhibit superior therapeutic results (e.g., increased efficacy, lower toxicity, etc.) when CpG dinucleotides are modified within the coding sequence for said payload of interest. Thus, in one non-limiting example, a rAAV vector provided by the present disclosure comprises a polynucleotide encoding an interferon, where the coding sequence of said interferon comprises modified CpG dinucleotides (e.g., reduced, depleted, and/or methylation is increased) as compared to a parental equivalent, but the other elements in the flanking nucleic acid do not have modified CpG dinucleotides. In another non-limiting example, a rAAV vector provided by the present disclosure comprises a polynucleotide encoding an interferon, where the coding sequence of said interferon comprises modified CpG dinucleotides (e.g., reduced, depleted, and/or methylation is increased) as compared to a parental equivalent, and one or more other polynucleotides in said rAAV vector comprises modified CpG dinucleotides (e.g., a promoter, an antibiotic resistance gene, a minigene, and/or any polynucleotide element described herein).

### Minigene Regulation

In some embodiments, a regulatory element of the present disclosure is an inducible regulatory element (e.g., an inducible system, such as a splice modulator system and/or promoter). In some embodiments, an inducible regulatory element is a minigene. In some embodiments, a regulatory element is a constitutive regulatory element.

Described herein, in some embodiments, are recombinant adeno-associated viral (rAAV) vectors comprising a minigene that comprises a splice modulator binding site. In some embodiments, a minigene is located 5′ to a polynucleotide encoding a payload of interest. In some embodiments, the rAAVs described herein comprise one or more (e.g., 1, 2, 3, 4, 5, 6, 7, 8) exons, introns, pseudoexons, fragments thereof, or combinations thereof.

In some embodiments, a minigene comprises a minimal gene fragment that includes at least one exon and a control region, or splice modulator site, necessary for the minimal gene fragment to regulate expression of downstream polynucleotide sequences. In some embodiments, a minigene comprises one or more (e.g., 1, 2, 3, 4, 5, 6, 7, 8) exons, introns, pseudoexons, fragments thereof, or combinations thereof. In many embodiments, a minigene splice modulator site controls the splicing outcome of an RNA transcript encoded by the minigene. In some embodiments, the splice modulator site guides an RNA transcript toward a certain splicing outcome based on factors present in a certain cell or tissue context (e.g., splicing outcome 1 occurs if the minigene is present in a liver cell, splicing outcome 2 occurs if the minigene is present in a retinal cell, splicing outcome 3 occurs if the minigene is present in a kidney cell, etc.). In some embodiments, the splice modulator site guides an RNA transcript toward a certain splicing outcome due to binding of said splice modulator site, e.g., at one or more nucleic acid sequences, with a splice modulator. In some embodiments, the splice modulator is a polypeptide, nucleic acid, or small molecule. In some embodiments, a minigene is used to regulate expression of a downstream polynucleotide sequence by engineering said minigene to only allow (or activate) expression of said downstream polynucleotide sequence in the presence of a splice modulator and/or in the context of a specific cell/tissue type.

In some embodiments, a minigene encodes an in-frame translation stop codon. In some embodiments, alternative splicing of a minigene transcript removes the in-frame translation stop codon. In some embodiments, alternative splicing of a minigene transcript modifies the transcript thereby deleting or nullifying the stop codon, introducing an initiation or start codon, restoring the open reading frame, or providing a missing portion of the protein. In some embodiments, alternative splicing of a minigene transcript allows for transcription and translation of a downstream polynucleotides, e.g., a polynucleotide encoding a payload of interest, as described herein.

In some embodiments, a polynucleotide downstream from a minigene (e.g., a polynucleotide encoding a payload of interest, as described herein) comprises a translation stop codon. In some embodiments, a polynucleotide downstream from a minigene does not comprise a start codon. In some embodiments, a polynucleotide downstream from a minigene does not comprise an open reading frame.

In some embodiments, a minigene and a polynucleotide encoding a payload of interest are linked by a cleavable peptide. In some embodiments, the cleavable peptide is a self-cleaving peptide, a drug-sensitive protease, or a substrate for an endogenous endoprotease.

In some embodiments, the minigene comprises the nucleic acid sequence of SEQ ID NO: 31. In some embodiments, the minigene comprises a nucleic acid sequence having at least 70% (e.g., 75%, 80%, 90%, 95%, 97%, 98%, or 99%) sequence identity to the nucleic acid sequence of SEQ ID NO: 31. In some embodiments, the minigene comprises a nucleic acid sequence having at least 75% sequence identity to the nucleic acid sequence of SEQ ID NO: 31. In some embodiments, the minigene comprises a nucleic acid sequence having at least 80% sequence identity to the nucleic acid sequence of SEQ ID NO: 31. In some embodiments, the minigene comprises a nucleic acid sequence having at least 90% sequence identity to the nucleic acid sequence of SEQ ID NO: 31. In some embodiments, the minigene comprises a nucleic acid sequence having at least 95% sequence identity to the nucleic acid sequence of SEQ ID NO: 31. In some embodiments, the minigene comprises a nucleic acid sequence having at least 97% sequence identity to the nucleic acid sequence of SEQ ID NO: 31. In some embodiments, the minigene comprises a nucleic acid sequence having at least 98% sequence identity to the nucleic acid sequence of SEQ ID NO: 31. In some embodiments, the minigene comprises a nucleic acid sequence having at least 99% sequence identity to the nucleic acid sequence of SEQ ID NO: 31.

In some embodiments, the minigene comprises the nucleic acid sequence of SEQ ID NO: 51. In some embodiments, the minigene comprises a nucleic acid sequence having at least 70% (e.g., 75%, 80%, 90%, 95%, 97%, 98%, or 99%) sequence identity to the nucleic acid sequence of SEQ ID NO: 51. In some embodiments, the minigene comprises a nucleic acid sequence having at least 75% sequence identity to the nucleic acid sequence of SEQ ID NO: 51. In some embodiments, the minigene comprises a nucleic acid sequence having at least 80% sequence identity to the nucleic acid sequence of SEQ ID NO: 51. In some embodiments, the minigene comprises a nucleic acid sequence having at least 90% sequence identity to the nucleic acid sequence of SEQ ID NO: 51. In some embodiments, the minigene comprises a nucleic acid sequence having at least 95% sequence identity to the nucleic acid sequence of SEQ ID NO: 51. In some embodiments, the minigene comprises a nucleic acid sequence having at least 97% sequence identity to the nucleic acid sequence of SEQ ID NO: 51. In some embodiments, the minigene comprises a nucleic acid sequence having at least 98% sequence identity to the nucleic acid sequence of SEQ ID NO: 51. In some embodiments, the minigene comprises a nucleic acid sequence having at least 99% sequence identity to the nucleic acid sequence of SEQ ID NO: 51.

In some embodiments, the minigene is regulated by a small molecule splicing modifier. In some embodiments, the small molecule splicing modifier is sudemycin (FR901464, pladienolide B), LMI070, RG7916, or RG7800 and derivatives thereof.

In some embodiments, the small molecule splicing modifier is LMI070 (CAS No.: 1562338-42-4) having the following structure:

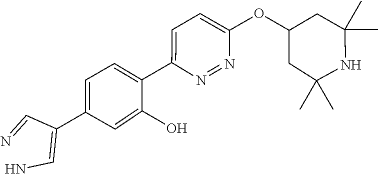 [SMILES: CC1(C)CC(Oc2ccc(-c3ccc(/C(C=N)=C/N)cc3O)nn2)CC(C)(C)N1]

In some embodiments, the small molecule splice modulator is RG7916 (Roche/PTC/SMAF, 7-(4,7-diazaspiro[2.5]octan-7-yl)-2-(2,8-dimethylimidazo[1,2-b]pyridazin-6-yl)-4H-pyrido[1,2-a]pyrimidin-4-one) (CAS No.: 1825352-65-5) having the following structure:

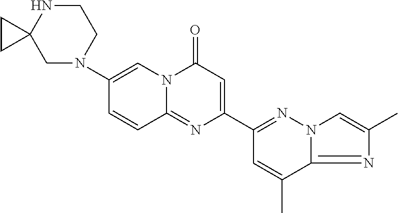 [SMILES: Cc1cn2nc(-c3cc(=O)n4cc(N5CCNC6(CC6)C5)ccc4n3)cc(C)c2n1]

In some embodiments, the small molecule splice modulator is RG7800 (Roche) (CAS No.: 1449598-06-4) having the following structure:

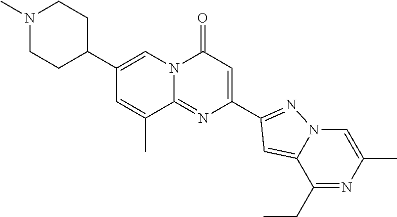

In some embodiments, the small molecule splice modulator is an analogue of RG7916 or RG7800.

In some embodiments, the small molecule splice modulator is a sudemycin selected from the group consisting of: (5′,Z)-5-(((1R,4R)-4-((2JE′,4JE)-5-((3R,55′)-7,7-dimethyl-1,6-dioxaspiro[2.5]octan-5-yl)-3-methylpenta-2,4-dien-1-yl)cyclohexyl)amino)-5-oxopent-3-en-2-yl methylcarbamate and (5′,Z)-5-(((1R,4R)-4-((2JE′,4JE)-5-((3R,55′)-7,7-dimethyl-1,6-dioxaspiro[2.5]octan-5-yl)-3-methylpenta-2,4-dien-1-yl)cyclohexyl)amino)-5-oxopent-3-en-2-yl dimethylcarbamate.

In some embodiments, the small molecule splice modulator is a pladienolide compound. One example of a pladienolide compound is (8E,12E,14E)-7-((4-Cycloheptylpiperazin-1-yl)carbonyl)oxy-3,6,16,21-tetrahydroxy-6,10,12,16,20-pentamethyl-18,19-epoxytricosa-8,12,14-trien-11-olide, also known as E7107, which is a semisynthetic derivative of the natural product pladienolide D.

In some embodiments, the minigene is regulated by a disease state in a cell. In some embodiments, the disease state is cancer. In some embodiments, the cancer is glioblastoma. In some embodiments, the minigene is regulated by a cell type or tissue type.

### Payloads of Interest

Disclosed herein, in certain embodiments, are polynucleotides encoding a payload of interest. In some embodiments, a payload of interest is a therapeutic agent, such as a therapeutic polypeptide.

In some embodiments, the therapeutic agent is any agent that elicits a desired pharmacological effect when administered to an organism. In some embodiments, an agent is considered to be a therapeutic agent if it demonstrates a statistically significant effect across an appropriate population. In some embodiments, the appropriate population is a population of model organisms. In some embodiments, an appropriate population is defined by various criteria, such as a certain age group, sex, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, the therapeutic agent is an agent that has been or is required to be approved by a government agency before it can be marketed for administration to humans. In some embodiments, the therapeutic agent is an agent for which a medical prescription is required for administration to humans. In some embodiments, the therapeutic agent is a therapeutic polypeptide or a therapeutic polynucleotide. In some embodiments, the therapeutic polypeptide is a cytokine (e.g., an interferon).

In some embodiments, the therapeutic polypeptide is a cytokine. In some embodiments, the cytokine is a colony stimulating factor (CSF), a transforming growth factor (e.g., transforming growth factor-beta), a tumor necrosis factor (e.g., tumor necrosis alpha), an interleukin, or an interferon. In some embodiments, the cytokine is an interferon.

In some embodiments, the interleukin-1 alpha (IL-1 alpha), interleukin-1 beta (IL-1 beta), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin-10 (IL-10), interleukin-11 (IL-11), interleukin-12 (IL-12), interleukin-13 (IL-13), interleukin-14 (IL-14), interleukin-15 (IL-15), interleukin-16 (IL-16), interleukin-17 (IL-17), interleukin-18 (IL-18), interleukin-19 (IL-19), interleukin-20 (IL-20), interleukin-21 (IL-21), interleukin-22 (IL-22), interleukin-23 (IL-23), interleukin-24 (IL-24), interleukin-25 (IL-25), interleukin-26 (IL-26), interleukin-27 (IL-27), interleukin-28 (IL-28), interleukin-29 (IL-29), interleukin-30 (IL-30), interleukin-31 (IL-31), interleukin-32 (IL-32), interleukin-33 (IL-33), interleukin-34 (IL-34), interleukin-35 (IL-35), or interleukin-36 (IL-36)

In some embodiments, the colony stimulating factor (CSF) is CSF1 (M-CSF), CSF2 (GM-CSF), or CSF3 (G-CSF).

In some embodiments, the transforming growth factor (TGF) is TGF-β1, TGF-β2, or TGF-β3.

In some embodiments, the tumor necrosis factor (TNF) is TNF-α, TNF-β, or LT-β.

In some embodiments, the interferon (IFN) is a Type I IFN, a Type II IFN, or a Type III IFN. In some embodiments, the IFN is IFN alpha (IFNα), IFN beta (IFNβ), IFN gamma (IFNγ), IFN epsilon (IFNε), IFN kappa (IFNκ), IFN omega (IFNω), IFN lambda (IFNλ), IFN chi (IFNχ), IFN xi (IFNξ), IFN tau (IFNτ), IFN delta (IFNδ), IFN nu (IFNν), IFN zeta (IFNζ), IFN alfa, and derivatives thereof. In some embodiments, the interferon is IFNα, IFNβ, IFNγ, or combinations thereof. In some embodiments, an interferon is IFNα, IFNβ, IFNγ, IFNε, IFNκ, IFNω, IFNλ, IFNγ, IFNχ, IFNξ, IFNδ, IFNν, IFNζ, IFN alfa, or variants or derivatives thereof. In some embodiments, the IFN is IFN alpha-1, IFN alpha-2, IFN alpha-4, IFN alpha-5, IFN alpha-6, IFN alpha-7, IFN alpha-8, IFN alpha-10, IFN alpha-13, IFN alpha-14, IFN alpha-16, IFN alpha-17, IFN alpha-21, or variants or derivatives thereof. In some embodiments, the IFN is IFN-β1, IFN-β2, IFN-β3 or variants or derivatives thereof.

In some embodiments, the IFN is mouse, rat, equine, ruminant (e.g., sheep, cow, goat), primate (e.g., chimpanzee, baboon, gorilla, orangutan, monkey), dog, cat, pig, donkey, rabbit, fish, fly, human, non-primate placental mammal, or non-rodent placental mammal. In some embodiments, the IFN is a human IFNβ. In some embodiments, the IFN is human IFNα, human IFNβ, human IFNγ, human IFNω, human IFNε, human IFNκ, human IFNτ, human IFNζ, human IFN alfa, or variants or derivatives thereof. In some embodiments, the IFN is mouse IFNα, mouse IFNβ, mouse IFNγ, or variants or derivatives thereof. In some embodiments, the IFN is a mouse IFNα, IFNβ, IFNγ, IFNε, IFNκ, IFNω, IFNλ, IFNζ, or variants or derivatives thereof. In some embodiments, the IFN is a mouse IFNβ. In some embodiments, the IFN is a canine IFNα, IFNβ, IFNγ, IFNε, IFNκ, IFNω, IFNλ, or variants or derivatives thereof. In some embodiments, the IFN is a canine IFNβ, or variants or derivatives thereof. In some embodiments, the IFN is a rat IFNα, IFNβ, IFNγ, IFNε, IFNκ, IFNω, IFNλ, or variants or derivatives thereof. In some embodiments, the IFN is a rat IFNβ, or variants or derivatives thereof. In some embodiments, the IFN is a guinea pig IFNα, IFNβ, IFNγ, IFNε, IFNκ, IFNω, IFNλ, or variants or derivatives thereof. In some embodiments, the IFN is a guinea pig IFNβ, or variants or derivatives thereof. In some embodiments, the IFN is a non-primate and non-rodent placental mammal IFNδ, or variants or derivatives thereof. In some embodiments, the IFN is a placental mammal IFNε, IFNκ, or variants or derivatives thereof.

In some embodiments, the IFN is a subtype of IFNα. In some embodiments, the IFNα is IFNα1, IFNα2, IFNα4, IFNα5, IFNα6, IFNα7, IFNα8, IFNα10, IFNα13, IFNα14, IFNα16, IFNα17, IFNα21, and derivatives thereof.

In some embodiments, the therapeutic polypeptide comprises one or more interferons. In some embodiments, the therapeutic polypeptide comprises IFNα, IFNβ, IFNγ, IFNα-IFNβ, IFNα-IFNγ, IFNβ-IFNγ, and IFNα-IFNβ-IFNγ. In some embodiments, the therapeutic polypeptide comprises IFNα, IFNβ, IFNγ, IFNα-IFNβ, IFNα-IFNγ, IFNβ-IFNγ, or IFNα-IFNβ-IFNγ, wherein the IFN is mouse. In some embodiments, the therapeutic polypeptide comprises IFNα, IFNβ, IFNγ, IFNα-IFNβ, IFNα-IFNγ, IFNβ-IFNγ, or IFNα-IFNβ-IFNγ, wherein the IFN is human. In some embodiments, the therapeutic polypeptide comprises IFNα, IFNβ, IFNγ, IFNα-IFNβ, IFNα-IFNγ, IFNβ-IFNγ, or IFNα-IFNβ-IFNγ, wherein the IFN is canine. In some embodiments, the therapeutic polypeptide comprises IFNα, IFNβ, IFNγ, IFNα-IFNβ, IFNα-IFNγ, IFNβ-IFNγ, or IFNα-IFNβ-IFNγ, wherein the IFN is rat. In some embodiments, the therapeutic polypeptide comprises IFNα, IFNβ, IFNγ, IFNα-IFNβ, IFNα-IFNγ, IFNβ-IFNγ, or IFNα-IFNβ-IFNγ, wherein the IFN is guinea pig.

In some embodiments, the human interferon beta (hIFNβ) has the sequence represented by UniProt/SwissProt Database Entry No. P01574 (SEQ ID NO: 1). In some embodiments, the hIFNβ is encoded by the nucleic acid sequence of any one of SEQ ID NOs: 2-4. In some embodiments, the hIFNβ is encoded by the nucleic acid sequence having at least 60% (e.g., 60%, 65%, 70%, 75%, 80%, 90%, 95%, 97%, 98%, or 99%) sequence identity to the nucleic acid sequence of any one of SEQ ID NOs: 2-4. In some embodiments, the hIFNβ is encoded by the nucleic acid sequence having at least 75% sequence identity to the nucleic acid sequence of any one of SEQ ID NOs: 2-4. In some embodiments, the hIFNβ is encoded by the nucleic acid sequence having at least 80% sequence identity to the nucleic acid sequence of any one of SEQ ID NOs: 2-4. In some embodiments, the hIFNβ is encoded by the nucleic acid sequence having at least 90% sequence identity to the nucleic acid sequence of any one of SEQ ID NOs: 2-4. In some embodiments, the hIFNβ is encoded by the nucleic acid sequence having at least 95% sequence identity to the nucleic acid sequence of any one of SEQ ID NOs: 2-4. In some embodiments, the hIFNβ is encoded by the nucleic acid sequence having at least 97% sequence identity to the nucleic acid sequence of any one of SEQ ID NOs: 2-4. In some embodiments, the hIFNβ is encoded by the nucleic acid sequence having at least 98% sequence identity to the nucleic acid sequence of any one of SEQ ID NOs: 2-4. In some embodiments, the hIFNβ is encoded by the nucleic acid sequence having at least 99% sequence identity to the nucleic acid sequence of any one of SEQ ID NOs: 2-4. In some embodiments, the hIFNβ is encoded by the nucleic acid sequence having the nucleic acid sequence of any one of SEQ ID NOs: 2-4.

In some embodiments, an interferon is a human IFNβ. In some embodiments, a human IFNβ comprises an amino acid sequence with at least 80%, at least 85%, at least 90%, at least 95%, at least 96%, at least 97%, at least 98%, or at least 99% sequence identity to SEQ ID NO: 1. In some embodiments, a human IFNβ comprises an amino acid sequence as set forth in SEQ ID NO: 1. In some embodiments, a human IFNβ is encoded by a polynucleotide sequence with at least 80%, at least 85%, at least 90%, at least 95%, at least 96%, at least 97%, at least 98%, or at least 99% sequence identity to SEQ ID NO: 2, SEQ ID NO: 3, or SEQ ID NO: 4. In some embodiments, a human IFNβ is encoded by a polynucleotide sequence as set forth in SEQ ID NO: 2, SEQ ID NO: 3, or SEQ ID NO: 4.

In some embodiments, the human interferon alpha 1 (hIFNα1) has the sequence represented by UniProt/SwissProt Database Entry No. P01562 (SEQ ID NO: 5). In some embodiments, the hIFNα is encoded by the nucleic acid sequence of any one of SEQ ID NOs: 6-8. In some embodiments, the hIFNα is encoded by the nucleic acid sequence having at least 60% (e.g., 60%, 65%, 70%, 75%, 80%, 90%, 95%, 97%, 98%, or 99%) sequence identity to the nucleic acid sequence of any one of SEQ ID NOs: 6-8. In some embodiments, the hIFNα is encoded by the nucleic acid sequence having at least 75% sequence identity to the nucleic acid sequence of any one of SEQ ID NOs: 6-8. In some embodiments, the hIFNα is encoded by the nucleic acid sequence having at least 80% sequence identity to the nucleic acid sequence of any one of SEQ ID NOs: 6-8. In some embodiments, the hIFNα is encoded by the nucleic acid sequence having at least 90% sequence identity to the nucleic acid sequence of any one of SEQ ID NOs: 6-8. In some embodiments, the hIFNα is encoded by the nucleic acid sequence having at least 95% sequence identity to the nucleic acid sequence of any one of SEQ ID NOs: 6-8. In some embodiments, the hIFNα is encoded by the nucleic acid sequence having at least 97% sequence identity to the nucleic acid sequence of any one of SEQ ID NOs: 6-8. In some embodiments, the hIFNα is encoded by the nucleic acid sequence having at least 98% sequence identity to the nucleic acid sequence of any one of SEQ ID NOs: 6-8. In some embodiments, the hIFNα is encoded by the nucleic acid sequence having at least 99% sequence identity to the nucleic acid sequence of any one of SEQ ID NOs: 6-8. In some embodiments, the hIFNα is encoded by the nucleic acid sequence having the nucleic acid sequence of any one of SEQ ID NOs: 6-8.

In some embodiments, an interferon is a human IFNα. In some embodiments, a human IFNα comprises an amino acid sequence with at least 80%, at least 85%, at least 90%, at least 95%, at least 96%, at least 97%, at least 98%, or at least 99% sequence identity to SEQ ID NO: 5. In some embodiments, a human IFNα comprises an amino acid sequence as set forth in SEQ ID NO: 5. In some embodiments, a human IFNα is encoded by a polynucleotide sequence with at least 80%, at least 85%, at least 90%, at least 95%, at least 96%, at least 97%, at least 98%, or at least 99% sequence identity to SEQ ID NO: 6, SEQ ID NO: 7, or SEQ ID NO: 8.

In some embodiments, a human IFNα is encoded by a polynucleotide sequence with at least 80%, at least 85%, at least 90%, at least 95%, at least 96%, at least 97%, at least 98%, or at least 99% sequence identity to SEQ ID NO: 6. In some embodiments, a human IFNα is encoded by a polynucleotide sequence with at least 80%, at least 85%, at least 90%, at least 95%, at least 96%, at least 97%, at least 98%, or at least 99% sequence identity to SEQ ID NO: 7. In some embodiments, a human IFNα is encoded by a polynucleotide sequence with at least 80%, at least 85%, at least 90%, at least 95%, at least 96%, at least 97%, at least 98%, or at least 99% sequence identity to SEQ ID NO: 8. In some embodiments, a human IFNα is encoded by a polynucleotide sequence as set forth in SEQ ID NO: 6, SEQ ID NO: 7, or SEQ ID NO: 8. In some embodiments, a human IFNα is encoded by a polynucleotide sequence as set forth in SEQ ID NO: 6. In some embodiments, a human IFNα is encoded by a polynucleotide sequence as set forth in SEQ ID NO: 7. In some embodiments, a human IFNα is encoded by a polynucleotide sequence as set forth in SEQ ID NO: 8.

In some embodiments, the human interferon gamma (hIFNγ) has the sequence represented by UniProt/SwissProt Database Entry No. P01579 (SEQ ID NO: 9). In some embodiments, the hIFNγ is encoded by the nucleic acid sequence of SEQ ID NOs: 10-12. In some embodiments, the hIFNγ is encoded by the nucleic acid sequence having at least 60% (e.g., 60%, 65%, 75%, 80%, 90%, 95%, 97%, 98%, or 99%) sequence identity to the nucleic acid sequence of SEQ ID NOs: 10-12. In some embodiments, the hIFNγ is encoded by the nucleic acid sequence having at least 75% sequence identity to the nucleic acid sequence of any one of SEQ ID NOs: 10-12. In some embodiments, the hIFNγ is encoded by the nucleic acid sequence having at least 80% sequence identity to the nucleic acid sequence of any one of SEQ ID NOs: 10-12. In some embodiments, the hIFNγ is encoded by the nucleic acid sequence having at least 90% sequence identity to the nucleic acid sequence of any one of SEQ ID NOs: 10-12. In some embodiments, the hIFNγ is encoded by the nucleic acid sequence having at least 95% sequence identity to the nucleic acid sequence of any one of SEQ ID NOs: 10-12. In some embodiments, the hIFNγ is encoded by the nucleic acid sequence having at least 97% sequence identity to the nucleic acid sequence of any one of SEQ ID NOs: 10-12. In some embodiments, the hIFNγ is encoded by the nucleic acid sequence having at least 98% sequence identity to the nucleic acid sequence of any one of SEQ ID NOs: 10-12. In some embodiments, the hIFNγ is encoded by the nucleic acid sequence having at least 99% sequence identity to the nucleic acid sequence of any one of SEQ ID NOs: 10-12. In some embodiments, the hIFNγ is encoded by the nucleic acid sequence having the nucleic acid sequence of any one of SEQ ID NOs: 10-12.

In some embodiments, an interferon is a human IFNγ. In some embodiments, a human IFNγ comprises an amino acid sequence with at least 80%, at least 85%, at least 90%, at least 95%, at least 96%, at least 97%, at least 98%, or at least 99% sequence identity to SEQ ID NO: 9. In some embodiments, a human IFNγ comprises an amino acid sequence as set forth in SEQ ID NO: 9. In some embodiments, a human IFNγ is encoded by a polynucleotide sequence with at least 80%, at least 85%, at least 90%, at least 95%, at least 96%, at least 97%, at least 98%, or at least 99% sequence identity to SEQ ID NO: 10, SEQ ID NO: 11, or SEQ ID NO: 12. In some embodiments, a human IFNγ is encoded by a polynucleotide sequence with at least 80%, at least 85%, at least 90%, at least 95%, at least 96%, at least 97%, at least 98%, or at least 99% sequence identity to SEQ ID NO: 10. In some embodiments, a human IFNγ is encoded by a polynucleotide sequence with at least 80%, at least 85%, at least 90%, at least 95%, at least 96%, at least 97%, at least 98%, or at least 99% sequence identity to SEQ ID NO: 11. In some embodiments, a human IFNγ is encoded by a polynucleotide sequence with at least 80%, at least 85%, at least 90%, at least 95%, at least 96%, at least 97%, at least 98%, or at least 99% sequence identity to SEQ ID NO: 12. In some embodiments, a human IFNγ is encoded by a polynucleotide sequence as set forth in SEQ ID NO: 10. In some embodiments, a human IFNγ is encoded by a polynucleotide sequence as set forth in SEQ ID NO: 11. In some embodiments, a human IFNγ is encoded by a polynucleotide sequence as set forth in SEQ ID NO: 12.

In some embodiments, the interferon is mouse interferon beta (mIFNβ). In some embodiments, the mouse interferon beta (mIFNβ) has the sequence represented by UniProt/SwissProt Database Entry No. P01575 (SEQ ID NO: 13). In some embodiments, the mIFNβ is encoded by the nucleic acid sequence of SEQ ID NO: 14 or 42. In some embodiments, the mIFNβ is encoded by the nucleic acid sequence having at least 60% (e.g., 60%, 65%, 75%, 80%, 90%, 95%, 97%, 98%, or 99%) sequence identity to the nucleic acid sequence of SEQ ID NO: 14 or 42. In some embodiments, the mIFNβ is encoded by the nucleic acid sequence having at least 75% sequence identity to the nucleic acid sequence of SEQ ID NO: 14 or 42. In some embodiments, the mIFNβ is encoded by the nucleic acid sequence having at least 80% sequence identity to the nucleic acid sequence of SEQ ID NO: 14 or 42. In some embodiments, the mIFNβ is encoded by the nucleic acid sequence having at least 90% sequence identity to the nucleic acid sequence of SEQ ID NO: 14 or 42. In some embodiments, the mIFNβ is encoded by the nucleic acid sequence having at least 95% sequence identity to the nucleic acid sequence of SEQ ID NO: 14 or 42. In some embodiments, the mIFNβ is encoded by the nucleic acid sequence having at least 97% sequence identity to the nucleic acid sequence of SEQ ID NO: 14 or 42. In some embodiments, the mIFNβ is encoded by the nucleic acid sequence having at least 98% sequence identity to the nucleic acid sequence of SEQ ID NO: 14 or 42. In some embodiments, the mIFNβ is encoded by the nucleic acid sequence having at least 99% sequence identity to the nucleic acid sequence of SEQ ID NO: 14 or 42. In some embodiments, the mIFNβ is encoded by the nucleic acid sequence having the nucleic acid sequence of SEQ ID NO: 14 or 42.

In some embodiments, an interferon is a mouse IFNβ. In some embodiments, a mouse IFNβ comprises an amino acid sequence with at least 80%, at least 85%, at least 90%, at least 95%, at least 96%, at least 97%, at least 98%, or at least 99% sequence identity to SEQ ID NO: 13. In some embodiments, a mouse IFNβ comprises an amino acid sequence as set forth in SEQ ID NO: 13. In some embodiments, a mouse IFNβ is encoded by a polynucleotide sequence with at least 80%, at least 85%, at least 90%, at least 95%, at least 96%, at least 97%, at least 98%, or at least 99% sequence identity to SEQ ID NO: 14. In some embodiments, a mouse IFNβ is encoded by a polynucleotide sequence with at least 80%, at least 85%, at least 90%, at least 95%, at least 96%, at least 97%, at least 98%, or at least 99% sequence identity to SEQ ID NO: 42. In some embodiments, a mouse IFNβ is encoded by a polynucleotide sequence as set forth in SEQ ID NO: 14. In some embodiments, a mouse IFNβ is encoded by a polynucleotide sequence as set forth in SEQ ID NO: 42.

In some embodiments, the interferon is canine interferon beta (cIFNβ). In some embodiments, the canine interferon beta (cIFNβ) has the sequence represented by UniProt/UniProtKB Database Entry No. B6E116 (SEQ ID NO: 15). In some embodiments, the interferon is canine interferon beta (cIFNβ) and is encoded by the nucleic acid sequence of SEQ ID NO: 16, 43, or 60. In some embodiments, the cIFNβ is encoded by the nucleic acid sequence having at least 60% (e.g., 60%, 65%, 75%, 80%, 90%, 95%, 97%, 98%, or 99%) sequence identity to the nucleic acid sequence of SEQ ID NO: 16, 43, or 60. In some embodiments, the cIFNβ is encoded by the nucleic acid sequence having at least 75% sequence identity to the nucleic acid sequence of SEQ ID NO: 16, 43, or 60. In some embodiments, the cIFNβ is encoded by the nucleic acid sequence having at least 80% sequence identity to the nucleic acid sequence of SEQ ID NO: 16, 43, or 60. In some embodiments, the cIFNβ is encoded by the nucleic acid sequence having at least 90% sequence identity to the nucleic acid sequence of SEQ ID NO: 16, 43, or 60. In some embodiments, the cIFNβ is encoded by the nucleic acid sequence having at least 95% sequence identity to the nucleic acid sequence of SEQ ID NO: 16, 43, or 60. In some embodiments, the cIFNβ is encoded by the nucleic acid sequence having at least 97% sequence identity to the nucleic acid sequence of SEQ ID NO: 16, 43, or 60. In some embodiments, the cIFNβ is encoded by the nucleic acid sequence having at least 98% sequence identity to the nucleic acid sequence of SEQ ID NO: 16, 43, or 60. In some embodiments, the cIFNβ is encoded by the nucleic acid sequence having at least 99% sequence identity to the nucleic acid sequence of SEQ ID NO: 16, 43, or 60. In some embodiments, the cIFNβ is encoded by the nucleic acid sequence having the nucleic acid sequence of SEQ ID NO: 16, 43, or 60.

In some embodiments, an interferon is a canine IFNβ. In some embodiments, a canine IFNβ comprises an amino acid sequence with at least 80%, at least 85%, at least 90%, at least 95%, at least 96%, at least 97%, at least 98%, or at least 99% sequence identity to SEQ ID NO: 15. In some embodiments, a canine IFNβ comprises an amino acid sequence as set forth in SEQ ID NO: 15. In some embodiments, a canine IFNβ is encoded by a polynucleotide sequence with at least 80%, at least 85%, at least 90%, at least 95%, at least 96%, at least 97%, at least 98%, or at least 99% sequence identity to SEQ ID NO: 16. In some embodiments, a canine IFNβ is encoded by a polynucleotide sequence with at least 80%, at least 85%, at least 90%, at least 95%, at least 96%, at least 97%, at least 98%, or at least 99% sequence identity to SEQ ID NO: 43. In some embodiments, a canine IFNβ is encoded by a polynucleotide sequence with at least 80%, at least 85%, at least 90%, at least 95%, at least 96%, at least 97%, at least 98%, or at least 99% sequence identity to SEQ ID NO: 60. In some embodiments, a canine IFNβ is encoded by a polynucleotide sequence as set forth in SEQ ID NO: 16. In some embodiments, a canine IFNβ is encoded by a polynucleotide sequence as set forth in SEQ ID NO: 43. In some embodiments, a canine IFNβ is encoded by a polynucleotide sequence as set forth in SEQ ID NO: 60.

In some embodiments, an interferon is a rat IFNβ. In some embodiments, a rat IFNβ comprises an amino acid sequence with at least 80%, at least 85%, at least 90%, at least 95%, at least 96%, at least 97%, at least 98%, or at least 99% sequence identity to SEQ ID NO: 61. In some embodiments, a rat IFNβ comprises an amino acid sequence as set forth in SEQ ID NO: 61.

In some embodiments, a rat IFNβ is encoded by a polynucleotide sequence with at least 80%, at least 85%, at least 90%, at least 95%, at least 96%, at least 97%, at least 98%, or at least 99% sequence identity to SEQ ID NO: 62. In some embodiments, a rat IFNβ is encoded by a polynucleotide sequence with at least 80%, at least 85%, at least 90%, at least 95%, at least 96%, at least 97%, at least 98%, or at least 99% sequence identity to SEQ ID NO: 63. In some embodiments, a rat IFNβ is encoded by a polynucleotide sequence as set forth in SEQ ID NO: 62. In some embodiments, a rat IFNβ is encoded by a polynucleotide sequence as set forth in SEQ ID NO: 63.

In some embodiments, an interferon is a guinea pig IFNβ. In some embodiments, a guinea pig IFNβ comprises an amino acid sequence with at least 80%, at least 85%, at least 90%, at least 95%, at least 96%, at least 97%, at least 98%, or at least 99% sequence identity to SEQ ID NO: 71. In some embodiments, a guinea pig IFNβ comprises an amino acid sequence as set forth in SEQ ID NO: 71.

In some embodiments, a guinea pig IFNβ is encoded by a polynucleotide sequence with at least 80%, at least 85%, at least 90%, at least 95%, at least 96%, at least 97%, at least 98%, or at least 99% sequence identity to SEQ ID NO: 72. In some embodiments, a guinea pig IFNβ is encoded by a polynucleotide sequence as set forth in SEQ ID NO: 72.

In some embodiments, the polynucleotide encoding for the interferon is codon optimized. In some embodiments, the polynucleotide encoding for the interferon is codon optimized for expression of the interferon. Codon optimization can be used to increase the rate of translation or to produce recombinant RNA transcripts having desirable properties, such greater expression efficiency, as compared with transcripts produced using a non-optimized sequence. In particular embodiments, the polynucleotide encoding for the interferon is codon optimized for expression in mammalian and human cells.

In some embodiments, provided herein are recombinant adeno-associated viral (rAAV) vectors comprising a polynucleotide encoding a payload of interest (e.g., a cytokine, such as any interferon described herein), where the coding sequence of said payload of interest comprises modified CpG dinucleotides (e.g., reduced, depleted, and/or methylation is increased) as compared to a parental equivalent. In one non-limiting example, the rAAV vectors comprise a polynucleotide encoding an interferon (e.g., IFNα, IFNβ, IFNγ, IFNε, IFNκ, IFNω, IFNλ, IFNχ, IFNξ, IFNτ, IFNδ, IFNν, IFNζ, IFN alfa, or variant or derivative thereof), where the coding sequence of said interferon comprises modified CpG dinucleotides (e.g., reduced, depleted, and/or methylation is increased) as compared to a parental equivalent. In some embodiments, an interferon coding polynucleotide comprises reduced CpG dinucleotides and/or increased methylation of CpG dinucleotides as compared to a parental equivalent. In some embodiments, CpG dinucleotides are completely methylated. In some embodiments, CpG dinucleotides are depleted.

In some embodiments, a polynucleotide encoding a payload of interest comprises reduced CpG dinucleotides as compared to a parental equivalent. In some embodiments, CpG dinucleotides are reduced by at least about 5%, 10%, 15%, 20%, 25%, 30%, 35%, 40%, 45%, 50%, 55%, 60%, 65%, 70%, 75%, 80%, 85%, 90%, 95%, or more than 99% as compared to a parental equivalent. In some embodiments, CpG dinucleotides are reduced in a range of about 5% to about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 10% to about 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 15% to about 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 20% to about 25%, 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 25% to about 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 30% to about 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 40% to about 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 50% to about 55%, 60%, 65%, 70%, 75%, 80%, 85%, 90%, 95%, 96%, 97%, 98%, 99%, or 100%. In some embodiments, CpG dinucleotides are reduced in a range of about 60% to about 65%, 70%, 75%, 80%, 85%, 90%, 95%, 96%, 97%, 98%, 99%, or 100%. In some embodiments, CpG dinucleotides are reduced in a range of about 70% to about 75%, 80%, 85%, 90%, 95%, 96%, 97%, 98%, 99%, or 100%. In some embodiments, CpG dinucleotides are reduced in a range of about 80% to about 85%, 90%, 95%, 96%, 97%, 98%, 99%, or 100%. In some embodiments, CpG dinucleotides are reduced in a range of about 90% to about 95%, 96%, 97%, 98%, 99%, or 100%. In some embodiments, CpG dinucleotides are reduced by at least about 50%. In some embodiments, CpG dinucleotides are reduced by at least about 75%.

In some embodiments, a polynucleotide encoding a payload of interest comprises depleted CpG dinucleotides as compared to a parental equivalent.

In some embodiments, CpG dinucleotides are reduced by deletion of certain CpG dinucleotides. In some embodiments, CpG dinucleotides are reduced by substitution of one or more nucleotides within a CpG dinucleotide to create a sequence that is not a CpG dinucleotide. In some embodiments, CpG dinucleotides are depleted by deletion of all CpG dinucleotides. In some embodiments, CpG dinucleotides are depleted by substitution of one or more nucleotides within each CpG dinucleotide to create sequences that are not CpG dinucleotides.

In some embodiments, a polynucleotide encoding a payload of interest comprises increased methylation of CpG dinucleotides as compared to a parental equivalent. In some embodiments, methylation of CpG dinucleotides is increased by at least about 5%, 10%, 15%, 20%, 25%, 30%, 35%, 40%, 45%, 50%, 55%, 60%, 65%, 70%, 75%, 80%, 85%, 90%, 95%, or more than 95% as compared to a parental equivalent. In some embodiments, methylation of CpG dinucleotides is increased in a range of about 5% to about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 10% to about 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 15% to about 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 20% to about 25%, 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 25% to about 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 30% to about 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 40% to about 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 50% to about 55%, 60%, 65%, 70%, 75%, 80%, 85%, 90%, 95%, 96%, 97%, 98%, 99%, or 100%. In some embodiments, methylation of CpG dinucleotides is increased in a range of about 60% to about 65%, 70%, 75%, 80%, 85%, 90%, 95%, 96%, 97%, 98%, 99%, or 100%. In some embodiments, methylation of CpG dinucleotides is increased in a range of about 70% to about 75%, 80%, 85%, 90%, 95%, 96%, 97%, 98%, 99%, or 100%. In some embodiments, methylation of CpG dinucleotides is increased in a range of about 80% to about 85%, 90%, 95%, 96%, 97%, 98%, 99%, or 100%. In some embodiments, methylation of CpG dinucleotides is increased in a range of about 90% to about 95%, 96%, 97%, 98%, 99%, or 100%.

In some embodiments, a polynucleotide encoding an interferon (e.g., a mouse, human, canine, feline, ruminant, rat, guinea pig, primate, pig, or ferret IFNα, IFNβ, IFNγ, IFNε, IFNκ, IFNω, IFNλ, IFNχ, IFNξ, IFNτ, IFNδ, IFNν, IFNζ, IFN alfa, including variants or derivatives thereof) comprises reduced CpG dinucleotides as compared to a parental equivalent. In some embodiments, CpG dinucleotides are reduced by at least about 5%, 10%, 15%, 20%, 25%, 30%, 35%, 40%, 45%, 50%, 55%, 60%, 65%, 70%, 75%, 80%, 85%, 90%, 95%, or more than 99% as compared to a parental equivalent. In some embodiments, CpG dinucleotides are reduced in a range of about 5% to about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 10% to about 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 15% to about 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 20% to about 25%, 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 25% to about 30%, 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 30% to about 35%, 40%, 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 40% to about 45%, 50%, 55%, 60%, 65%, 70%, 75%, 80%, 85%, 90%, 95%, 96%, 97%, 98%, 99%, or 100%. In some embodiments, CpG dinucleotides are reduced in a range of about 50% to about 55%, 60%, 65%, 70%, 75%, 80%, 85%, 90%, 95%, 96%, 97%, 98%, 99%, or 100%. In some embodiments, CpG dinucleotides are reduced in a range of about 60% to about 65%, 70%, 75%, 80%, 85%, 90%, 95%, 96%, 97%, 98%, 99%, or 100%. In some embodiments, CpG dinucleotides are reduced in a range of about 70% to about 75%, 80%, 85%, 90%, 95%, 96%, 97%, 98%, 99%, or 100%. In some embodiments, CpG dinucleotides are reduced in a range of about 80% to about 85%, 90%, 95%, 96%, 97%, 98%, 99%, or 100%. In some embodiments, CpG dinucleotides are reduced in a range of about 90% to about 95%, 96%, 97%, 98%, 99%, or 100%. In some embodiments, CpG dinucleotides are reduced by at least about 50%. In some embodiments, CpG dinucleotides are reduced by at least about 75%.

In some embodiments, a polynucleotide encoding an interferon comprises depleted CpG dinucleotides as compared to a parental equivalent.

In some embodiments, CpG dinucleotides are reduced by deletion of certain CpG dinucleotides. In some embodiments, CpG dinucleotides are reduced by substitution of one or more nucleotides within a CpG dinucleotide to create a sequence that is not a CpG dinucleotide. In some embodiments, CpG dinucleotides are depleted by deletion of all CpG dinucleotides. In some embodiments, CpG dinucleotides are depleted by substitution of one or more nucleotides within each CpG dinucleotide to create sequences that are not CpG dinucleotides.

In some embodiments, a polynucleotide encoding an interferon comprises increased methylation of CpG dinucleotides as compared to a parental equivalent. In some embodiments, methylation of CpG dinucleotides is increased by at least about 5%, 10%, 15%, 20%, 25%, 30%, 35%, 40%, 45%, 50%, 55%, 60%, 65%, 70%, 75%, 80%, 85%, 90%, 95%, or more than 95% as compared to a parental equivalent. In some embodiments, methylation of CpG dinucleotides is increased in a range of about 5% to about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 10% to about 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 15% to about 20%, 25%, 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 20% to about 25%, 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 25% to about 30%, 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 30% to about 35%, 40%, 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 40% to about 45%, 50%, 55%, 60%, 65%, 70%, 75%, 80%, 85%, 90%, 95%, 96%, 97%, 98%, 99%, or 100%. In some embodiments, methylation of CpG dinucleotides is increased in a range of about 50% to about 55%, 60%, 65%, 70%, 75%, 80%, 85%, 90%, 95%, 96%, 97%, 98%, 99%, or 100%. In some embodiments, methylation of CpG dinucleotides is increased in a range of about 60% to about 65%, 70%, 75%, 80%, 85%, 90%, 95%, 96%, 97%, 98%, 99%, or 100%. In some embodiments, methylation of CpG dinucleotides is increased in a range of about 70% to about 75%, 80%, 85%, 90%, 95%, 96%, 97%, 98%, 99%, or 100%. In some embodiments, methylation of CpG dinucleotides is increased in a range of about 80% to about 85%, 90%, 95%, 96%, 97%, 98%, 99%, or 100%. In some embodiments, methylation of CpG dinucleotides is increased in a range of about 90% to about 95%, 96%, 97%, 98%, 99%, or 100%.

In some embodiments, the rAAV vectors described herein comprise one or more polynucleotides encoding one or more interferons. In some embodiments, the one or more polynucleotides encode polypeptides separated by a self-cleaving peptide. For example, the first polynucleotide encoding a first interferon polypeptide and the second polynucleotide encoding a second interferon polypeptide is connected by a polynucleotide encoding a first self-cleaving peptide. Optionally a sequence encoding a linker (e.g., Gly-Ser-Gly) is upstream (5′) from the sequence encoding the self-cleaving peptide. In some embodiments, the second polynucleotide encoding a second interferon polypeptide and the third polynucleotide encoding a third interferon polypeptide are connected by a polynucleotide encoding a second linker peptide and a polynucleotide encoding a second self-cleaving peptide. In some embodiments, the polynucleotide encoding a second self-cleaving peptide is upstream (3′) from the polynucleotide encoding a second linker peptide.

Suitable self-cleaving peptides include a 2A self-cleaving peptide, such as a P2A self-cleaving peptide, a T2A self-cleaving peptide, a F2A self-cleaving peptide, or an E2A self-cleaving peptide. In some embodiments, the self-cleaving peptide is a P2A self-cleaving peptide and has the sequence of SEQ ID NO: 17.

In some embodiments, a first self-cleaving peptide and a second self-cleaving peptide are the same. For example, in some embodiments, both are P2A. In some embodiments, a first self-cleaving peptide and a second self-cleaving peptide are not the same. For example, in some embodiments, a first self-cleaving peptide is a P2A self-cleaving peptide, and a second self-cleaving peptide is a T2A self-cleaving peptide. In some embodiments, the second self-cleaving peptide is a T2A self-cleaving peptide and has the sequence of SEQ ID NO: 18.

### Vectors and Viral Packaging

In some embodiments, the polynucleotide of the present disclosure (e.g., a polynucleotide encoding a payload of interest) is delivered by a vector. In some embodiments, the polynucleotide is delivered by a plasmid (e.g., circular DNA molecules that can autonomously replicate inside a cell), cosmid (e.g., pWE or sCos vectors), artificial chromosome, human artificial chromosome (HAC), yeast artificial chromosomes (YAC), bacterial artificial chromosome (BAC), minicircle, doggybone, nanoplasmid, P1-derived artificial chromosomes (PAC), phagemid, phage derivative, bacmid, or virus. In some embodiments, the vector is selected from the list consisting of: pMB1, pBR322, ColE1, R6K, p15A, pSC101, ColE2, F1, pUC, pBluescript, pSF-CMV-NEO-NH2-PPT-3×FLAG, pSF-CMV-NEO-COOH-3×FLAG, pSF-CMV-PURO-NH2-GST-TEV, pCEP4 pDEST27, pSF-CMV-Ub-KrYFP, pSF-CMV-FMDV-daGFP, pEF1a-mCherry-N1, pEF1a-tdTomato, pSF-CMV-FMDV-Hygro, pSF-CMV-PGK-Puro, pMCP-tag(m), and pSF-CMV-PURO-NH2-CMYC, pSF-OXB20-BetaGal, pSF-OXB20-Fluc, pSF-OXB20, pSF-Tac, pSF-OXB20-COOH-TEV-FLAG (R)-6His, pRI 101-AN DNA, pCambia2301, pTYB21, pKLAC2, pAc5.1/V5-His A, and pDEST8.

In some embodiments, the plasmids comprise the polynucleotide and an antibiotic resistance gene (e.g., any antibiotic resistance gene described herein). In some embodiments, the plasmids further comprise one or more regulatory elements (e.g., any of those described herein). In some embodiments, the one or more regulatory elements (e.g., a promoter, an enhancer, a WPRE, and/or a polyA, and so forth) are operably linked to one or more coding polynucleotides (e.g., polynucleotides that encode a payload of interest) in a plasmid.

In some embodiments, the polynucleotide of the present disclosure (e.g., a polynucleotide encoding a payload of interest) is delivered by a virus. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a poliovirus, a vaccinia virus, or a retrovirus. In some embodiments, the virus is an alphavirus. In some embodiments, the virus is a parvovirus. In some embodiments, the virus is an adenovirus. In some embodiments, the virus is an AAV. In some embodiments, the virus is a baculovirus. In some embodiments, the virus is a Dengue virus. In some embodiments, the virus is a lentivirus. In some embodiments, the virus is a herpesvirus. In some embodiments, the virus is a poxvirus. In some embodiments, the virus is an anellovirus. In some embodiments, the virus is a bocavirus. In some embodiments, the virus is a poliovirus. In some embodiments, the virus is a vaccinia virus. In some embodiments, the virus is a retrovirus.

In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof. In some embodiments, the herpesvirus is HSV type 1, HSV-2, VZV, EBV, CMV, HHV-6, HHV-7, or HHV-8.

In some embodiments, the AAV is AAV1 or a derivative thereof. In some embodiments, the AAV is AAV2 or a derivative thereof. In some embodiments, the AAV is AAV3 or a derivative thereof. In some embodiments, the AAV is AAV4 or a derivative thereof. In some embodiments, the AAV is AAV5 or a derivative thereof. In some embodiments, the AAV is AAV6 or a derivative thereof. In some embodiments, the AAV is AAV7 or a derivative thereof. In some embodiments, the AAV is AAV8 or a derivative thereof. In some embodiments, the AAV is AAV9 or a derivative thereof. In some embodiments, the AAV is AAV10 or a derivative thereof. In some embodiments, the AAV is AAV11 or a derivative thereof. In some embodiments, the AAV is AAV12 or a derivative thereof. In some embodiments, the AAV is AAV13 or a derivative thereof. In some embodiments, the AAV is AAV14 or a derivative thereof. In some embodiments, the AAV is AAV15 or a derivative thereof. In some embodiments, the AAV is AAV16 or a derivative thereof. In some embodiments, the AAV is AAV-rh8 or a derivative thereof. In some embodiments, the AAV is AAV-rh10 or a derivative thereof. In some embodiments, the AAV is AAV-rh20 or a derivative thereof. In some embodiments, the AAV is AAV-rh39 or a derivative thereof. In some embodiments, the AAV is AAV-rh74 or a derivative thereof. In some embodiments, the AAV is AAV-rhM4-1 or a derivative thereof. In some embodiments, the AAV is AAV-hu37 or a derivative thereof. In some embodiments, the AAV is AAV-Anc80 or a derivative thereof. In some embodiments, the AAV is AAV-Anc80L65 or a derivative thereof. In some embodiments, the AAV is AAV-7m8 or a derivative thereof. In some embodiments, the AAV is AAV-PHP-B or a derivative thereof. In some embodiments, the AAV is AAV-PHP-EB or a derivative thereof. In some embodiments, the AAV is AAV-2.5 or a derivative thereof. In some embodiments, the AAV is AAV-2tYF or a derivative thereof. In some embodiments, the AAV is AAV-3B or a derivative thereof. In some embodiments, the AAV is AAV-LK03 or a derivative thereof. In some embodiments, the AAV is AAV-HSC1 or a derivative thereof. In some embodiments, the AAV is AAV-HSC2 or a derivative thereof. In some embodiments, the AAV is AAV-HSC3 or a derivative thereof. In some embodiments, the AAV is AAV-HSC4 or a derivative thereof. In some embodiments, the AAV is AAV-HSC5 or a derivative thereof. In some embodiments, the AAV is AAV-HSC6 or a derivative thereof. In some embodiments, the AAV is AAV-HSC7 or a derivative thereof. In some embodiments, the AAV is AAV-HSC8 or a derivative thereof. In some embodiments, the AAV is AAV-HSC9 or a derivative thereof. In some embodiments, the AAV is AAV-HSC10 or a derivative thereof. In some embodiments, the AAV is AAV-HSC11 or a derivative thereof. In some embodiments, the AAV is AAV-HSC12 or a derivative thereof. In some embodiments, the AAV is AAV-HSC13 or a derivative thereof. In some embodiments, the AAV is AAV-HSC14 or a derivative thereof. In some embodiments, the AAV is AAV-HSC15 or a derivative thereof. In some embodiments, the AAV is AAV-TT or a derivative thereof. In some embodiments, the AAV is AAV-DJ/8 or a derivative thereof. In some embodiments, the AAV is AAV-Myo or a derivative thereof. In some embodiments, the AAV is AAV-NP40 or a derivative thereof. In some embodiments, the AAV is AAV-NP59 or a derivative thereof. In some embodiments, the AAV is AAV-NP22 or a derivative thereof. In some embodiments, the AAV is AAV-NP66 or a derivative thereof. In some embodiments, the AAV is AAV-HSC16 or a derivative thereof.

In some embodiments, the virus is HSV-1 or a derivative thereof. In some embodiments, the virus is HSV-2 or a derivative thereof. In some embodiments, the virus is VZV or a derivative thereof. In some embodiments, the virus is EBV or a derivative thereof. In some embodiments, the virus is CMV or a derivative thereof. In some embodiments, the virus is HHV-6 or a derivative thereof. In some embodiments, the virus is HHV-7 or a derivative thereof. In some embodiments, the virus is HHV-8 or a derivative thereof.

### Regulatory Elements

In some embodiments, recombinant adeno-associated virus (rAAV) vectors of the present disclosure comprise one or more regulatory elements. In some embodiments, the one or more regulatory elements is operably linked to a polynucleotide comprising a coding sequence (e.g., a coding sequence for a polypeptide (e.g., a therapeutic polypeptide, a reporter polypeptide, etc). In some embodiments, the regulatory element facilitates a particular function (e.g., RNA splicing, translation initiation, translation termination, etc.) during and/or after transcription of a mRNA transcript. In some embodiments, a regulatory element facilitates a particular function when present on a DNA template. In some embodiments, a regulatory element facilitates a particular function when present on a RNA template. In some embodiments, a regulatory element is selected from the group consisting of: a promoter, an enhancer, a terminator sequence, an mRNA stability sequence, a sequence that allows for internal ribosome entry sites (IRES) of bicistronic mRNA, a sequence that inhibits viral recognition (e.g., by Toll-like or RIG-like receptors such as TLR7, TLR8, TLR9, MDA5, RIG1, and/or DAI), a sequence necessary for transduction into a cell, an intron, a synthetic intron, an exon, a synthetic exon, and a polyA signal. In some embodiments, the rAAV vector comprises a promoter, an enhancer, an intron, a microRNA, a linker, a splicing element, a sequence that allows for internal ribosome entry sites (IRES) of bicistronic mRNA, a polyA signal sequence, or combinations thereof. In some embodiments, a sequence that allows for internal ribosome entry sites (IRES) of bicistronic mRNA is Woodchuck Hepatitis Virus (WHP) Posttranscriptional Regulatory Element (WPRE). In some embodiments, the rAAV comprises a promoter. In some embodiments, the rAAV vector comprises an enhancer. In some embodiments, the rAAV vector comprises an intron. In some embodiments, the rAAV vector comprises a synthetic intron. In some embodiments, the rAAV vector comprises a microRNA. In some embodiments, the rAAV vector comprises a linker. In some embodiments, the rAAV vector comprises a splicing element. In some embodiments, the rAAV vector comprises a polyA signal sequence.

In some embodiments, the polyA signal sequence is from SV40. In some embodiments, the polyA signal sequence comprises the nucleic acid sequence of SEQ ID NO: 19. In some embodiments, the polyA signal sequence is from bovine growth hormone (bGH). Examples of other suitable polyA signals include, a synthetic polyA signal, a polyA from human growth hormone (hGH), rabbit beta-globin (RGB), or modified RGB (mRGB).

In some embodiments, the polyA sequence is selected from the group consisting of: SV40, hGH, bGH, rbGlob, and derivatives and variants thereof. In some embodiments, a polyA sequence is a SV40 sequence, or a derivative or variant thereof. In some embodiments, a SV40 sequence comprises a polynucleotide as set forth in SEQ ID NO: 19. In some embodiments, a polyA sequence is a bGH sequence, or a derivative or variant thereof. In some embodiments, a bGH sequence comprises a polynucleotide sequence as set forth in SEQ ID NO: 34. In some embodiments, a polyA sequence is a bGH sequence, or a derivative or variant thereof. In some embodiments, a polyA sequence is a rbGlob sequence, or a derivative or variant thereof.

In some embodiments, the rAAV vector comprises a promoter. In some embodiments, the promoter is selected from the group consisting of: a mini promoter, an inducible promoter, a constitutive promoter, and derivatives thereof. In some embodiments, the constitutive promoter comprises a non-bacterial leader sequence. In some embodiments, the promoter is selected from the group consisting of: CMV, CBA, EF1a, CAG, PGK, TRE, U6, UAS, T7, Sp6, lac, araBad, trp, Ptac, p5, p19, p40, Synapsin, GFAP, CaMKII, GRK1, and derivatives thereof. In some embodiments, the promoter is the CMV promoter or a derivative thereof. In some embodiments, the promoter is the CBA promoter or a derivative thereof. In some embodiments, the promoter is the EF1a promoter or a derivative thereof. In some embodiments, the promoter is the CAG promoter or a derivative thereof. In some embodiments, the promoter is the PGK promoter or a derivative thereof. In some embodiments, the promoter is the TRE promoter or a derivative thereof. In some embodiments, the promoter is the U6 promoter or a derivative thereof. In some embodiments, the promoter is the UAS promoter or a derivative thereof. In some embodiments, the promoter is the T7 promoter or a derivative thereof. In some embodiments, the promoter is the Sp6 promoter or a derivative thereof. In some embodiments, the promoter is the lac promoter or a derivative thereof. In some embodiments, the promoter is the araBad promoter or a derivative thereof. In some embodiments, the promoter is the trp promoter or a derivative thereof. In some embodiments, the promoter is the Ptac promoter or a derivative thereof. In some embodiments, the promoter is the p5 promoter or a derivative thereof. In some embodiments, the promoter is the p19 promoter or a derivative thereof. In some embodiments, the promoter is the p40 promoter or a derivative thereof. In some embodiments, the promoter is the Synapsin promoter or a derivative thereof. In some embodiments, the promoter is the GFAP promoter or a derivative thereof. In some embodiments, the promoter is the CaMKII promoter or a derivative thereof. In some embodiments, the promoter is the GRK1 promoter or a derivative thereof. In some embodiments, the promoter is a mini promoter or a derivative thereof. In some embodiments, the promoter is an inducible promoter.

In some embodiments, the promoter is a CAG promoter. A CAG promoter is a synthetic promoter that is a hybrid of the cytomegalovirus (CMV) early enhancer element and chicken beta-actin (CBA) promoter which comprises (1) a CMV immediate early enhancer element, (2) a CBA promoter element, including the first exon and the first intron of CBA gene, and (3) a splice acceptor element of the rabbit beta-globin gene. In some embodiments, a CMV immediate early enhancer element is derived from a wild-type CMV enhancer. In some embodiments, a CMV immediate early enhancer element is truncated with respect to a wild-type CMV enhancer. In some embodiments, a CAG promoter comprises a polynucleotide with at least 80%, at least 85%, at least 90%, at least 95%, at least 96%, at least 97%, at least 98%, or at least 99% sequence identity to SEQ ID NO: 48. In some embodiments, a CAG promoter comprises a polynucleotide with at least 80%, at least 85%, at least 90%, at least 95%, at least 96%, at least 97%, at least 98%, or at least 99% sequence identity to SEQ ID NO: 49. In some embodiments, a CAG promoter comprises a polynucleotide with at least 80%, at least 85%, at least 90%, at least 95%, at least 96%, at least 97%, at least 98%, or at least 99% sequence identity to SEQ ID NO: 50. In some embodiments, a CAG promoter comprises a polynucleotide as set forth in SEQ ID NO: 48. In some embodiments, a CAG promoter comprises a polynucleotide as set forth in SEQ ID NO: 49. In some embodiments, a CAG promoter comprises a polynucleotide as set forth in SEQ ID NO: 50. In some embodiments, a CAG promoter comprises a polynucleotide with at least 80%, at least 85%, at least 90%, at least 95%, at least 96%, at least 97%, at least 98%, or at least 99% sequence identity to SEQ ID NO: 22. In some embodiments, a CAG promoter comprises a polynucleotide with at least 80%, at least 85%, at least 90%, at least 95%, at least 96%, at least 97%, at least 98%, or at least 99% sequence identity to SEQ ID NO: 23. In some embodiments, a CAG promoter comprises a polynucleotide with at least 80%, at least 85%, at least 90%, at least 95%, at least 96%, at least 97%, at least 98%, or at least 99% sequence identity to SEQ ID NO: 24. In some embodiments, a CAG promoter comprises a polynucleotide with at least 80%, at least 85%, at least 90%, at least 95%, at least 96%, at least 97%, at least 98%, or at least 99% sequence identity to SEQ ID NO: 64. In some embodiments, a CAG promoter comprises a polynucleotide with at least 80%, at least 85%, at least 90%, at least 95%, at least 96%, at least 97%, at least 98%, or at least 99% sequence identity to SEQ ID NO: 65.

In some embodiments, a CMV immediate early enhancer element comprises a polynucleotide with at least 80%, at least 85%, at least 90%, at least 95%, at least 96%, at least 97%, at least 98%, or at least 99% sequence identity to SEQ ID NO: 25.

In some embodiments, a CMV immediate early enhancer element comprises a polynucleotide with at least 80%, at least 85%, at least 90%, at least 95%, at least 96%, at least 97%, at least 98%, or at least 99% sequence identity to SEQ ID NO: 26. In some embodiments, a CMV early enhancer element comprises a polynucleotide sequence as set forth in SEQ ID NO: 26.

In some embodiments, the CMV immediate early enhancer element comprises a polynucleotide with at least 80%, at least 85%, at least 90%, at least 95%, at least 96%, at least 97%, at least 98%, or at least 99% sequence identity to SEQ ID NO: 27. In some embodiments, a CMV immediate early enhancer element comprises a polynucleotide sequence as set forth in SEQ ID NO: 27.

In some embodiments, the promoter element is derived from a chicken beta-actin gene. In some embodiments, a promoter element comprises a polynucleotide with at least 80%, at least 85%, at least 90%, at least 95%, at least 96%, at least 97%, at least 98%, or at least 99% sequence identity to SEQ ID NO: 28. In some embodiments, a promoter element comprises a polynucleotide sequence as set forth in SEQ ID NO: 28. In some embodiments, a promoter element comprises a polynucleotide with at least 80%, at least 85%, at least 90%, at least 95%, at least 96%, at least 97%, at least 98%, or at least 99% sequence identity to SEQ ID NO: 52. In some embodiments, a promoter element comprises a polynucleotide sequence as set forth in SEQ ID NO: 52.

In some embodiments, the splice acceptor is derived from a rabbit beta-globin gene. In some embodiments, a splice acceptor refers to an intron that comprises a splice acceptor polynucleotide sequence. In some embodiments, a splice acceptor comprises a polynucleotide with at least 80%, at least 85%, at least 90%, at least 95%, at least 96%, at least 97%, at least 98%, or at least 99% sequence identity to SEQ ID NO: 30. In some embodiments, a splice acceptor comprises a polynucleotide sequence as set forth in SEQ ID NO: 30. In some embodiments, a splice acceptor comprises a polynucleotide with at least 80%, at least 85%, at least 90%, at least 95%, at least 96%, at least 97%, at least 98%, or at least 99% sequence identity to SEQ ID NO: 53. In some embodiments, a splice acceptor comprises a polynucleotide sequence as set forth in SEQ ID NO: 53.

In some embodiments, the polynucleotide sequence that allows for internal ribosome entry sites (IRES) of bicistronic mRNA is a WPRE. In some embodiments, a WPRE sequence is a wild-type WPRE. In some embodiments, a WPRE comprises a nucleic acid sequence with at least 80%, at least 85%, at least 90%, at least 95%, at least 96%, at least 97%, at least 98%, or at least 99% sequence identity to SEQ ID NO: 32. In some embodiments, a WPRE comprises a nucleic acid sequence as set forth in SEQ ID NO: 32. In some embodiments, a WPRE sequence is a modified WPRE sequence. In some embodiments, a modified WPRE comprises a nucleic acid sequence with at least 80%, at least 85%, at least 90%, at least 95%, at least 96%, at least 97%, at least 98%, or at least 99% sequence identity to SEQ ID NO: 33. In some embodiments, a modified WPRE comprises an amino acid sequence as set forth in SEQ ID NO: 33.

### Inverted Terminal Repeat Sequences

Inverted terminal repeat (ITR) sequences typically comprise 145 bases each (i.e., both 5′ and 3′ ITR sequences comprises 145 bases each).

In some embodiments, the recombinant adeno-associated virus (rAAV) vectors described herein further comprises a first ITR sequence and a second ITR sequence. In some embodiments, the rAAV vector comprises, from 5′ to 3′: a) a first ITR sequence; b) a promoter; c) a polynucleotide encoding a payload of interest; and d) a second ITR sequence. In some embodiments, a nucleic acid comprises, from 5′ to 3′: a) a first ITR sequence; b) a promoter; c) a minigene comprising a splice modulator site; d) a polynucleotide encoding a payload of interest; and e) a second ITR sequence.

In some embodiments, a first ITR sequence and/or a second ITR sequence are truncated as compared to the corresponding wild-type ITR sequence, or a parental equivalent. In some embodiments, a first ITR sequence and/or a second ITR sequence are truncated by at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides at the 5′ end or 3′ end. In some embodiments, a first ITR sequence is truncated by about 5 nucleotides at the 5′ end. In some embodiments, a first ITR sequence is truncated by about 10 nucleotides at the 5′ end. In some embodiments, a first ITR sequence is truncated by about 15 nucleotides at the 5′ end. In some embodiments, a first ITR sequence is truncated by about 20 nucleotides at the 5′ end. In some embodiments, a first ITR sequence is truncated by about 25 nucleotides at the 5′ end. In some embodiments, a first ITR sequence is truncated by about 30 nucleotides at the 5′ end. In some embodiments, a first ITR sequence is truncated by 15 nucleotides at the 5′ end. In some embodiments, a second ITR sequence is truncated by about 5 nucleotides at the 3′ end. In some embodiments, a second ITR sequence is truncated by about 10 nucleotides at the 3′ end. In some embodiments, a second ITR sequence is truncated by about 15 nucleotides at the 3′ end. In some embodiments, a second ITR sequence is truncated by about 20 nucleotides at the 3′ end. In some embodiments, a second ITR sequence is truncated by about 25 nucleotides at 3′ end. In some embodiments, a second ITR sequence is truncated by about 30 nucleotides at the 3′ end. In some embodiments, a second ITR sequence is truncated by 15 nucleotides at the 3′ end. In some embodiments, the first ITR sequence is modified to promote the formation of a self-complementary AAV genome (scAAV). In some embodiments, the second ITR sequence is modified to promote the formation of a self-complementary AAV genome.

In some embodiments, a first ITR sequence and/or a second ITR sequence comprise an ITR derived from an AAV serotype selected from the group consisting of: AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof.

In some embodiments, a first ITR sequence and/or a second ITR sequence are, or are derived from, an AAV2 ITR sequence. In some embodiments, a first ITR sequence is, or is derived from, an AAV2 ITR sequence. In some embodiments, a second ITR sequence is, or is derived from, an AAV2 ITR sequence.

In some embodiments, a first ITR sequence and/or a second ITR sequence comprise a nucleic acid sequence with at least 75%, 80%, 85%, 90%, 95%, or 99% sequence identity to SEQ ID NO: 36, SEQ ID NO: 37, SEQ ID NO: 38, SEQ ID NO: 39, SEQ ID NO: 40, or SEQ ID NO: 41. In some embodiments, a first ITR sequence and/or a second ITR sequence comprise a nucleic acid sequence as set forth in SEQ ID NO: 36, SEQ ID NO: 37, SEQ ID NO: 38, SEQ ID NO: 39, SEQ ID NO: 40, or SEQ ID NO: 41. In some embodiments, a first ITR sequence comprises a nucleic acid sequence as set forth in SEQ ID NO: 36. In some embodiments, a first ITR sequence comprises a nucleic acid sequence as set forth in SEQ ID NO: 37. In some embodiments, a first ITR sequence comprises a nucleic acid sequence as set forth in SEQ ID NO: 38. In some embodiments, a first ITR sequence comprises a nucleic acid sequence as set forth in SEQ ID NO: 39. In some embodiments, a first ITR sequence comprises a nucleic acid sequence as set forth in SEQ ID NO: 40. In some embodiments, a first ITR sequence comprises a nucleic acid sequence as set forth in SEQ ID NO: 41. In some embodiments, a second ITR sequence comprise a nucleic acid sequence as set forth in SEQ ID NO: 36. In some embodiments, a second ITR sequence comprise a nucleic acid sequence as set forth in SEQ ID NO: 37. In some embodiments, a second ITR sequence comprise a nucleic acid sequence as set forth in SEQ ID NO: 38. In some embodiments, a second ITR sequence comprise a nucleic acid sequence as set forth in SEQ ID NO: 39. In some embodiments, a second ITR sequence comprise a nucleic acid sequence as set forth in SEQ ID NO: 40. In some embodiments, a second ITR sequence comprise a nucleic acid sequence as set forth in SEQ ID NO: 41.

### Additional Nucleic Acid Elements

In some embodiments, recombinant adeno-associated virus (rAAV) vectors described herein further comprises an antibiotic resistance gene, an origin of replication, an open reading frame, or combinations thereof. In some embodiments, the rAAV vector comprises an antibiotic resistance gene, an origin of replication, and an open reading frame.

In some embodiments, the rAAV vectors further comprise an antibiotic resistance gene. In some embodiments, the antibiotic resistance gene encodes for resistance to an aminoglycoside, a beta-lactam, a macrolide, a tetracycline, or derivatives thereof. In some embodiments, the rAAV vector comprises an antibiotic resistance gene for aminoglycoside or a derivative thereof. In some embodiments, the rAAV vector comprises an antibiotic resistance gene for a beta-lactam or a derivative thereof. In some embodiments, the rAAV vector comprises an antibiotic resistance gene for a macrolide or a derivative thereof. In some embodiments, the rAAV vector comprises an antibiotic resistance gene conferring resistance to a tetracycline or a derivative thereof.

In some embodiments, the antibiotic resistance gene confers resistance to kanamycin, spectinomycin, streptomycin, ampicillin, carbenicillin, bleomycin, erythromycin, polymyxin B, tetracycline, chloramphenicol, neomycin, zeocin, or derivatives thereof.

In some embodiments, an ampicillin resistance gene comprises a nucleotide sequence having at least 70% (e.g., 75%, 80%, 90%, 95%, 97%, 98%, or 99%) sequence identity to the nucleic acid sequence of SEQ ID NO: 44. In some embodiments, the ampicillin resistance gene comprises a nucleotide sequence having at least 75% sequence identity to the nucleic acid sequence of SEQ ID NO: 44. In some embodiments, the ampicillin resistance gene comprises a nucleotide sequence having at least 80% sequence identity to the nucleic acid sequence of SEQ ID NO: 44. In some embodiments, the ampicillin resistance gene comprises a nucleotide sequence having at least 90% sequence identity to the nucleic acid sequence of SEQ ID NOs: 44. In some embodiments, the ampicillin resistance gene comprises a nucleotide sequence having at least 95% sequence identity to the nucleic acid sequence of SEQ ID NO: 44. In some embodiments, the ampicillin resistance gene comprises a nucleotide sequence having at least 97% sequence identity to the nucleic acid sequence of SEQ ID NO: 44. In some embodiments, the ampicillin resistance gene comprises a nucleotide sequence having at least 98% sequence identity to the nucleic acid sequence of SEQ ID NO: 44. In some embodiments, the ampicillin resistance gene comprises a nucleotide sequence having at least 99% sequence identity to the nucleic acid sequence of SEQ ID NO: 44. In some embodiments, the ampicillin resistance gene comprises a nucleotide sequence having the nucleic acid sequence of SEQ ID NO: 44.

In some embodiments, an ampicillin resistance gene comprises a nucleotide sequence having at least 70% (e.g., 75%, 80%, 90%, 95%, 97%, 98%, or 99%) sequence identity to the nucleic acid sequence of SEQ ID NO: 55. In some embodiments, the ampicillin resistance gene comprises a nucleotide sequence having at least 75% sequence identity to the nucleic acid sequence of SEQ ID NO: 55. In some embodiments, the ampicillin resistance gene comprises a nucleotide sequence having at least 80% sequence identity to the nucleic acid sequence of SEQ ID NO: 55. In some embodiments, the ampicillin resistance gene comprises a nucleotide sequence having at least 90% sequence identity to the nucleic acid sequence of SEQ ID NO: 55. In some embodiments, the ampicillin resistance gene comprises a nucleotide sequence having at least 95% sequence identity to the nucleic acid sequence of SEQ ID NO: 55. In some embodiments, the ampicillin resistance gene comprises a nucleotide sequence having at least 97% sequence identity to the nucleic acid sequence of SEQ ID NO: 55. In some embodiments, the ampicillin resistance gene comprises a nucleotide sequence having at least 98% sequence identity to the nucleic acid sequence of SEQ ID NO: 55. In some embodiments, the ampicillin resistance gene comprises a nucleotide sequence having at least 99% sequence identity to the nucleic acid sequence of SEQ ID NO: 55. In some embodiments, the ampicillin resistance gene comprises a nucleotide sequence having the nucleic acid sequence of SEQ ID NO: 55.

In some embodiments, a kanamycin resistance gene comprises a nucleotide sequence having at least 70% (e.g., 75%, 80%, 90%, 95%, 97%, 98%, or 99%) sequence identity to the nucleic acid sequence of SEQ ID NOs: 20 or 21. In some embodiments, the kanamycin resistance gene comprises a nucleotide sequence having at least 75% sequence identity to the nucleic acid sequence of SEQ ID NOs: 20 or 21. In some embodiments, the kanamycin resistance gene comprises a nucleotide sequence having at least 80% sequence identity to the nucleic acid sequence of SEQ ID NOs: 20 or 21. In some embodiments, the kanamycin resistance gene comprises a nucleotide sequence having at least 90% sequence identity to the nucleic acid sequence of SEQ ID NOs: 20 or 21. In some embodiments, the kanamycin resistance gene comprises a nucleotide sequence having at least 95% sequence identity to the nucleic acid sequence of SEQ ID NOs: 20 or 21. In some embodiments, the kanamycin resistance gene comprises a nucleotide sequence having at least 97% sequence identity to the nucleic acid sequence of SEQ ID NOs: 20 or 21. In some embodiments, the kanamycin resistance gene comprises a nucleotide sequence having at least 98% sequence identity to the nucleic acid sequence of SEQ ID NOs: 20 or 21. In some embodiments, the kanamycin resistance gene comprises a nucleotide sequence having at least 99% sequence identity to the nucleic acid sequence of SEQ ID NOs: 20 or 21. In some embodiments, the kanamycin resistance gene comprises a nucleotide sequence having the nucleic acid sequence of SEQ ID NOs: 20 or 21.

In some embodiments, the antibiotic resistance gene is operably linked to a promoter. In some embodiments, the promoter comprises a polynucleotide sequence as set forth in SEQ ID NO: 45, SEQ ID NO: 46, or SEQ ID NO: 47. In some embodiments, the promoter comprises a polynucleotide sequence as set forth in SEQ ID NO: 45. In some embodiments, the promoter comprises a polynucleotide sequence as set forth in SEQ ID NO: 46. In some embodiments, the promoter comprises a polynucleotide sequence as set forth in SEQ ID NO: 47. In some embodiments, the promoter comprises a polynucleotide sequence having at least 70% (e.g., 75%, 80%, 90%, 95%, 97%, 98%, or 99%) sequence identity to SEQ ID NO: 45, SEQ ID NO: 46, or SEQ ID NO: 47.

In some embodiments, the antibiotic resistance gene (e.g., any antibiotic resistance gene described herein, e.g., a kanamycin resistance gene) comprises reduced CpG dinucleotides and/or increased methylation of CpG dinucleotides as compared to a parental equivalent.

In some embodiments, methylation of CpG dinucleotides in the antibiotic resistance gene (e.g., any antibiotic resistance gene described herein, e.g., a kanamycin resistance gene) is increased by at least about 5%, 10%, 15%, 20%, 25%, 30%, 35%, 40%, 45%, 50%, 55%, 60%, 65%, 70%, 75%, 80%, 85%, 90%, 95%, or more than 95% as compared to a parental equivalent. In some embodiments, CpG dinucleotides are completely methylated.

In some embodiments, CpG dinucleotides in an antibiotic resistance gene (e.g., any antibiotic resistance gene described herein, e.g., a kanamycin resistance gene) are reduced by at least about 5%, 10%, 15%, 20%, 25%, 30%, 35%, 40%, 45%, 50%, 55%, 60%, 65%, 70%, 75%, 80%, 85%, 90%, 95%, or more than 99% as compared to a parental equivalent. In some embodiments, CpG dinucleotides in an antibiotic resistance gene are depleted.

In some embodiments, the rAAV vector further comprises an origin of replication. In some embodiments, the origin of replication is derived from plasmids such as pMB1, pBR322, ColE1, R6K, p15A, pSC101, ColE2, F1, pUC, pBluescript and/or combinations or a derivative thereof. In some embodiments, the origin of replication is pMB1 or a derivative thereof. In some embodiments, the origin of replication is pBR322 or a derivative thereof. In some embodiments, the origin of replication is ColE1 or a derivative thereof. In some embodiments, the origin of replication is R6K or a derivative thereof. In some embodiments, the origin of replication is p15A or a derivative thereof. In some embodiments, the origin of replication is pSC101 or a derivative thereof. In some embodiments, the origin of replication is ColE2 or a derivative thereof. In some embodiments, the origin of replication is F1 or a derivative thereof. In some embodiments, the origin of replication is pUC or a derivative thereof. In some embodiments, the origin of replication is pBluescript or a derivative thereof.

In some embodiments, the rAAV vector comprises a woodchuck hepatitis virus posttranscriptional regulatory element (WPRE) or a derivative thereof. In some embodiments, the WPRE is the mut6 variant of WPRE.

The rAAV vector described herein, in some embodiments, comprise a reporter sequence for co-expression, such as but not limited to lacZ, GFP (e.g., enhanced GFP (eGFP)), CFP, YFP, RFP, BFP, mCherry, mCardinal, Firefly luciferase (fLuc), Renilla luciferase, NanoLuc luciferase (nLuc), and tdTomato. In some embodiments, the rAAV vector comprises a selectable marker.

In some embodiments, the reporter sequence is mCardinal. In some embodiments, the mCardinal comprises a polynucleotide sequence having at least 70% (e.g., 75%, 80%, 90%, 95%, 97%, 98%, or 99%) sequence identity to SEQ ID NO: 56 or 57.

In some embodiments, the reporter sequence is eGFP. In some embodiments, the eGFP comprises a polynucleotide sequence having at least 70% (e.g., 75%, 80%, 90%, 95%, 97%, 98%, or 99%) sequence identity to SEQ ID NO: 58 or 59.

In some embodiments, the reporter sequence is fLuc. In some embodiments, the fLuc comprises a polynucleotide sequence having at least 70% (e.g., 75%, 80%, 90%, 95%, 97%, 98%, or 99%) sequence identity to SEQ ID NO: 66-68.

In some embodiments, the reporter sequence is nLuc. In some embodiments, the nLuc comprises a polynucleotide sequence having at least 70% (e.g., 75%, 80%, 90%, 95%, 97%, 98%, or 99%) sequence identity to SEQ ID NO: 69-70.

### Recombinant Viral Vectors

In some embodiments, a recombinant adeno-associated viral (rAAV) vector comprises any polynucleotide as described herein, or any plasmid as described herein. In some embodiments, the viral vector is derived from an anellovirus. In some embodiments, the viral vectors are derived from a parvovirus (e.g., AAV and bocavirus), retrovirus (e.g., Retroviridae family viral vector), adenovirus (e.g., Ad5, Ad26, Ad34, Ad35, and Ad48), anellovirus, arenavirus, coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus (e.g., poliovirus), and double stranded DNA viruses comprising adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses comprise Norwalk virus, togavirus, flavivirus, reoviruses, papovavirus, hepadnavirus, human papilloma virus, human foamy virus, and hepatitis virus, for example. Examples of retroviruses are avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus. Other examples are murine leukemia viruses, murine sarcoma viruses, murine mammary tumor virus, bovine leukemia virus, feline leukemia virus, feline sarcoma virus, avian leukemia virus, human T-cell leukemia virus, baboon endogenous virus, Gibbon ape leukemia virus, Pfizer monkey virus, simian immunodeficiency virus, simian sarcoma virus, Rous sarcoma virus and lentiviruses.

Various recombinant viral vectors are contemplated herein. In some embodiments, the recombinant viral vector is a recombinant herpesvirus is Herpes Simplex Virus (HSV), such as HSV type 1 (HSV-1), HSV-2, Varicella Zoster Virus (VZV), Epstein-Barr Virus (EBV), Cytomegalovirus (CMV), human herpes virus (HHV) 6 (HHV-6), HHV-7, or HHV-8. In some embodiments, the HSV is HSV-1. In some embodiments, the HSV is HSV-2. In some embodiments, the HSV is VZV. In some embodiments, the HSV is EBV. In some embodiments, the HSV is CMV. In some embodiments, the HSV is HHV-6. In some embodiments, the HSV is HHV-7. In some embodiments, the HSV is HHV-8.

In some embodiments, the recombinant viral vector is an AAV, such as AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16. In some embodiments, the recombinant AAV vector is an engineered AAV vector.

In some embodiments, a recombinant AAV is AAV1 or a derivative thereof. In some embodiments, a recombinant AAV is AAV2 or a derivative thereof. In some embodiments, a recombinant AAV is AAV3 or a derivative thereof. In some embodiments, a recombinant AAV is AAV4 or a derivative thereof. In some embodiments, a recombinant AAV is AAV5 or a derivative thereof. In some embodiments, a recombinant AAV is AAV6 or a derivative thereof. In some embodiments, a recombinant AAV is AAV7 or a derivative thereof. In some embodiments, a recombinant AAV is AAV8 or a derivative thereof. In some embodiments, a recombinant AAV is AAV9 or a derivative thereof. In some embodiments, a recombinant AAV is AAV10 or a derivative thereof. In some embodiments, a recombinant AAV is AAV11 or a derivative thereof. In some embodiments, a recombinant AAV is AAV12 or a derivative thereof. In some embodiments, a recombinant AAV is AAV13 or a derivative thereof. In some embodiments, a recombinant AAV is AAV14 or a derivative thereof. In some embodiments, a recombinant AAV is AAV15 or a derivative thereof. In some embodiments, a recombinant AAV is AAV16 or a derivative thereof. In some embodiments, a recombinant AAV is AAV-rh8 or a derivative thereof. In some embodiments, a recombinant AAV is AAV-rh10 or a derivative thereof. In some embodiments, a recombinant AAV is AAV-rh20 or a derivative thereof. In some embodiments, a recombinant AAV is AAV-rh39 or a derivative thereof. In some embodiments, a recombinant AAV is AAV-rh74 or a derivative thereof. In some embodiments, a recombinant AAV is AAV-rhM4-1 or a derivative thereof. In some embodiments, a recombinant AAV is AAV-hu37 or a derivative thereof. In some embodiments, a recombinant AAV is AAV-Anc80 or a derivative thereof. In some embodiments, a recombinant AAV is AAV-Anc80L65 or a derivative thereof. In some embodiments, a recombinant AAV is AAV-7m8 or a derivative thereof. In some embodiments, a recombinant AAV is AAV-PHP-B or a derivative thereof. In some embodiments, a recombinant AAV is AAV-PHP-EB or a derivative thereof. In some embodiments, a recombinant AAV is AAV-2.5 or a derivative thereof. In some embodiments, a recombinant AAV is AAV-2tYF or a derivative thereof. In some embodiments, a recombinant AAV is AAV-3B or a derivative thereof. In some embodiments, a recombinant AAV is AAV-LK03 or a derivative thereof. In some embodiments, a recombinant AAV is AAV-HSC1 or a derivative thereof. In some embodiments, a recombinant AAV is AAV-HSC2 or a derivative thereof. In some embodiments, a recombinant AAV is AAV-HSC3 or a derivative thereof. In some embodiments, a recombinant AAV is AAV-HSC4 or a derivative thereof. In some embodiments, a recombinant AAV is AAV-HSC5 or a derivative thereof. In some embodiments, a recombinant AAV is AAV-HSC6 or a derivative thereof. In some embodiments, a recombinant AAV is AAV-HSC7 or a derivative thereof. In some embodiments, a recombinant AAV is AAV-HSC8 or a derivative thereof. In some embodiments, a recombinant AAV is AAV-HSC9 or a derivative thereof. In some embodiments, a recombinant AAV is AAV-HSC10 or a derivative thereof. In some embodiments, a recombinant AAV is AAV-HSC11 or a derivative thereof. In some embodiments, a recombinant AAV is AAV-HSC12 or a derivative thereof. In some embodiments, a recombinant AAV is AAV-HSC13 or a derivative thereof. In some embodiments, a recombinant AAV is AAV-HSC14 or a derivative thereof. In some embodiments, a recombinant AAV is AAV-HSC15 or a derivative thereof. In some embodiments, a recombinant AAV is AAV-TT or a derivative thereof. In some embodiments, a recombinant AAV is AAV-DJ/8 or a derivative thereof. In some embodiments, a recombinant AAV is AAV-Myo or a derivative thereof. In some embodiments, a recombinant AAV is AAV-NP40 or a derivative thereof. In some embodiments, a recombinant AAV is AAV-NP59 or a derivative thereof. In some embodiments, a recombinant AAV is AAV-NP22 or a derivative thereof. In some embodiments, a recombinant AAV is AAV-NP66 or a derivative thereof. In some embodiments, a recombinant AAV is AAV-HSC16 or a derivative thereof.

### Recombinant AAV Vectors

Recombinant adeno-associated viral vectors (rAAV) can be used for incorporation of genes (e.g., polynucleotides encoding a payload of interest) to facilitate their introduction into a cell, such as a target cell. The present disclosure provides rAAV vectors comprising any nucleic acid described herein. Among other things, described herein rAAV vectors comprising nucleic acids encoding one or more payloads of interest. In some embodiments, one or more payloads of interest encode one or more interferons, as described herein. In some embodiments, the rAAV vector comprises a nucleic acid that encodes a single interferon polypeptide. In some embodiments, the rAAV vector comprises a bicistronic or tricistronic nucleic acid that encodes multiple interferon polypeptides.

In some embodiments, the polynucleotides described herein encode one or more payloads of interest (e.g., therapeutic polypeptides, such as cytokines) to be delivered to a cell or tissue, as well as regulatory elements controlling expression of the one or more payloads of interest. In some embodiments, the polynucleotides described herein encode one or more interferons to be delivered to a cell or tissue, as well as regulatory elements controlling expression of the one or more interferons. Regulatory elements include, but are not limited to, promoters, enhancers, polyadenylation sequences, introns, synthetic introns, mRNA stability sequences (e.g. Woodchuck Hepatitis Virus Posttranscriptional Regulatory Element; WPRE), sequences that allow for internal ribosome entry sites (IRES) of bicistronic mRNA, sequences necessary for episome maintenance (e.g., ITRs), sequences that avoid or inhibit viral recognition by Toll-like or RIG-like receptors (e.g. TLR-7, -8, -9, MDA-5, RIG-1 and/or DAI), and/or sequences necessary for transduction into cells.

In some embodiments, the rAAV vectors described herein comprise a CAG promoter operably linked to one or more polynucleotides encoding one or more interferon polypeptides. In some embodiments, the CAG promoter comprises a first segment comprising a cytomegalovirus (CMV) enhancer sequence, a second segment comprising a chicken beta-actin (CBA) gene promoter element, a third segment comprising a spacer sequence, and a fourth segment comprising a rabbit beta-globin splice acceptor. In some embodiments, the order of the segments 5-prime to 3-prime is first, second, third, and fourth.

In some embodiments, the CAG promoter comprises the nucleic acid sequence of any one of SEQ ID NOs: 22-23. In some embodiments, the CAG promoter comprises a nucleic acid sequence having at least 70% (e.g., 75%, 80%, 90%, 95%, 97%, 98%, or 99%) sequence identity to the nucleic acid sequence of any one of SEQ ID NOs: 22-23. In some embodiments, the CAG promoter comprises a nucleic acid sequence having at least 75% sequence identity to the nucleic acid sequence of any one of SEQ ID NOs: 22-23. In some embodiments, the CAG promoter comprises a nucleic acid sequence having at least 80% sequence identity to the nucleic acid sequence of any one of SEQ ID NOs: 22-23. In some embodiments, the CAG promoter comprises a nucleic acid sequence having at least 90% sequence identity to the nucleic acid sequence of any one of SEQ ID NOs: 22-23. In some embodiments, the CAG promoter comprises a nucleic acid sequence having at least 95% sequence identity to the nucleic acid sequence of any one of SEQ ID NOs: 22-23. In some embodiments, the CAG promoter comprises a nucleic acid sequence having at least 97% sequence identity to the nucleic acid sequence of any one of SEQ ID NOs: 22-23. In some embodiments, the CAG promoter comprises a nucleic acid sequence having at least 98% sequence identity to the nucleic acid sequence of any one of SEQ ID NOs: 22-23. In some embodiments, the CAG promoter comprises a nucleic acid sequence having at least 99% sequence identity to the nucleic acid sequence of any one of SEQ ID NOs: 22-23. In some embodiments, the CAG promoter comprises contiguous nucleotides of about 20, about 25, about 40, about 50, about 75, about 100, about 125, about 150, about 175, about 180, about 200, about 220, about 240, about 260, about 280, about 300, about 320, about 340, about 360, about 380, about 400, or about 240 nucleotides of any one of SEQ ID NOs: 22-23. In some embodiments, the CAG promoter comprises at least 70% (e.g., 75%, 80%, 90%, 95%, 97%, 98%, or 99%) sequence identity to contiguous nucleotides of about 20, about 25, about 40, about 50, about 75, about 100, about 125, about 150, about 175, about 180, about 200, about 220, about 240, about 260, about 280, about 300, about 320, about 340, about 360, about 380, about 400, or about 240 nucleotides of any one of SEQ ID NOs: 22-23. In some embodiments, the CAG promoter comprises the nucleic acid sequence of any one of SEQ ID NOs: 48-50 and 64-65. In some embodiments, the CAG promoter comprises a nucleic acid sequence having at least 70% (e.g., 75%, 80%, 90%, 95%, 97%, 98%, or 99%) sequence identity to the nucleic acid sequence of any one of SEQ ID NOs: 48-50 and 64-65. In some embodiments, the CAG promoter comprises a nucleic acid sequence having at least 75% sequence identity to the nucleic acid sequence of any one of SEQ ID NOs: 48-50 and 64-65. In some embodiments, the CAG promoter comprises a nucleic acid sequence having at least 80% sequence identity to the nucleic acid sequence of any one of SEQ ID NOs: 48-50 and 64-65. In some embodiments, the CAG promoter comprises a nucleic acid sequence having at least 90% sequence identity to the nucleic acid sequence of any one of SEQ ID NOs: 48-50 and 64-65. In some embodiments, the CAG promoter comprises a nucleic acid sequence having at least 95% sequence identity to the nucleic acid sequence of any one of SEQ ID NOs: 48-50 and 64-65. In some embodiments, the CAG promoter comprises a nucleic acid sequence having at least 97% sequence identity to the nucleic acid sequence of any one of SEQ ID NOs: 48-50 and 64-65. In some embodiments, the CAG promoter comprises a nucleic acid sequence having at least 98% sequence identity to the nucleic acid sequence of any one of SEQ ID NOs: 48-50 and 64-65. In some embodiments, the CAG promoter comprises a nucleic acid sequence having at least 99% sequence identity to the nucleic acid sequence of any one of SEQ ID NOs: 48-50 and 64-65. In some embodiments, the CAG promoter comprises contiguous nucleotides of about 20, about 25, about 40, about 50, about 75, about 100, about 125, about 150, about 175, about 180, about 200, about 220, about 240, about 260, about 280, about 300, about 320, about 340, about 360, about 380, about 400, or about 240 nucleotides of any one of SEQ ID NOs: 48-50 and 64-65. In some embodiments, the CAG promoter comprises at least 70% (e.g., 75%, 80%, 90%, 95%, 97%, 98%, or 99%) sequence identity to contiguous nucleotides of about 20, about 25, about 40, about 50, about 75, about 100, about 125, about 150, about 175, about 180, about 200, about 220, about 240, about 260, about 280, about 300, about 320, about 340, about 360, about 380, about 400, or about 240 nucleotides of any one of SEQ ID NOs: 48-50 and 64-65.

In some embodiments, the CMV enhancer is derived from human CMV. In some embodiments, the CMV enhancer comprises various repeated sequence elements. In some embodiments, the CMV enhancer comprises the nucleic acid sequence of any one of SEQ ID NOs: 25-27. In some embodiments, the CMV enhancer comprises a nucleic acid sequence having at least 70% (e.g., 75%, 80%, 90%, 95%, 97%, 98%, or 99%) sequence identity to the nucleic acid sequence of any one of SEQ ID NOs: 25-27. In some embodiments, the CMV enhancer comprises a nucleic acid sequence having at least 75% sequence identity to the nucleic acid sequence of any one of SEQ ID NOs: 25-27. In some embodiments, the CMV enhancer comprises a nucleic acid sequence having at least 80% sequence identity to the nucleic acid sequence of any one of SEQ ID NOs: 25-27. In some embodiments, the CMV enhancer comprises a nucleic acid sequence having at least 90% sequence identity to the nucleic acid sequence of any one of SEQ ID NOs: 25-27. In some embodiments, the CMV enhancer comprises a nucleic acid sequence having at least 95% sequence identity to the nucleic acid sequence of any one of SEQ ID NOs: 25-27. In some embodiments, the CMV enhancer comprises a nucleic acid sequence having at least 97% sequence identity to the nucleic acid sequence of any one of SEQ ID NOs: 25-27. In some embodiments, the CMV enhancer comprises a nucleic acid sequence having at least 98% sequence identity to the nucleic acid sequence of any one of SEQ ID NOs: 25-27. In some embodiments, the CMV enhancer comprises a nucleic acid sequence having at least 99% sequence identity to the nucleic acid sequence of any one of SEQ ID NOs: 25-27. In some embodiments, the CMV enhancer comprises a nucleic acid sequence having the nucleic acid sequence of any one of SEQ ID NOs: 25-27. In some embodiments, the CMV enhancer comprises contiguous nucleotides of about 20, about 25, about 40, about 50, about 75, about 100, about 125, about 150, about 175, about 180, about 200, about 220, about 240, about 260, about 280, about 300, about 320, about 340, about 360, about 380, about 400, or about 240 nucleotides of any one of SEQ ID NOs: 25-27. In some embodiments, the CMV enhancer comprises at least 70% (e.g., 75%, 80%, 90%, 95%, 97%, 98%, or 99%) sequence identity to contiguous nucleotides of about 20, about 25, about 40, about 50, about 75, about 100, about 125, about 150, about 175, about 180, about 200, about 220, about 240, about 260, about 280, about 300, about 320, about 340, about 360, about 380, about 400, or about 240 nucleotides of any one of SEQ ID NOs: 25-27.

In some embodiments, the CAG promoter comprises a chicken beta-actin (CBA) gene promoter element. In some embodiments, the CBA gene promoter element comprises a CBA gene promoter sequence, a CBA gene first exon, and a CBA gene first intron. In some embodiments, the CBA promoter comprises the nucleic acid sequence of SEQ ID NO: 28. In some embodiments, the CBA promoter comprises a nucleic acid sequence having at least 70% (e.g., 75%, 80%, 90%, 95%, 97%, 98%, or 99%) sequence identity to the nucleic acid sequence of SEQ ID NO: 28. In some embodiments, the CBA promoter comprises a nucleic acid sequence having at least 75% sequence identity to the nucleic acid sequence of SEQ ID NO: 28. In some embodiments, the CBA promoter comprises a nucleic acid sequence having at least 80% sequence identity to the nucleic acid sequence of SEQ ID NO: 28. In some embodiments, the CBA promoter comprises a nucleic acid sequence having at least 90% sequence identity to the nucleic acid sequence of SEQ ID NO: 28. In some embodiments, the CBA promoter comprises a nucleic acid sequence having at least 95% sequence identity to the nucleic acid sequence of SEQ ID NO: 28. In some embodiments, the CBA promoter comprises a nucleic acid sequence having at least 97% sequence identity to the nucleic acid sequence of SEQ ID NO: 28. In some embodiments, the CBA promoter comprises a nucleic acid sequence having at least 98% sequence identity to the nucleic acid sequence of SEQ ID NO: 28. In some embodiments, the CBA promoter comprises a nucleic acid sequence having at least 99% sequence identity to the nucleic acid sequence of SEQ ID NO: 28. In some embodiments, the CBA promoter comprises a nucleic acid sequence having the nucleic acid sequence of SEQ ID NO: 28. In some embodiments, the CBA promoter comprises contiguous nucleotides of about 20, about 25, about 40, about 50, about 75, about 100, about 125, about 150, about 175, about 180, about 200, about 220, about 240, about 260, about 280, about 300, about 320, about 340, about 360, about 380, about 400, or about 240 nucleotides of SEQ ID NO: 28. In some embodiments, the CBA promoter comprises at least 70% (e.g., 75%, 80%, 90%, 95%, 97%, 98%, or 99%) sequence identity to contiguous nucleotides of about 20, about 25, about 40, about 50, about 75, about 100, about 125, about 150, about 175, about 180, about 200, about 220, about 240, about 260, about 280, about 300, about 320, about 340, about 360, about 380, about 400, or about 240 nucleotides of SEQ ID NO: 28. In some embodiments, the CBA promoter comprises a nucleic acid sequence having at least 70% (e.g., 75%, 80%, 90%, 95%, 97%, 98%, or 99%) sequence identity to the nucleic acid sequence of SEQ ID NO: 52. In some embodiments, the CBA promoter comprises a nucleic acid sequence having at least 75% sequence identity to the nucleic acid sequence of SEQ ID NO: 52. In some embodiments, the CBA promoter comprises a nucleic acid sequence having at least 80% sequence identity to the nucleic acid sequence of SEQ ID NO: 52. In some embodiments, the CBA promoter comprises a nucleic acid sequence having at least 90% sequence identity to the nucleic acid sequence of SEQ ID NO: 52. In some embodiments, the CBA promoter comprises a nucleic acid sequence having at least 95% sequence identity to the nucleic acid sequence of SEQ ID NO: 52. In some embodiments, the CBA promoter comprises a nucleic acid sequence having at least 97% sequence identity to the nucleic acid sequence of SEQ ID NO: 52. In some embodiments, the CBA promoter comprises a nucleic acid sequence having at least 98% sequence identity to the nucleic acid sequence of SEQ ID NO: 52. In some embodiments, the CBA promoter comprises a nucleic acid sequence having at least 99% sequence identity to the nucleic acid sequence of SEQ ID NO: 52. In some embodiments, the CBA promoter comprises a nucleic acid sequence having the nucleic acid sequence of SEQ ID NO: 52. In some embodiments, the CBA promoter comprises contiguous nucleotides of about 20, about 25, about 40, about 50, about 75, about 100, about 125, about 150, about 175, about 180, about 200, about 220, about 240, about 260, about 520, about 300, about 320, about 340, about 360, about 380, about 400, or about 240 nucleotides of SEQ ID NO: 52. In some embodiments, the CBA promoter comprises at least 70% (e.g., 75%, 80%, 90%, 95%, 97%, 98%, or 99%) sequence identity to contiguous nucleotides of about 20, about 25, about 40, about 50, about 75, about 100, about 125, about 150, about 175, about 180, about 200, about 220, about 240, about 260, about 520, about 300, about 320, about 340, about 360, about 380, about 400, or about 240 nucleotides of SEQ ID NO: 52.

In some embodiments, the CAG promoter comprises a spacer sequence immediately 3′ to the CBA promoter element. In some embodiments, the spacer contains an intron element. In some embodiment the spacer contains an exon element. In some embodiments, the spacer sequence is about 5 to about 10, 12, 15, 20, 25, 30, 35, 40, 45, 50, 60, 70, 80, 90, 100, 120, 140, 160, 180, 200, or 250 nucleotides in length. In some embodiments, the spacer sequence is about 10 to about 12, 15, 20, 25, 30, 35, 40, 45, 50, 60, 70, 80, 90, 100, 120, 140, 160, 180, 200, or 250 nucleotides in length. In some embodiments, the spacer sequence is about 20 to about 25, 30, 35, 40, 45, 50, 60, 70, 80, 90, 100, 120, 140, 160, 180, 200, or 250 nucleotides in length. In some embodiments, the spacer sequence is about 50 to about 60, 70, 80, 90, 100, 120, 140, 160, 180, 200, or 250 nucleotides in length. In some embodiments, the spacer sequence is about 80 to about 100, 120, 140, 160, 180, 200, or 250 nucleotides in length. In some embodiments, the spacer sequence is at least 10 nucleotides in length. In some embodiments, the spacer sequence is at least 12 nucleotides in length. In some embodiments, the spacer sequence is at least 20 nucleotides in length. In some embodiments, the spacer sequence is at least 50 nucleotides in length. In some embodiments, the spacer sequence is at least 100 nucleotides in length. In some embodiments, the spacer sequence is at least 250 nucleotides in length. In some embodiments, the spacer sequence is 5 to 20 nucleotides in length. In some embodiments the spacer is 10 to 20 nucleotides in length. In some embodiments, the spacer sequence is 10 to 20 nucleotides in length. In some embodiments, the spacer sequence is 10 to 15 nucleotides in length. In some embodiments the spacer sequence is 250 to 350 nucleotides in length. In some embodiments, the spacer sequence comprises the nucleic acid sequence of SEQ ID NO: 29. In some embodiments, the spacer sequence comprises a nucleic acid sequence having at least 70% (e.g., 75%, 80%, 90%, 95%, 97%, 98%, or 99%) sequence identity to the nucleic acid sequence of SEQ ID NO: 29. In some embodiments, the spacer sequence comprises a nucleic acid sequence having at least 75% sequence identity to the nucleic acid sequence of SEQ ID NO: 29. In some embodiments, the spacer sequence comprises a nucleic acid sequence having at least 80% sequence identity to the nucleic acid sequence of SEQ ID NO: 29. In some embodiments, the spacer sequence comprises a nucleic acid sequence having at least 90% sequence identity to the nucleic acid sequence of SEQ ID NO: 29. In some embodiments, the spacer sequence comprises a nucleic acid sequence having at least 95% sequence identity to the nucleic acid sequence of SEQ ID NO: 29. In some embodiments, the spacer sequence comprises a nucleic acid sequence having at least 97% sequence identity to the nucleic acid sequence of SEQ ID NO: 29. In some embodiments, the spacer sequence comprises a nucleic acid sequence having at least 98% sequence identity to the nucleic acid sequence of SEQ ID NO: 29. In some embodiments, the spacer sequence comprises a nucleic acid sequence having at least 99% sequence identity to the nucleic acid sequence of SEQ ID NO: 29. In some embodiments, the spacer sequence comprises a nucleic acid sequence having the nucleic acid sequence of SEQ ID NO: 29. In some embodiments, the spacer sequence is 350 to 1000 nucleotides in length. In some embodiments, the spacer sequence is 500 to 1000 nucleotides in length. In some embodiments, the spacer sequence is 600 to 1000 nucleotides in length. In some embodiments, the spacer sequence is 700 to 1000 nucleotides in length. In some embodiments, the spacer sequence is 800 to 1000 nucleotides in length. In some embodiments, the spacer sequence is 900 to 1000 nucleotides in length.

In some embodiments, the CAG promoter comprises a rabbit beta-globin splice acceptor. In some embodiments, the rabbit beta-globin splice acceptor comprises the nucleic acid sequence of SEQ ID NO: 30. In some embodiments, the rabbit beta-globin splice acceptor comprises a nucleic acid sequence having at least 70% (e.g., 75%, 80%, 90%, 95%, 97%, 98%, or 99%) sequence identity to the nucleic acid sequence of SEQ ID NO: 30. In some embodiments, the rabbit beta-globin splice acceptor comprises a nucleic acid sequence having at least 75% sequence identity to the nucleic acid sequence of SEQ ID NO: 30. In some embodiments, the rabbit beta-globin splice acceptor comprises a nucleic acid sequence having at least 80% sequence identity to the nucleic acid sequence of SEQ ID NO: 30. In some embodiments, the rabbit beta-globin splice acceptor comprises a nucleic acid sequence having at least 90% sequence identity to the nucleic acid sequence of SEQ ID NO: 30. In some embodiments, the rabbit beta-globin splice acceptor comprises a nucleic acid sequence having at least 95% sequence identity to the nucleic acid sequence of SEQ ID NO: 30. In some embodiments, the rabbit beta-globin splice acceptor comprises a nucleic acid sequence having at least 97% sequence identity to the nucleic acid sequence of SEQ ID NO: 30. In some embodiments, the rabbit beta-globin splice acceptor comprises a nucleic acid sequence having at least 98% sequence identity to the nucleic acid sequence of SEQ ID NO: 30. In some embodiments, the rabbit beta-globin splice acceptor comprises a nucleic acid sequence having at least 99% sequence identity to the nucleic acid sequence of SEQ ID NO: 30. In some embodiments, the rabbit beta-globin splice acceptor comprises a nucleic acid sequence having the nucleic acid sequence of SEQ ID NO: 30. In some embodiments, the CAG promoter comprises a rabbit beta-globin splice acceptor. In some embodiments, the rabbit beta-globin splice acceptor comprises the nucleic acid sequence of SEQ ID NO: 53. In some embodiments, the rabbit beta-globin splice acceptor comprises a nucleic acid sequence having at least 70% (e.g., 75%, 80%, 90%, 95%, 97%, 98%, or 99%) sequence identity to the nucleic acid sequence of SEQ ID NO: 53. In some embodiments, the rabbit beta-globin splice acceptor comprises a nucleic acid sequence having at least 75% sequence identity to the nucleic acid sequence of SEQ ID NO: 53. In some embodiments, the rabbit beta-globin splice acceptor comprises a nucleic acid sequence having at least 80% sequence identity to the nucleic acid sequence of SEQ ID NO: 53. In some embodiments, the rabbit beta-globin splice acceptor comprises a nucleic acid sequence having at least 90% sequence identity to the nucleic acid sequence of SEQ ID NO: 53. In some embodiments, the rabbit beta-globin splice acceptor comprises a nucleic acid sequence having at least 95% sequence identity to the nucleic acid sequence of SEQ ID NO: 53. In some embodiments, the rabbit beta-globin splice acceptor comprises a nucleic acid sequence having at least 97% sequence identity to the nucleic acid sequence of SEQ ID NO: 53. In some embodiments, the rabbit beta-globin splice acceptor comprises a nucleic acid sequence having at least 98% sequence identity to the nucleic acid sequence of SEQ ID NO: 53. In some embodiments, the rabbit beta-globin splice acceptor comprises a nucleic acid sequence having at least 99% sequence identity to the nucleic acid sequence of SEQ ID NO: 53. In some embodiments, the rabbit beta-globin splice acceptor comprises a nucleic acid sequence having the nucleic acid sequence of SEQ ID NO: 53.

Useful rAAV vectors comprise those having one or more of the naturally occurring AAV genes deleted in whole or in part, but which retain functional flanking ITR sequences. In some embodiments, the AAV ITRs are of any serotype suitable for a particular application. In some embodiments, the nucleic acid does not comprise ITRs. In some embodiments, the AAV ITRs are of any suitable AAV serotype, comprising any now known or later discovered serotypes or any engineered, evolved, selected, or chimeric capsid serotype. In some embodiments the ITRs are of a different length or configuration.

The capsid proteins of AAV compose the exterior, non-nucleic acid portion of the virion and are encoded by the AAV Cap gene. In some embodiments, the Cap gene encodes VP1, VP2, VP3, MAAP, AAP, or combinations thereof.

In some embodiments, provided herein are Rep, Cap, or other polynucleotides required for producing the rAAV of the disclosure. In some embodiments, the Rep, Cap, or other polynucleotides are delivered to the packaging host cell using any appropriate genetic element (e.g., vector). In some embodiments, a single nucleic acid encoding all three capsid proteins (e.g., VP1, VP2 and VP3) is delivered into the packaging host cell in a single vector. In some embodiments, nucleic acids encoding the capsid proteins are delivered into the packaging host cell by two vectors; a first vector comprising a first nucleic acid encoding two capsid proteins (e.g., VP1 and VP2) and a second vector comprising a second nucleic acid encoding a single capsid protein (e.g., VP3). In some embodiments, three vectors, each comprising a nucleic acid encoding a different capsid protein, are delivered to the packaging host cell.

In some embodiments, a single nucleic acid encoding multiple replication proteins (e.g., Rep78, Rep68, Rep52, and Rep40) is delivered into the packaging host cell in a single vector. In some embodiments, nucleic acids encoding the replication proteins are delivered into the packaging host cell by two vectors; a first vector comprising a first nucleic acid encoding one to three replication proteins and a second vector comprising a second nucleic acid encoding one to three replication proteins. In some embodiments, four vectors, each comprising a nucleic acid encoding a different replication protein, are delivered to the packaging host cell.

In some embodiments, a single nucleic acid encoding multiple adenovirus helper proteins (e.g., E1A, EIB, E4, E2A, and VA RNA) is delivered into the packaging host cell in a single vector. In some embodiments, nucleic acids encoding the adenovirus helper proteins are delivered into the packaging host cell by two vectors. In some embodiments, more than two vectors, each comprising a nucleic acid encoding a different capsid protein, are delivered to the packaging host cell. The selected genetic elements are delivered by any suitable method, comprising those described herein. The methods used to construct any embodiment of this disclosure comprise genetic engineering, recombinant engineering, and synthetic techniques.

In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16.

In some embodiments, a recombinant AAV is AAV1 or a derivative thereof. In some embodiments, a recombinant AAV is AAV2 or a derivative thereof. In some embodiments, a recombinant AAV is AAV3 or a derivative thereof. In some embodiments, a recombinant AAV is AAV4 or a derivative thereof. In some embodiments, a recombinant AAV is AAV5 or a derivative thereof. In some embodiments, a recombinant AAV is AAV6 or a derivative thereof. In some embodiments, a recombinant AAV is AAV7 or a derivative thereof. In some embodiments, a recombinant AAV is AAV8 or a derivative thereof. In some embodiments, a recombinant AAV is AAV9 or a derivative thereof. In some embodiments, a recombinant AAV is AAV10 or a derivative thereof. In some embodiments, a recombinant AAV is AAV11 or a derivative thereof. In some embodiments, a recombinant AAV is AAV12 or a derivative thereof. In some embodiments, a recombinant AAV is AAV13 or a derivative thereof. In some embodiments, a recombinant AAV is AAV14 or a derivative thereof. In some embodiments, a recombinant AAV is AAV15 or a derivative thereof. In some embodiments, a recombinant AAV is AAV16 or a derivative thereof. In some embodiments, a recombinant AAV is AAV-rh8 or a derivative thereof. In some embodiments, a recombinant AAV is AAV-rh10 or a derivative thereof. In some embodiments, a recombinant AAV is AAV-rh20 or a derivative thereof. In some embodiments, a recombinant AAV is AAV-rh39 or a derivative thereof. In some embodiments, a recombinant AAV is AAV-rh74 or a derivative thereof. In some embodiments, a recombinant AAV is AAV-rhM4-1 or a derivative thereof. In some embodiments, a recombinant AAV is AAV-hu37 or a derivative thereof. In some embodiments, a recombinant AAV is AAV-Anc80 or a derivative thereof. In some embodiments, a recombinant AAV is AAV-Anc80L65 or a derivative thereof. In some embodiments, a recombinant AAV is AAV-7m8 or a derivative thereof. In some embodiments, a recombinant AAV is AAV-PHP-B or a derivative thereof. In some embodiments, a recombinant AAV is AAV-PHP-EB or a derivative thereof. In some embodiments, a recombinant AAV is AAV-2.5 or a derivative thereof. In some embodiments, a recombinant AAV is AAV-2tYF or a derivative thereof. In some embodiments, a recombinant AAV is AAV-3B or a derivative thereof. In some embodiments, a recombinant AAV is AAV-LK03 or a derivative thereof. In some embodiments, a recombinant AAV is AAV-HSC1 or a derivative thereof. In some embodiments, a recombinant AAV is AAV-HSC2 or a derivative thereof. In some embodiments, a recombinant AAV is AAV-HSC3 or a derivative thereof. In some embodiments, a recombinant AAV is AAV-HSC4 or a derivative thereof. In some embodiments, a recombinant AAV is AAV-HSC5 or a derivative thereof. In some embodiments, a recombinant AAV is AAV-HSC6 or a derivative thereof. In some embodiments, a recombinant AAV is AAV-HSC7 or a derivative thereof. In some embodiments, a recombinant AAV is AAV-HSC8 or a derivative thereof. In some embodiments, a recombinant AAV is AAV-HSC9 or a derivative thereof. In some embodiments, a recombinant AAV is AAV-HSC10 or a derivative thereof. In some embodiments, a recombinant AAV is AAV-HSC11 or a derivative thereof. In some embodiments, a recombinant AAV is AAV-HSC12 or a derivative thereof. In some embodiments, a recombinant AAV is AAV-HSC13 or a derivative thereof. In some embodiments, a recombinant AAV is AAV-HSC14 or a derivative thereof. In some embodiments, a recombinant AAV is AAV-HSC15 or a derivative thereof. In some embodiments, a recombinant AAV is AAV-TT or a derivative thereof. In some embodiments, a recombinant AAV is AAV-DJ/8 or a derivative thereof. In some embodiments, a recombinant AAV is AAV-Myo or a derivative thereof. In some embodiments, a recombinant AAV is AAV-NP40 or a derivative thereof. In some embodiments, a recombinant AAV is AAV-NP59 or a derivative thereof. In some embodiments, a recombinant AAV is AAV-NP22 or a derivative thereof. In some embodiments, a recombinant AAV is AAV-NP66 or a derivative thereof. In some embodiments, a recombinant AAV is AAV-HSC16 or a derivative thereof.

In some embodiments, the rAAV vector is a pseudotyped rAAV vector. Pseudotyped vectors comprise AAV genome vectors of a given serotype pseudotyped with Cap gene products derived from a serotype other than the given serotype (e.g., AAV1, AAV3, AAV4, AAV5, AAV6, AAV7, AAV8, or AAV9, among others). For example, a representative pseudotyped vector is an AAV serotype 2 genome vector pseudotyped with Cap gene products derived from AAV serotype 9.

In some embodiments, the AAV have mutations within the virion capsid that are used to transduce particular cell types more effectively than non-mutated capsid virions. In some embodiments, suitable AAV mutants have ligand insertions for the facilitation of targeting AAV to specific cell types. In some embodiments, the construction and characterization of AAV capsid mutants comprising insertion mutants, alanine screening mutants, and epitope tag mutants are used.

In some embodiments, artificial AAV capsids are used. Such an artificial capsid is generated by any suitable technique using a selected AAV sequence (e.g., a fragment of a VP1 capsid protein) in combination with heterologous sequences which are obtained from a different selected AAV serotype, non-contiguous portions of the same AAV serotype, from a non-AAV viral source, or from a non-viral source. An artificial AAV serotype are, without limitation, a pseudotyped AAV, a chimeric AAV capsid, a recombinant AAV capsid, an engineered AAV capsid, or a “humanized” AAV capsid.

Other rAAV virions that are used in compositions and methods of the disclosure include, but are not limited to, those capsid hybrids that are generated by molecular breeding of viruses as well as by exon shuffling, directed evolution, rational design, error-prone PCR, generated through computational algorithms, or generated through use of artificial intelligence or machine learning.

In some embodiments, the capsid is modified to improve therapy. In some embodiments, the capsid is modified for minimized immunogenicity and/or immune cloaking, better stability and/or particle durability, efficient degradation, and/or accurate delivery of the heterologous coding sequence or a functional fragment or variant thereof to the nucleus. In some embodiments, the modification or mutation is an amino acid deletion, insertion, substitution, or any combination thereof in a capsid polypeptide. In some embodiments, the capsid polypeptide comprises 1, 2, 3, 4, 5, up to 10, or more amino acid substitutions and/or deletions and/or insertions. In some embodiments, one or more amino acid substitutions are introduced into one or more of VP1, VP2 or VP3. In one embodiment, a modified capsid polypeptide comprises 1, 2, 3, 4, 5, 6, 7, 8, 9, 10, 11, 12, 13, 14, or 15 conservative or non-conservative substitutions relative to the wild-type polypeptide.

In another embodiment, the modified capsid polypeptide of the disclosure comprises modified sequences, wherein such modifications can comprise both conservative and non-conservative substitutions, deletions, and/or additions, and typically comprise peptides that share at least 40%, at least 45%, at least 50%, at least 55%, at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the corresponding wild-type capsid protein.

Methods for Generating rAAV Vectors

Described herein, are methods for generating rAAV vectors (e.g., rAAV vectors comprising a polynucleotide as described herein), comprising: contacting cells in vitro with the polynucleotides described herein and a transfer plasmid, Rep/Cap, a helper plasmid, or combinations thereof to generate the recombinant adeno-associated viral (AAV) vectors. In some embodiments, the cells are infected with the rAAV vectors. In some embodiments, the cells are transduced with the rAAV vectors. In some embodiments, the cells are engineered stable cell lines.

In some embodiments, the cell is a eukaryotic cell (e.g., a plant cell, an animal cell, a protist cell, or a fungi cell), a mammalian cell (a Chinese hamster ovary (CHO) cell, baby hamster kidney (BHK), human embryonic kidney (HEK-293), mouse myeloma (NSO), Vero, or human retinal cells), an immortalized cell (e.g., a HeLa cell, a COS cell, a HEK-293T cell, a MDCK cell, a 3T3 cell, a PC12 cell, a Huh7 cell, a HepG2 cell, a K562 cell, a N2a cell, or a SY5Y cell), an insect cell (e.g., a Spodoptera frugiperda cell, a Trichoplusia ni cell, a Drosophila melanogaster cell, a S2 cell, or a Heliothis virescens cell), a yeast cell (e.g., a Saccharomyces cerevisiae cell, a Cryptococcus cell, or a Candida cell), a plant cell (e.g., a parenchyma cell, a collenchyma cell, or a sclerenchyma cell), a fungal cell (e.g., a Saccharomyces cerevisiae cell, a Cryptococcus cell, or a Candida cell), or a prokaryotic cell (e.g., a E. coli cell, a Streptococcus bacterium cell, a Streptomyces soil bacteria cell, or an archaea cell). In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell.

In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, a primary cell, or derivative thereof. In some embodiments, the cell is a A549 cell or a derivative thereof. In some embodiments, the cell is a HEK-293 cell or a derivative thereof. In some embodiments, the cell is a HEK-293T cell or a derivative thereof. In some embodiments, the cell is a BHK cell or a derivative thereof. In some embodiments, the cell is a CHO cell or a derivative thereof. In some embodiments, the cell is a HeLa cell or a derivative thereof. In some embodiments, the cell is a MRC5 cell or a derivative thereof. In some embodiments, the cell is a Sf9 cell or a derivative thereof. In some embodiments, the cell is a Cos-1 cell or a derivative thereof. In some embodiments, the cell is a Cos-7 cell or a derivative thereof. In some embodiments, the cell is a Vero cell or a derivative thereof. In some embodiments, the cell is a BSC 1 cell or a derivative thereof. In some embodiments, the cell is a BSC 40 cell or a derivative thereof. In some embodiments, the cell is a BMT 10 cell or a derivative thereof. In some embodiments, the cell is a WI38 cell or a derivative thereof. In some embodiments, the cell is a HeLa cell or a derivative thereof. In some embodiments, the cell is a Saos cell or a derivative thereof. In some embodiments, the cell is a C2C12 cell or a derivative thereof. In some embodiments, the cell is a L cell or a derivative thereof. In some embodiments, the cell is a HT1080 cell or a derivative thereof. In some embodiments, the cell is a HepG2 cell or a derivative thereof. In some embodiments, the cell is a Huh7 cell or a derivative thereof. In some embodiments, the cell is a K562 cell or a derivative thereof. In some embodiments, the cell is a primary cell.

In some embodiments, the recombinant viral vectors are produced by a producer cell line method. Briefly, for example, a cell line (e.g., a HEK-293 cell line) is stably transfected with a plasmid containing a Rep gene, a Cap gene, and a promoter-payload sequence. Cell lines are screened to select a lead clone for recombinant vector production, which in some embodiments, are expanded to a production bioreactor and infected with, a helper polynucleotide (e.g., a wild-type adenovirus) as a helper to initiate vector production. In some embodiments, the recombinant viral vector (e.g., rAAV) is subsequently harvested, adenovirus is inactivated (e.g., by heat) and/or removed, and the viral particles are purified. In some embodiments, recombinant viral vectors are purified and formulated.

In some embodiments, suitable media is used for the production of recombinant vectors. These media comprise, without limitation, media produced by Hyclone Laboratories and JRH comprising Modified Eagle Medium (MEM), Roswell Park Memorial Institute (RPMI) 1640, Eagle's Minimal Essential Medium (EMEM), Ham's F-10 Medium, Iscove's Modified Dulbecco's Medium (IMDM), Neuralbasal Medium, Dulbecco's Modified Eagle Medium (DMEM), custom formulations, particularly with respect to custom media formulations for use in production of recombinant vectors.

In some embodiments, suitable production culture media of the present disclosure is supplemented with serum or serum-derived recombinant proteins at a level of 0.5-20 (v/v or w/v). In some embodiments, vectors are produced in serum-free conditions which are also referred to as media with no animal-derived products. In some embodiments, commercial or custom media is designed to support production of vectors, comprising supplementation of without limitation glucose, vitamins, amino acids, and or growth factors, in order to increase the titer and/or yield of vector in production cultures.

Vector production cultures comprise a variety of conditions (over a wide temperature range, for varying lengths of time, and the like) suitable to the particular host cell being utilized. Vector production cultures comprise attachment-dependent cultures which are cultured in suitable attachment-dependent vessels such as, for example, plates, flasks, cell stacks, roller bottles, hollow fiber filters, microcarriers, and packed-bed or fluidized-bed bioreactors. In some embodiments, vector production cultures comprise suspension-adapted host cells such as HeLa, HEK-293, HEK-293T, HEK-293S, CHO, NSO, PER.C6, BHK, S2 and Sf9 cells which are cultured in a variety of ways comprising, for example, spinner flasks, stirred tank bioreactors, single use bioreactors such as Cytiva Xcellerex, Sartorius, and Wave, and disposable systems.

In some embodiments, viral particles of the disclosure are harvested from vector production cultures by lysis of the host cells of the production culture or by harvest of the spent media from the production culture, provided the cells are cultured under conditions to cause release of viral particles into the media from intact cells. Suitable methods of lysing cells comprise for example multiple freeze/thaw cycles, sonication, microfluidization, and treatment with chemicals, such as detergents and/or proteases.

In a further embodiment, the viral particles are purified. The term “purified” as used herein comprises a preparation of viral particles devoid of at least some of the other components that are present where the viral particles naturally occur or are initially prepared from. Thus, for example, in some embodiments, isolated viral particles are prepared using a purification technique to enrich it from a source mixture, such as a culture lysate or production culture supernatant. In some embodiments, enrichment is measured in a variety of ways, such as, for example, by the proportion of DNase-resistant particles (DRPs) or genome copies (gc) present in a solution, or by infectivity, or measured in relation to a second, potentially interfering substance present in the source mixture, such as contaminants and impurities, comprising product-derived and process-derived impurities and the like.

In some embodiments, the vector production culture harvest is clarified to remove host cell debris. In some embodiments, the production culture harvest is clarified by filtration through a series of depth filters comprising, for example, a grade DOHC Millipore Millistak+ HC Pod Filter, a grade A1HC Millipore Millistak+ HC Pod Filter, and a 0.2 μm Filter Opticap XL 10 Millipore Express SHC Hydrophilic Membrane filter. Clarification can also be achieved by a variety of other standard techniques, such as, centrifugation or filtration through any cellulose acetate filter of 0.2 μm or greater pore size.

In some embodiments, the vector production culture harvest is further treated with a nuclease (e.g., DNA or RNA nuclease) to digest any high molecular weight DNA present in the production culture. In some embodiments, the nuclease digestion is performed under standard conditions.

In some embodiments, viral particles are isolated or purified using one or more of the following purification steps: equilibrium centrifugation; flow-through anion exchange filtration; tangential flow filtration (TFF) for concentrating the viral particles; vector capture by apatite chromatography; heat inactivation of helper virus; vector capture by hydrophobic interaction chromatography; buffer exchange by size exclusion chromatography (SEC); nanofiltration; and vector capture by anion exchange chromatography, cation exchange chromatography, or affinity chromatography. In some embodiments, these steps are used alone, in various combinations, or in different orders. In some embodiments, the method comprises all the steps in the order as described below.

Described herein, are methods for generating a recombinant vector, wherein the methods comprise providing cells transfected with a helper polynucleotide. In some embodiments, the cells are transfected with a helper polynucleotide that provides helper functions to the AAV. In some embodiments, the helper polynucleotide provides adenovirus functions, comprising, e.g., E1A, E1B, E2A, E4ORF6, and/or VA RNA. The sequences of adenovirus genes providing these functions, in some embodiments, are obtained from any known adenovirus serotype, such as serotypes 2, 3, 4, 5, 7, 12 and 40, and further comprising any of the presently identified human types. In some embodiments, the adenovirus functions are provided by a self-attenuating adenovirus. In some embodiments, the methods involve transfecting the cell with vectors expressing one or more genes necessary for AAV replication, AAV gene transcription, and/or AAV packaging.

Described herein, are methods for generating a recombinant vector, wherein the methods comprise providing cells transfected with a helper polynucleotide that is under the control of a promoter. In some embodiments, the cells are stable host cells comprising the required component(s) under the control of an inducible promoter. In some embodiments, the cells are stable host cells comprising the required component(s) under the control of a constitutive promoter. In some embodiments, the cells are stable host cells comprising the selected components under the control of a constitutive promoter and other selected components under the control of one or more inducible promoters. For example, a stable host cell is generated which is derived from HEK-293 cells (which contain E1 helper functions under the control of a constitutive promoter), but which contains the Rep and/or Cap proteins under the control of inducible promoters.

In some embodiments, the minigene, Rep sequences, Cap sequences, and helper functions required for producing the rAAV of the disclosure are delivered to the packaging host cell in the form of any genetic element which transfers the sequences. In some embodiments, the selected genetic elements are delivered by any suitable method.

Production of recombinant vectors, such as rAAV vectors, in some embodiments, comprises transfection, stable cell line production, and infectious hybrid virus production systems which comprise adenovirus-AAV hybrids, herpesvirus-AAV hybrids, bocavirus-AAV hybrids, and baculovirus-AAV hybrids.

Methods comprising transfection, stable cell line production, and infectious hybrid virus production systems, in some embodiments, are used for production of rAAV vectors. In some embodiments, the methods described herein comprise adenovirus-AAV hybrids, herpesvirus-AAV hybrids, bocavirus-AAV hybrids, and baculovirus-AAV hybrids. rAAV production cultures for the production of rAAV virus particles all require; 1) suitable host cells, comprising, for example, human-derived cell lines such as HeLa, A549, or HEK-293 cells, or HEK-293T cells, or HEK-293S cells, or insect-derived cell lines such as Sf9, in the case of baculovirus production systems, or transgenic plant cells; 2) suitable helper virus function, provided by wild-type or mutant adenovirus (such as temperature-sensitive adenovirus), herpes virus, baculovirus, or a plasmid construct providing helper functions; 3) AAV Rep and Cap genes and gene products; 4) a desired genomic payload (such as a heterologous sequence encoding any desired sequence or fragment or variant thereof) flanked by at least one AAV ITR sequence; and 5) suitable media and media components to support rAAV production. Suitable media for the production of rAAV vectors comprise, without limitation, media produced by Hyclone Laboratories and JRH comprising Modified Eagle Medium (MEM), Roswell Park Memorial Institute (RPMI) 1640, Eagle's Minimal Essential Medium (EMEM), Ham's F-10 Medium, Iscove's Modified Dulbecco's Medium (IMDM), Neuralbasal Medium, Dulbecco's Modified Eagle Medium (DMEM).

In some embodiments, recombinant AAV particles are generated by transfecting producer cells with a plasmid (cis plasmid) containing a rAAV genome comprising a payload flanked by one or more AAV ITRs and a separate construct expressing the AAV Rep and Cap genes in trans. In some embodiments, adenovirus helper factors such as E1A, E1B, E2A, E4ORF6 and VA RNAs are provided by either adenovirus infection or by transfecting a third plasmid providing adenovirus helper polynucleotides into the producer cells. In some embodiments, producer cells are HEK-293 cells. In some embodiments, the helper polynucleotides provided will vary depending on the producer cells used and whether the producer cells already carry some of these helper polynucleotides.

In some embodiments, the rAAV vectors described herein are produced by a triple transfection method, such as the exemplary triple transfection method provided infra. In some embodiments, the plasmid containing a Rep gene and a Cap gene, along with a helper adenoviral plasmid, is transfected (e.g., using squeeze-poration, lipofection, optical transfection, the calcium phosphate method, or polyethylenimine (PEI)) into a cell line (e.g., HEK-293 cells), and virus is collected and optionally purified.

In some embodiments, rAAV vectors are produced by a producer cell line method, such as the exemplary producer cell line method provided infra. Briefly, a cell line (e.g., a HEK-293 cell line) is stably transfected with a plasmid containing a Rep gene, a Cap gene, and a promoter-payload sequence. Cell lines are screened to select a lead clone for rAAV production, which are then expanded to a production bioreactor and infected with an adenovirus (e.g., a wild-type adenovirus) as helper to initiate rAAV production. Virus is subsequently harvested, adenovirus may be inactivated (e.g., by heat) and/or removed, and the rAAV vectors are purified.

In some embodiments, a method is provided for producing any rAAV vector as disclosed herein comprising (a) culturing a host cell under a condition that rAAV vectors are produced, wherein the host cell comprises (i) one or more AAV packaging genes, wherein each said AAV packaging gene encodes an AAV replication and/or encapsidation protein; (ii) a rAAV pro-vector comprising nucleic acids encoding a therapeutic polypeptide and/or nucleic acid as described herein flanked by at least one AAV ITR, and (iii) an AAV helper function; and (b) recovering the rAAV vectors produced by the host cell. In some embodiments, said at least one AAV ITR is selected from the group consisting of AAV ITRs are AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, AAV-HSC16, a goat AAV, bovine AAV, or mouse AAV or the like. In some embodiments, the replication protein is an AAV2 replication protein.

### Methods of Treating Cancer

Described herein are methods of treating cancer in a subject in need thereof, comprising: administering to the subject a rAAV described herein. In some embodiments, the rAAV comprises a minigene comprising a splice modulator binding site. In some embodiments, the methods of treating cancer in a subject in need thereof further comprises administering a small molecule splice modulator.

Administration is not limited to any particular route, but rather can refer to any route accepted as appropriate by the medical community. In some embodiments, administration is intravenous (IV), intramuscular (IM), intra-arterial, inhalation, intramedullary, intrathecal (IT), intracisternal magna (ICM), intracerebroventricular (ICV), intraparenchymal, intranasal, subcutaneous (SQ), transdermal, intraventricular, intraperitoneal (IP), intragastric (IG), mucosal, buccal, enteral, intravitreal, and/or through a portal vein catheter; and/or combinations of any of the foregoing. In some embodiments, rAAV vector compositions described herein are locally administered to a diseased tissue (e.g., a tumor).

In some embodiments, rAAV vector compositions provided by the present disclosure are administered in combination with a splicing modifier or modulator. In some embodiments, rAAV vector compositions are provided sequentially with a splicing modifier. In some embodiments, rAAV vector compositions are locally administered directly to a diseased tissue and a splicing modifier is administered via a different route. In some embodiments, rAAV vector compositions are locally administered directly to a diseased tissue and a splicing modifier is administered orally. In some embodiments, rAAV vector compositions are locally administered directly to a diseased tissue and a splicing modifier is administered systemically. In some embodiments, both rAAV vector composition and splicing modifier are locally administered to a diseased tissue.

In some embodiments, the subject is human. In some embodiments, the subject has cancer. In some embodiments, the subject has a glioma, such as a grade III or grade IV glioma (glioblastoma). In some embodiments, the subject has glioblastoma. In some embodiments, the subject has a primary tumor. In some embodiments, the subject has a metastasis. In some embodiments, the subject has a metastatic brain tumor. In some embodiments, the subject has uveal melanoma.

In some embodiments, the administration is local administration directly to a diseased tissue (e.g., a tumor). In some embodiments, the administration is to the central nervous system. In some embodiments, the administration is to a brain. In some embodiments, the administration is to a brain ventricle. In some embodiments, the administration is intratumoral.

Any suitable route of administration or combination of different routes can be used, including systemic administration (e.g., intravenous, intravascular, intraarterial), local injection into the central nervous system (CNS; e.g., intratumoral injection, intracranial injection, intracerebral injection, intracerebroventricular, intraparenchymal, or injection into the cerebrospinal fluid (CSF) via the cerebral ventricular system, cisterna magna, or intrathecal space), or local injection at other bodily sites (e.g., intraocular, intranasal, intramuscular, subcutaneous, intradermal injection, transdermal). In some embodiments, the administration is by intratumoral injection, intracranial injection, intracerebral injection, intracerebroventricular, or injection into the cerebrospinal fluid (CSF) via the cerebral ventricular system, cisterna magna, or intrathecal space.

In some embodiments, intracerebroventricular injection occurs in the right lateral ventricle, left lateral ventricle, third ventricle, fourth ventricle, interventricular foramina (also called the foramina of Monro), cerebral aqueduct, central canal, median aperture, right lateral aperture, left lateral aperture, perivascular space, or the subarachnoid space.

Administration can be performed by use of an osmotic pump, by electroporation, or by other means. In some embodiments, administration of the rAAV vectors described herein are performed before, after, or simultaneously with surgical tumor removal or biopsy.

In some embodiments, the administration is by Convection Enhanced Delivery (CED). CED uses direct infusion of a drug-containing liquid into tissue so that transport is dominated by convection. In some embodiments, the device is an osmotic pump. In some embodiments, the device is an infusion pump. In some embodiments, CED is performed with a step-design cannula.

In some embodiments, magnetic resonance imaging (MRI) guided CED is performed to deliver the rAAV vectors of the present disclosure. In some embodiments, CED further comprises the use of a tracing agent. In some embodiments, the tracing agent is an MRI contrast enhancing agent. In some embodiments, the MRI contrast enhancing agent is gadolinium and related chemical derivatives. In some embodiments, the MRI contrast enhancing agent and the rAAV vectors are administered simultaneously. In some embodiments, the MRI contrast enhancing agent is mixed with the rAAV vectors directly prior to administration.

### Pharmaceutical Compositions

Described herein, in certain embodiments, are pharmaceutical compositions comprising (a) the polynucleotides of the disclosure, the plasmids of the disclosure, or rAAV comprising the polynucleotides of the disclosure, and (b) a pharmaceutically acceptable excipient. A pharmaceutical composition disclosed herein is in a form suitable for administration to an individual in need thereof.

In some embodiments, the pharmaceutically acceptable excipients of the disclosure are suitably selected from the group consisting of an injectable excipient liquid such as sterile water for injection; and an aqueous solution such as saline.

Acceptable excipients are physiologically acceptable to the administered subject and retain the therapeutic properties of the compounds with/in which it is administered. Acceptable excipients and their formulations are and generally described in, for example, Remington's Pharmaceutical Sciences, supra. One exemplary excipient is physiological saline. The phrase “pharmaceutically acceptable excipient” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each excipient is acceptable in the sense of being compatible with the other ingredients of the formulation and not injurious to a subject to whom it is administered. Nor should an acceptable excipient alter the specific activity of the subject compounds.

In another embodiment, pharmaceutical compositions disclosed herein further comprise an acceptable additive to improve the stability of the compounds in composition and/or to control the release rate of the compositions. Acceptable additives do not alter the specific activity of the subject compounds. Exemplary acceptable additives include, but are not limited to, a sugar such as mannitol, sorbitol, glucose, xylitol, trehalose, sorbose, sucrose, galactose, dextran, dextrose, fructose, lactose, and mixtures thereof. Acceptable additives are combined with acceptable carriers and/or excipients such as dextrose in some embodiments. Alternatively, exemplary acceptable additives include, but are not limited to, a surfactant such as polysorbate 20 or polysorbate 80 to increase stability of the peptide and decrease gelling of the solution. In some embodiments, the surfactant is added to the composition in an amount of 0.01% to 5% of the solution. Addition of such acceptable additives increases the stability and half-life of the composition in storage.

Suspensions, lyophilized, and crystal forms of the rAAV vectors, polynucleotides, or compositions herein are also contemplated herein; methods to make suspensions, lyophilizations, and crystal forms are known to one of skill in the art.

In some embodiments, pharmaceutical compositions disclosed herein are sterile. In some embodiments, pharmaceutical compositions disclosed herein are sterilized by conventional, well known sterilization techniques. For example, sterilization is readily accomplished by filtration through sterile filtration membranes. In some embodiments, the resulting solution is packaged for use or filtered under aseptic conditions and lyophilized, the lyophilized preparation being combined with a sterile solution prior to administration.

Freeze-drying is employed to stabilize polypeptides for long-term storage, such as when a polypeptide is relatively unstable in liquid compositions, in some embodiments.

In some embodiments, some excipients such as, for example, polyols (including mannitol, glycerol, sorbitol and it's derivatives like Polysorbate 20); salts (including NaCl, MgCl₂, and KCl), sugars (including glucose, sucrose, and trehalose); surfactants (including poloxamer 188), and amino acids (including alanine, glycine, and glutamic acid), act as stabilizers for freeze-dried products. Polyols, surfactants, and sugars are also used to protect polypeptides from freezing and drying-induced damage and to enhance the stability during storage in the dried state in some embodiments. Sugars are, in some embodiments, effective in both the freeze-drying process and during storage. Other classes of molecules, including mono- and disaccharides and polymers such as PVP, have also been reported as stabilizers of lyophilized products.

For injection, in some embodiments, pharmaceutical compositions disclosed herein are in a powder suitable for reconstitution with an appropriate solution as described above. Examples of these include, but are not limited to, freeze-dried, rotary-dried or spray-dried powders, amorphous powders, granules, precipitates, or particulates. For injection, the compositions optionally contain stabilizers, pH modifiers, surfactants, bioavailability modifiers and combinations of these.

Sustained-release preparations are prepared in some embodiments. Suitable examples of sustained-release preparations include semipermeable matrices of solid hydrophobic polymers containing pharmaceutical compositions herein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (see, e.g., U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

In some embodiments, pharmaceutical compositions disclosed herein are designed to be short-acting, fast-releasing, long-acting, or sustained-releasing as described herein. In one embodiment, pharmaceutical compositions disclosed herein are formulated for controlled release or for slow release.

In some embodiments, the pharmaceutical compositions are comprised in a container, pack, or dispenser together with instructions for administration.

### EXAMPLES

Below are examples of specific embodiments for carrying out the present disclosure. The examples are offered for illustrative purposes only and are not intended to limit the scope of the present disclosure in any way. Efforts have been made to ensure accuracy with respect to numbers used (e.g., amounts, temperatures, etc.), but some experimental error and deviation should, of course, be allowed for.

### Example 1. HEK-293T Expression of Inducible AAV-GFP

The present example describes an exemplary regulatory minigene for use in a rAAV vector of the present disclosure. The exemplary minigene was tested in HEK-293T cells, and used to control the expression of a GFP reporter polypeptide.

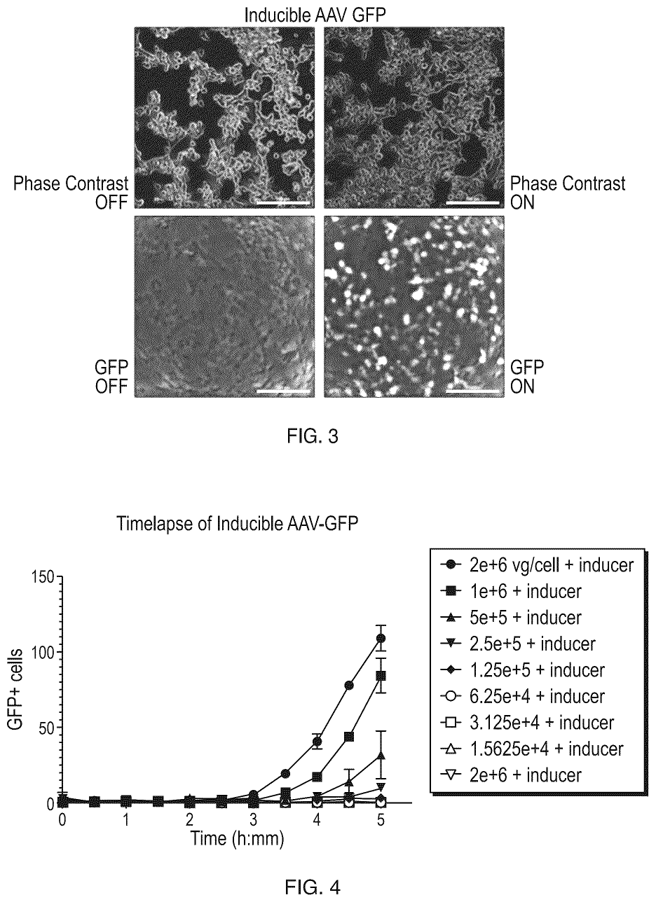

Adherent human HEK-293T cells were transduced with inducible AAV-GFP, followed by fluorescent imaging for GFP expression pre- and post-administration of an inducer molecule (e.g., LMI070). The data is seen in FIG. 3.

Adherent human HEK-293T cells were transduced with inducible AAV-GFP at varying dosages, followed by administration of an inducer molecule (e.g., LMI070), and monitored for GFP expression over several hours to determine initial expression kinetics at different AAV dosages in vitro. The data is seen in FIG. 4.

### Example 2: Engineered rAAV Vectors for Interferon Delivery

The present example describes exemplary engineered nucleic acids, provided by the present disclosure, that are useful for producing rAAV vectors capable of delivering of an interferon to a subject in need thereof.

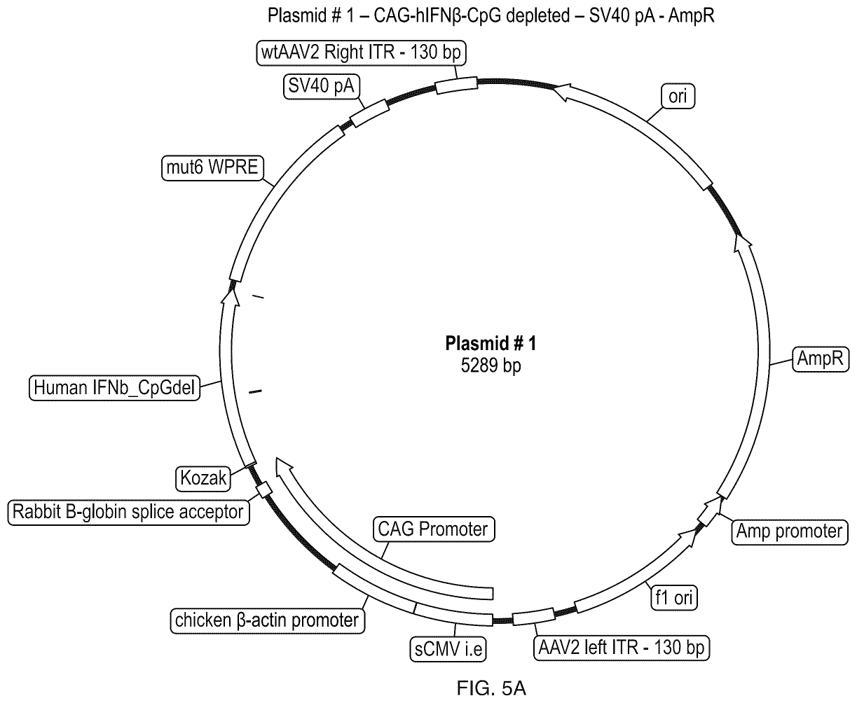

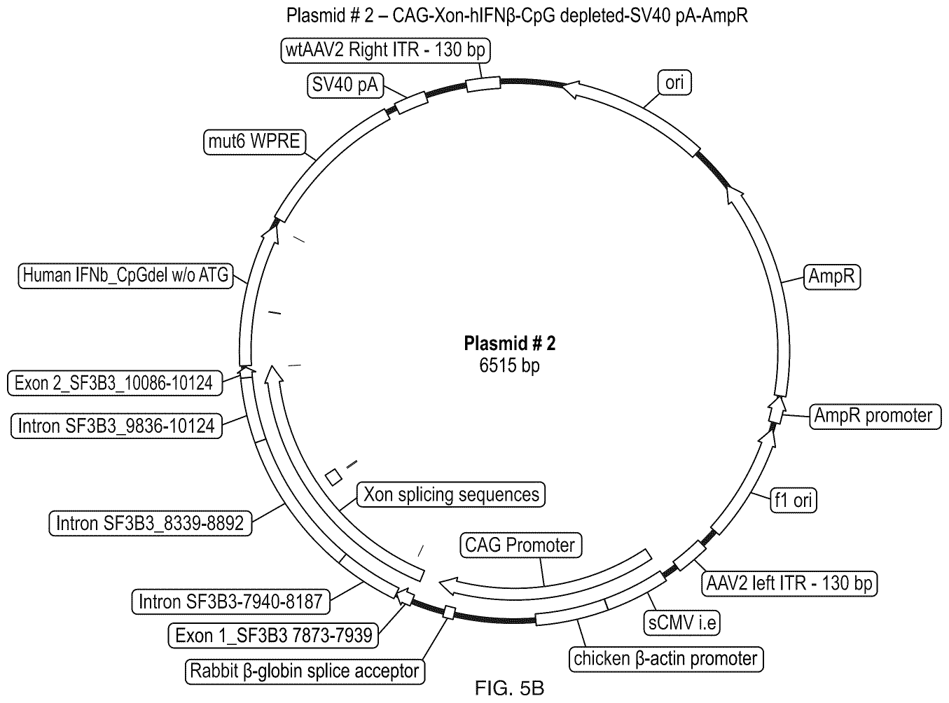

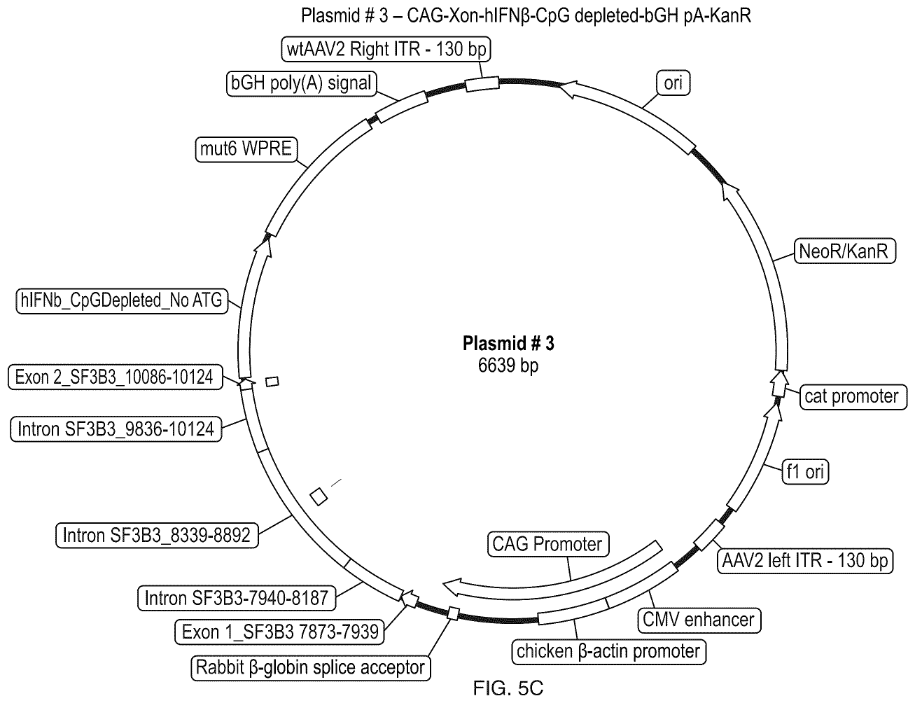

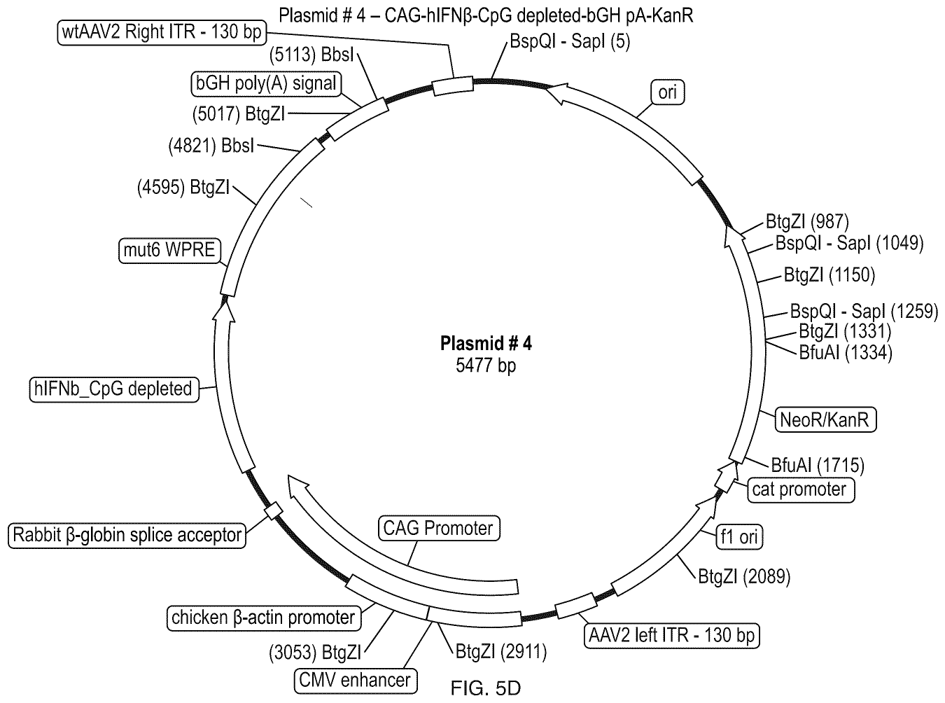

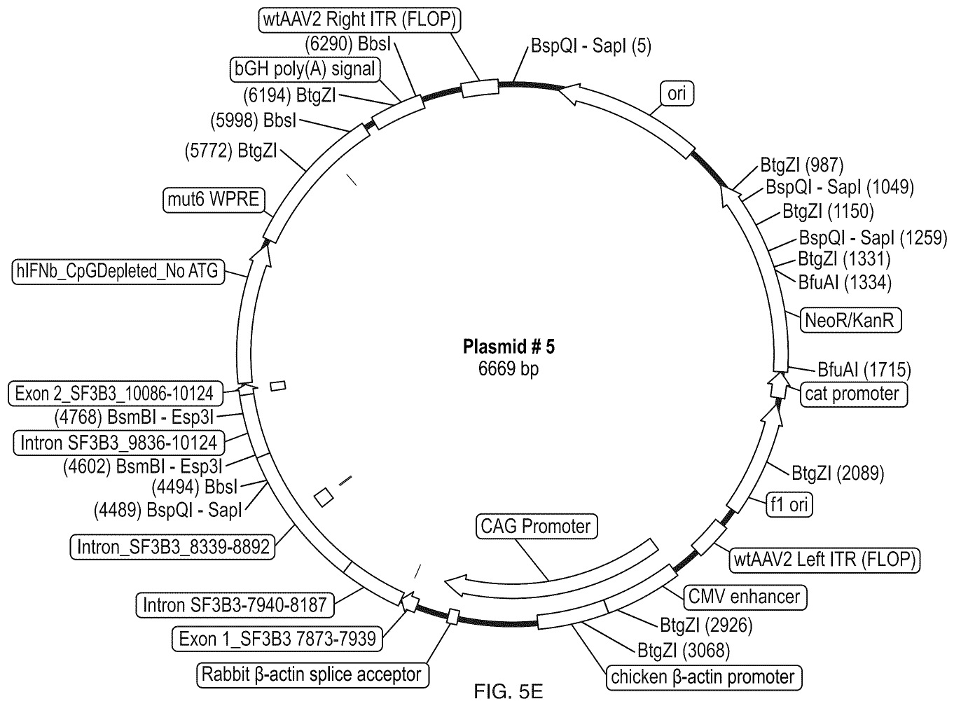

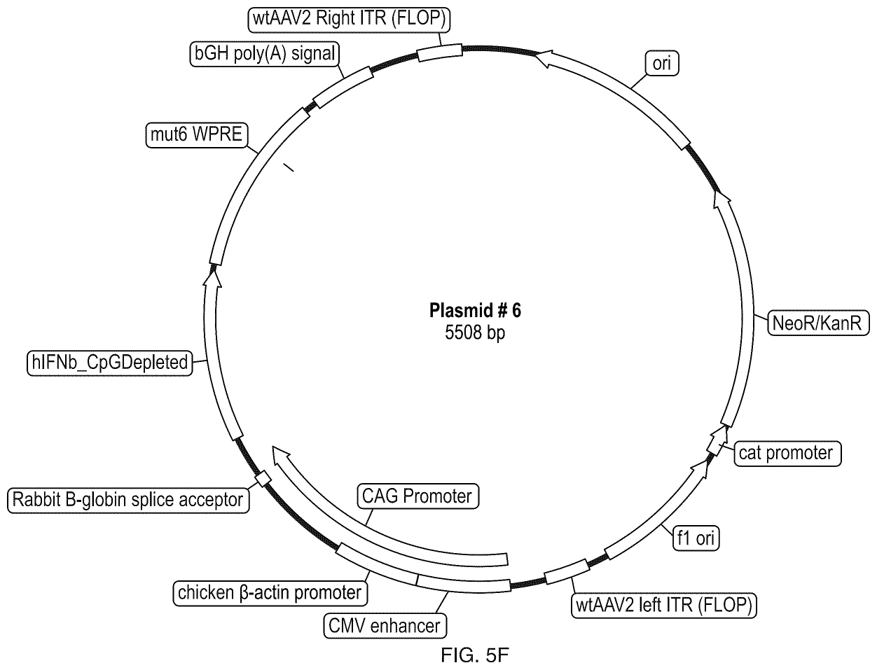

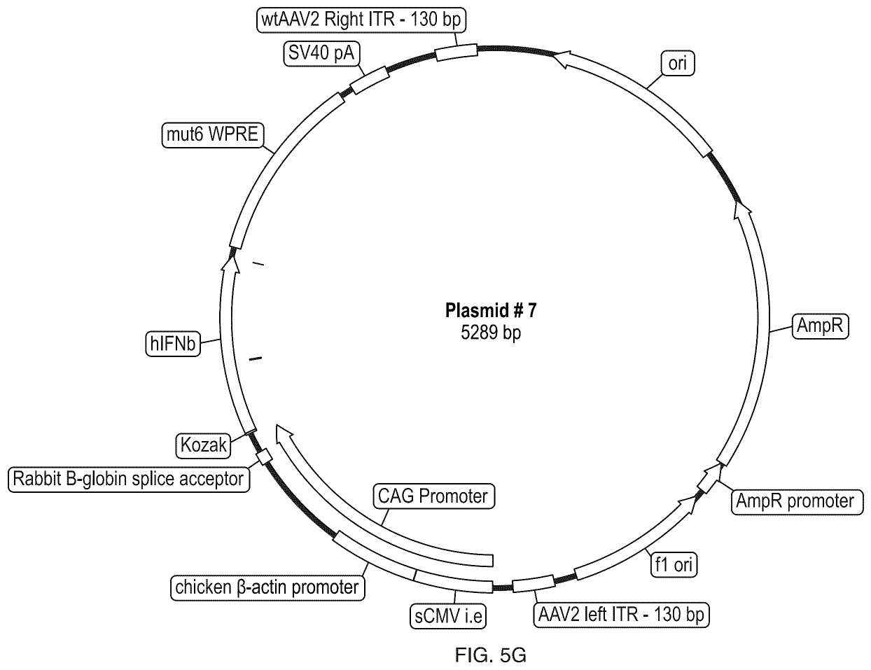

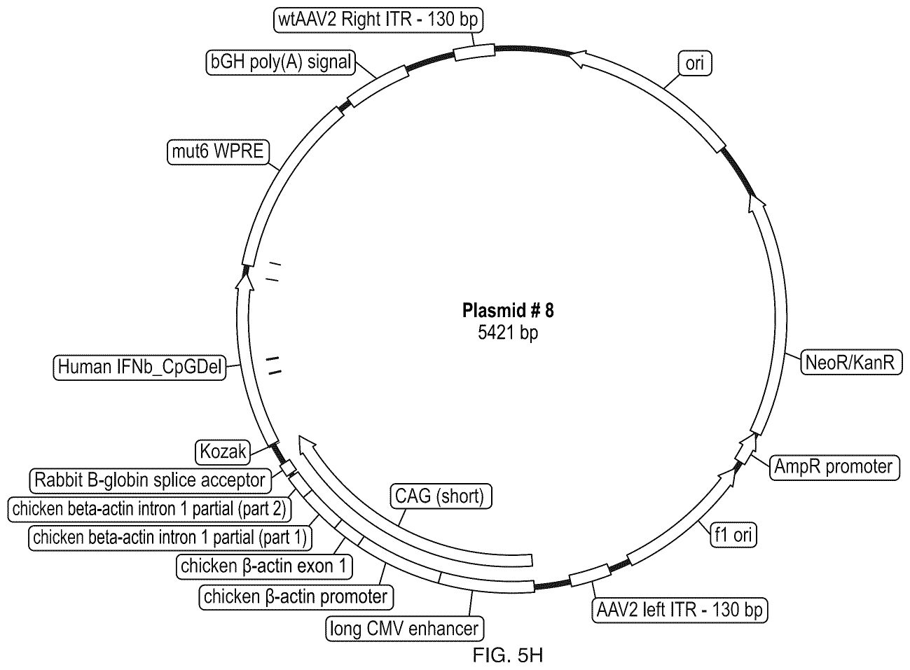

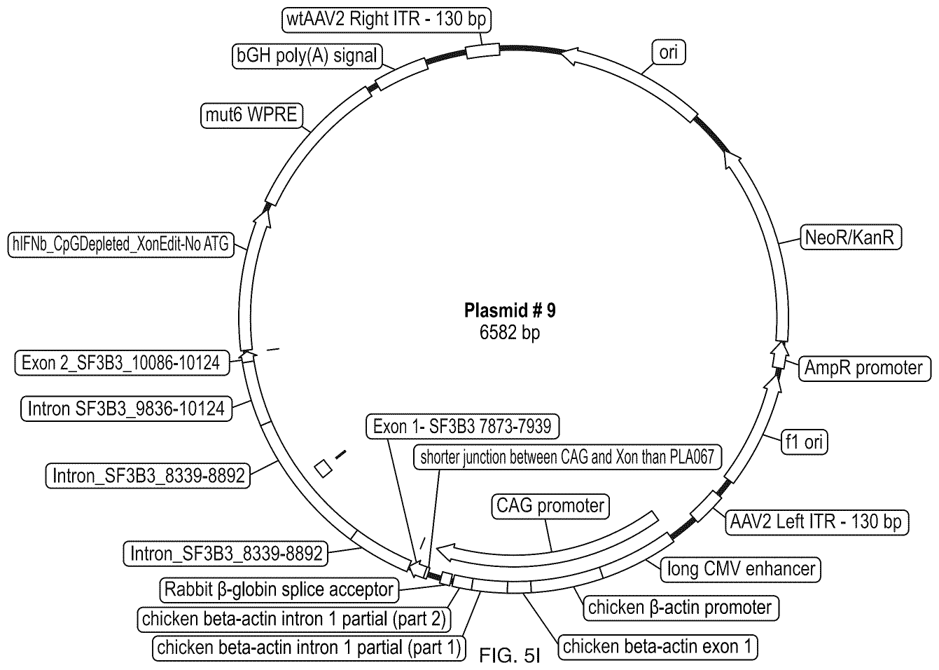

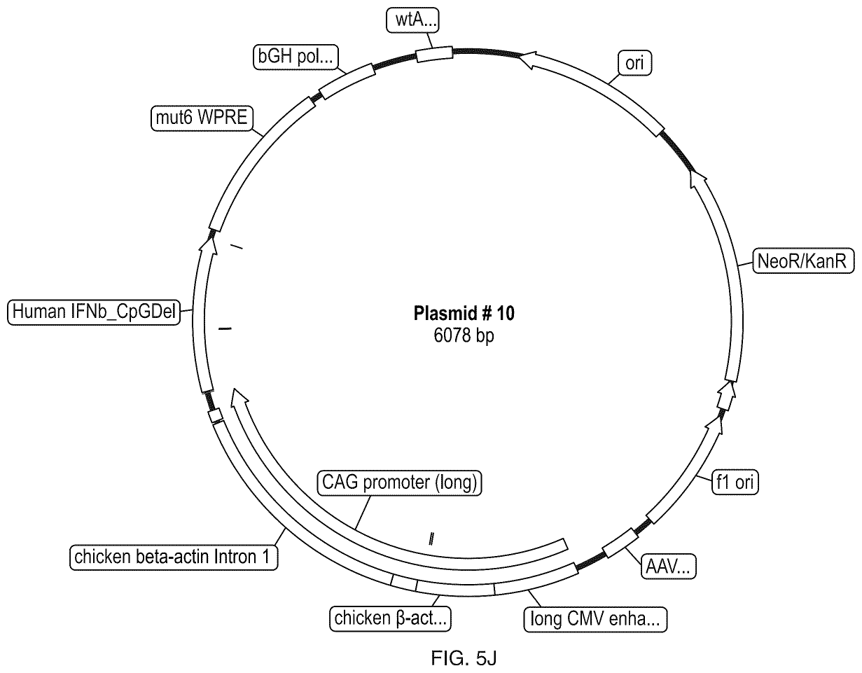

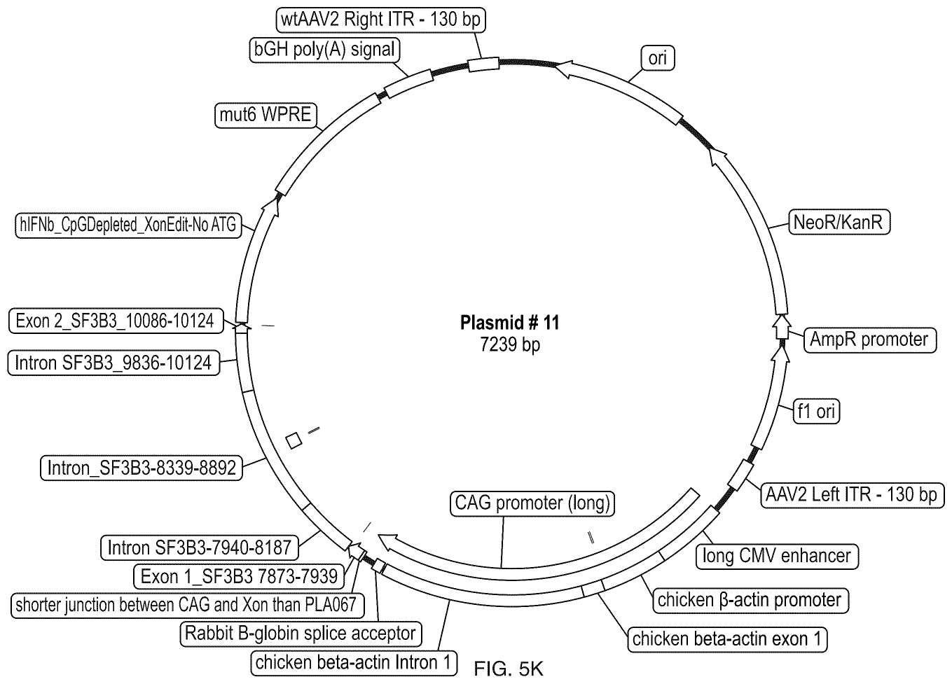

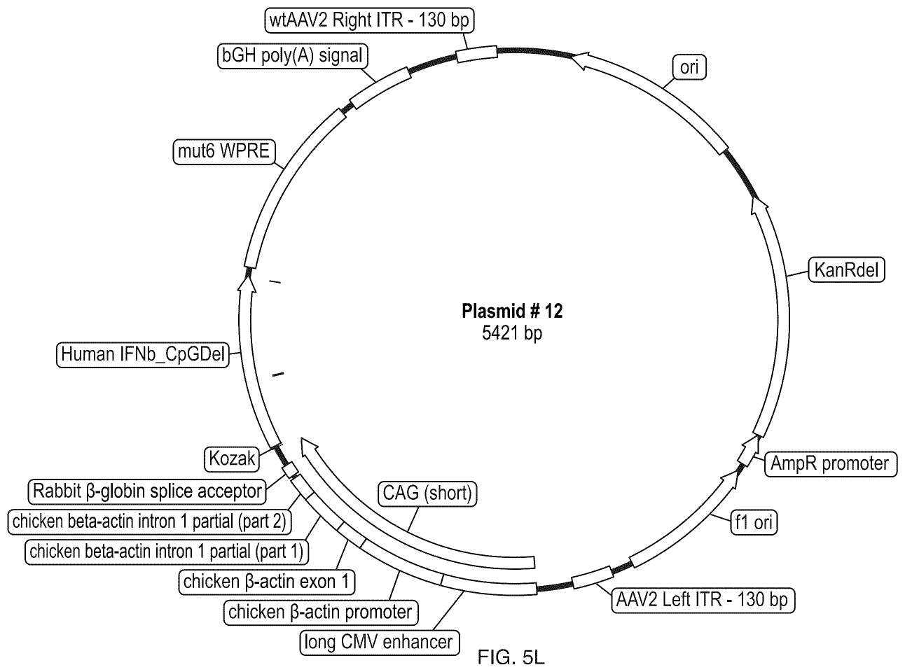

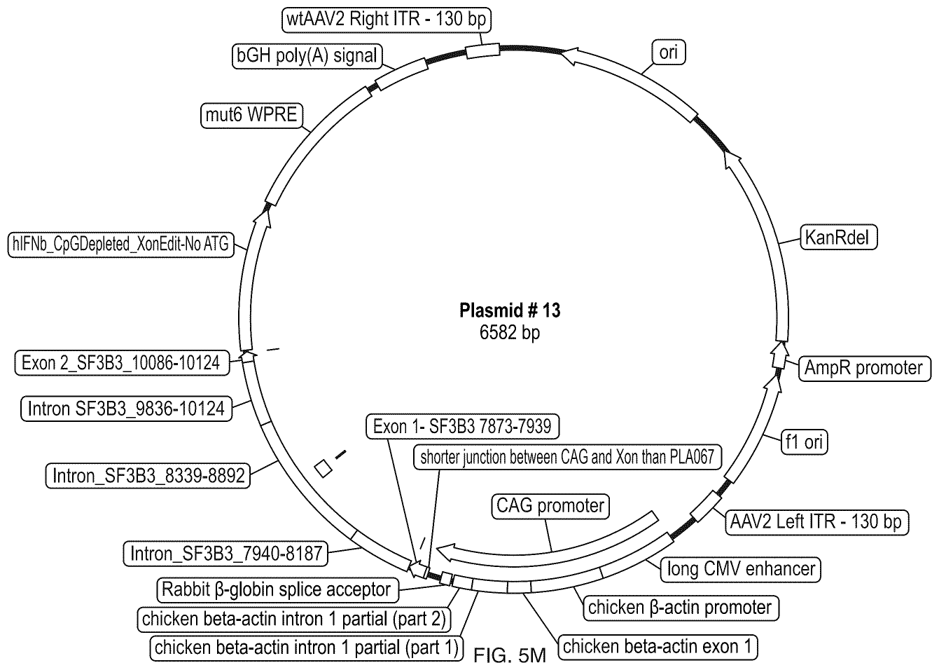

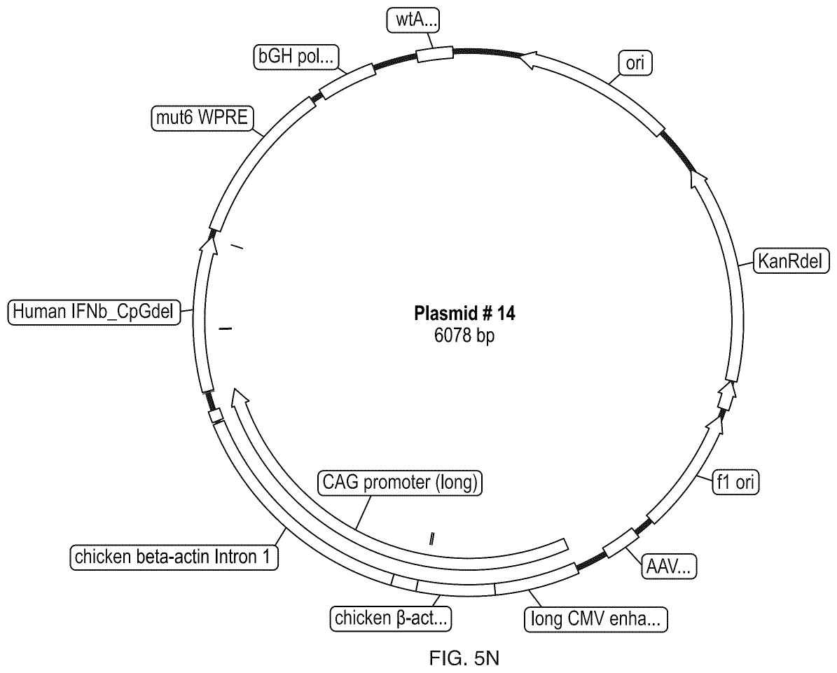

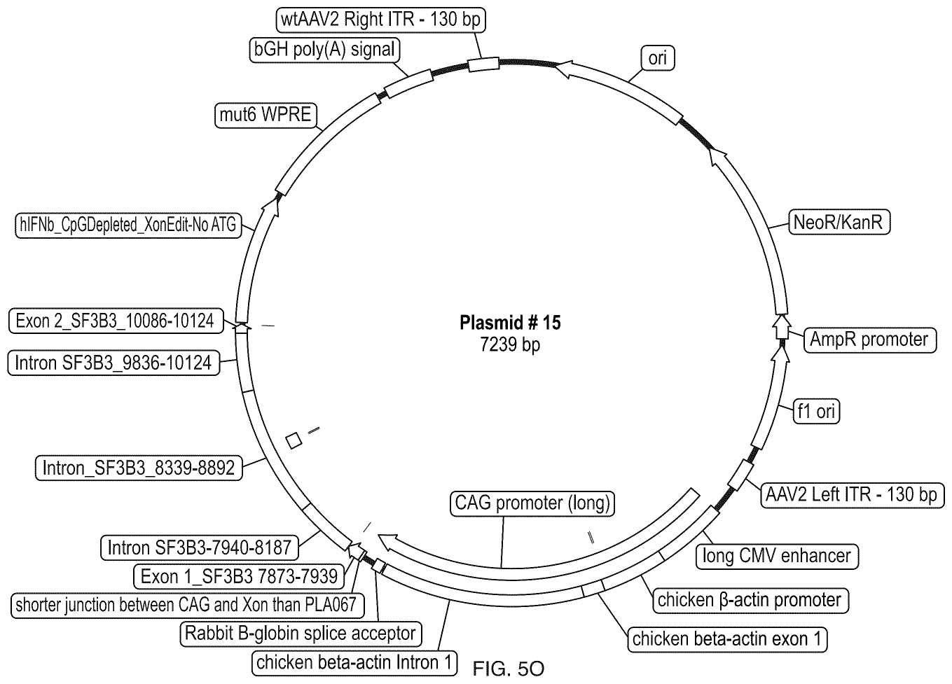

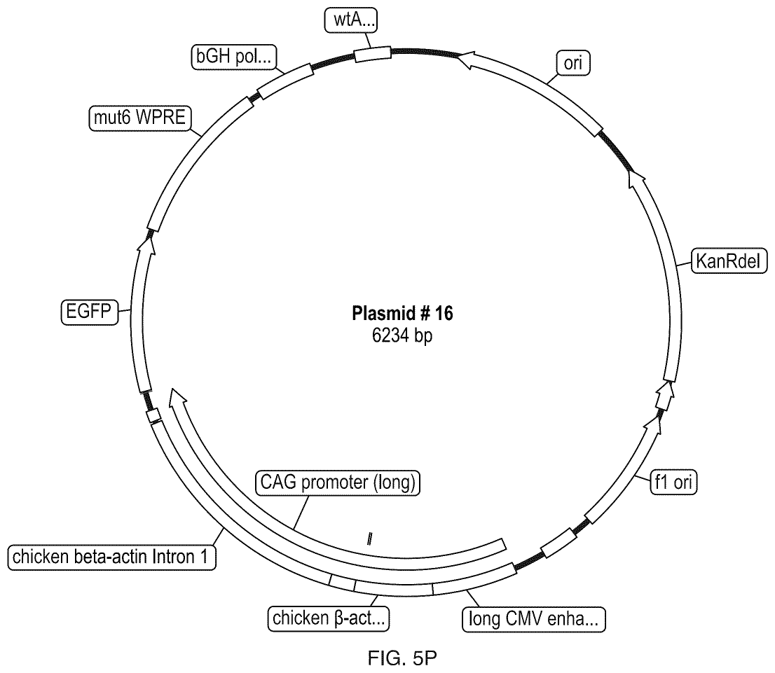

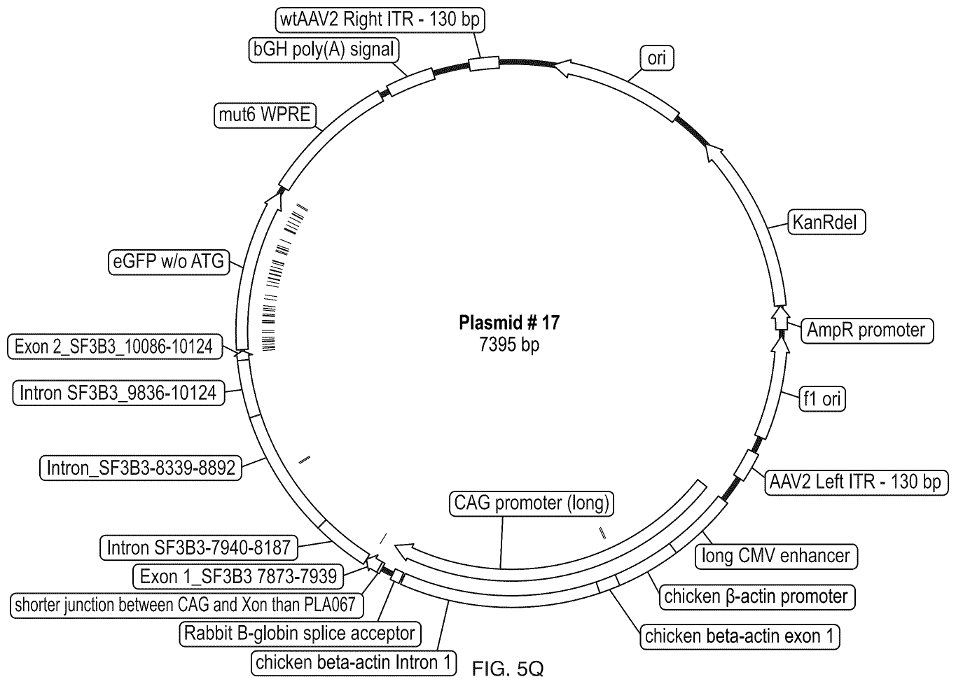

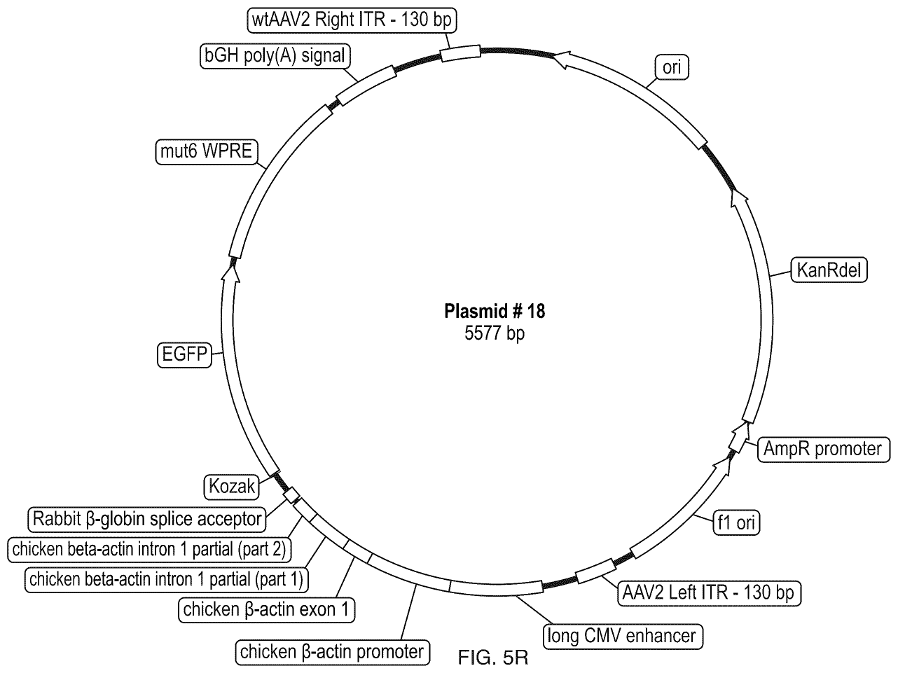

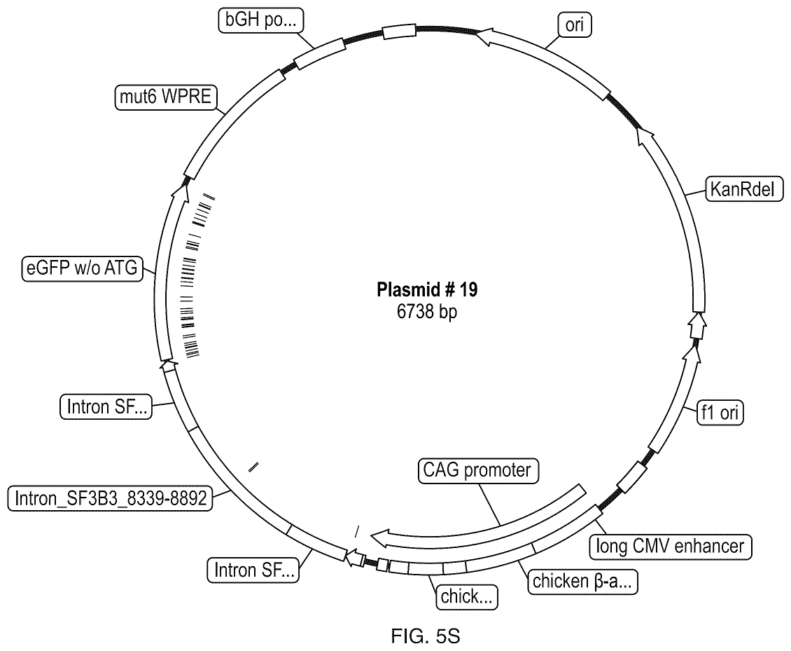

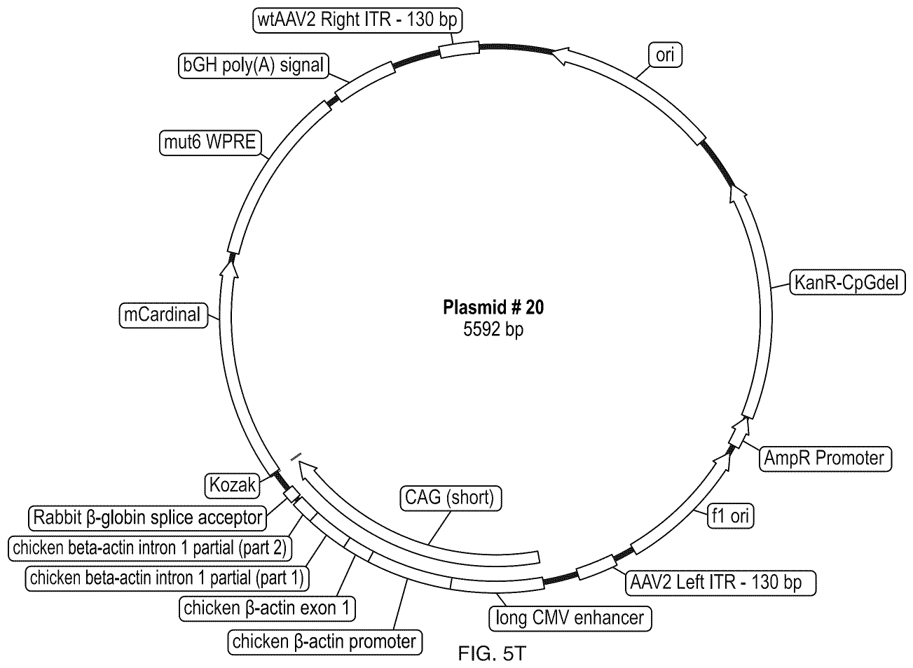

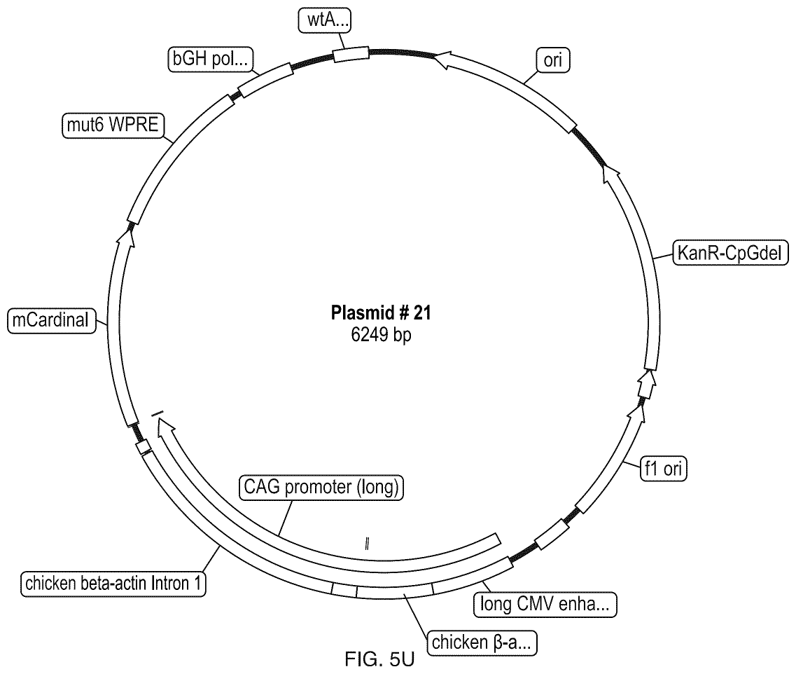

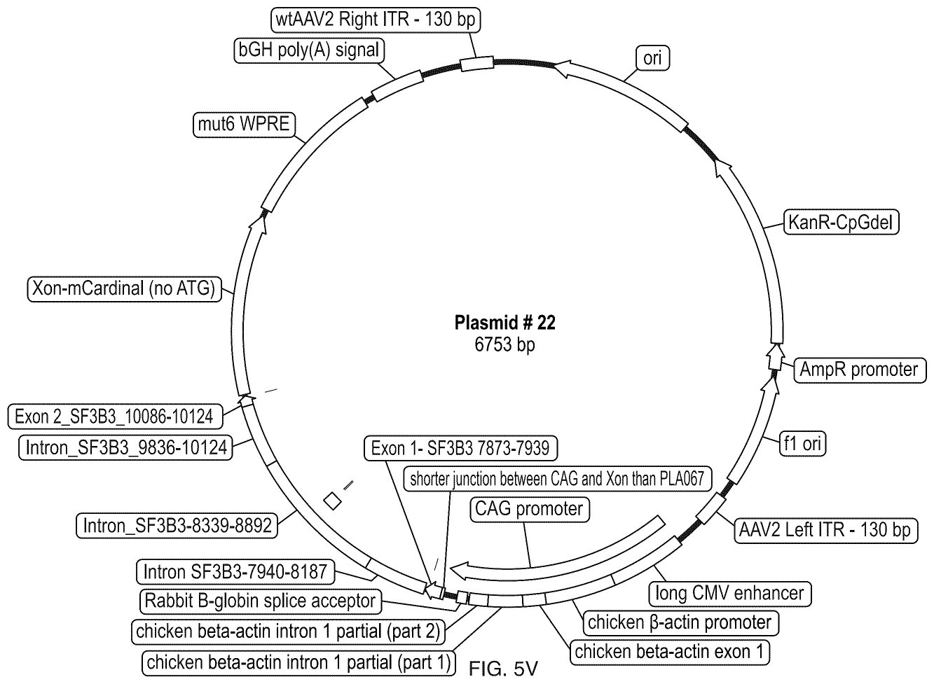

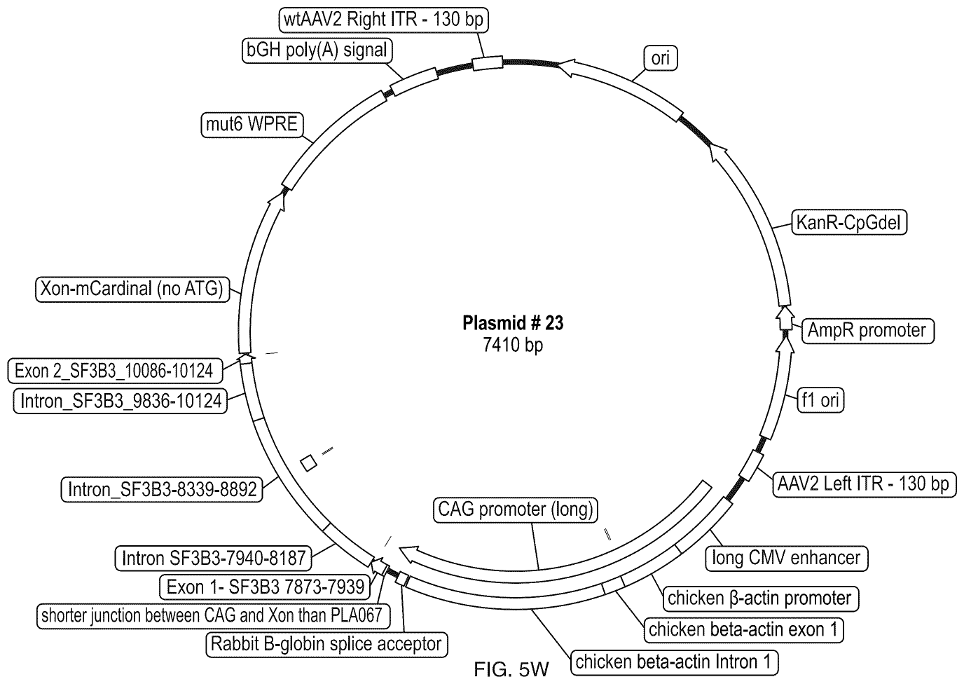

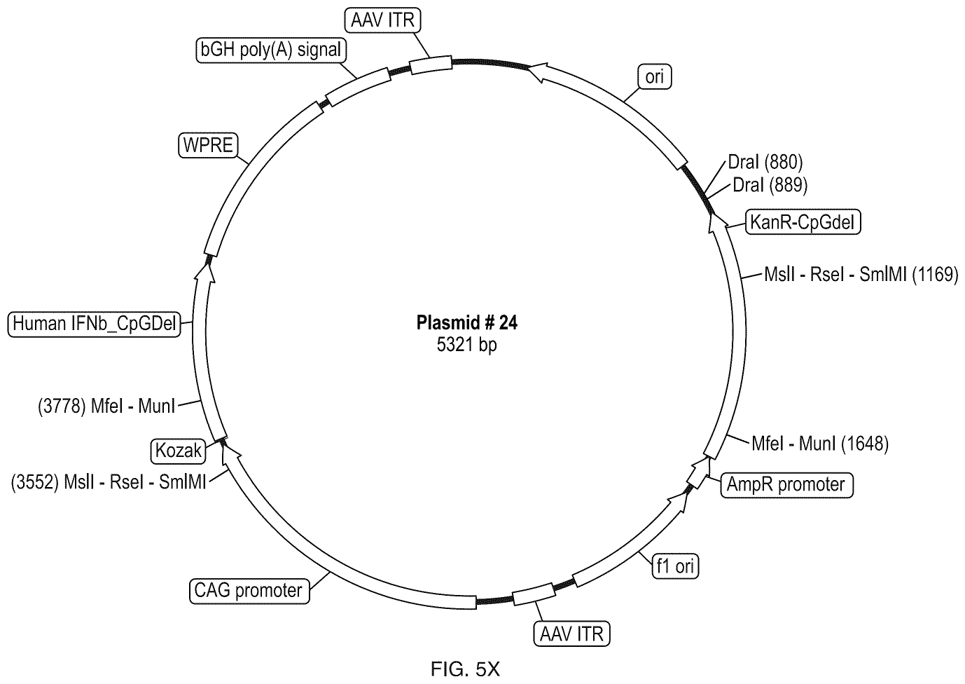

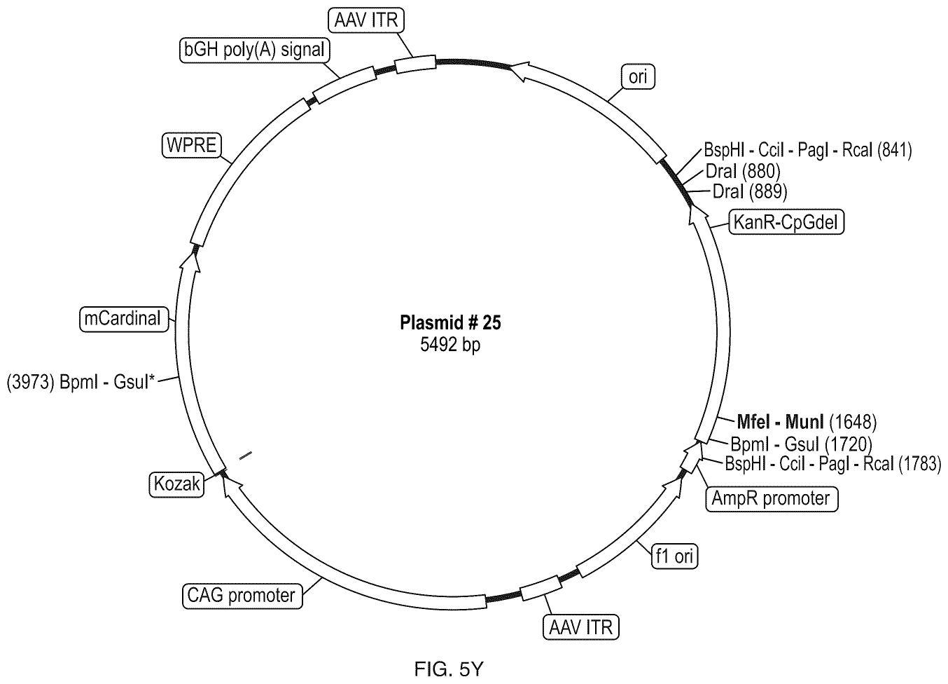

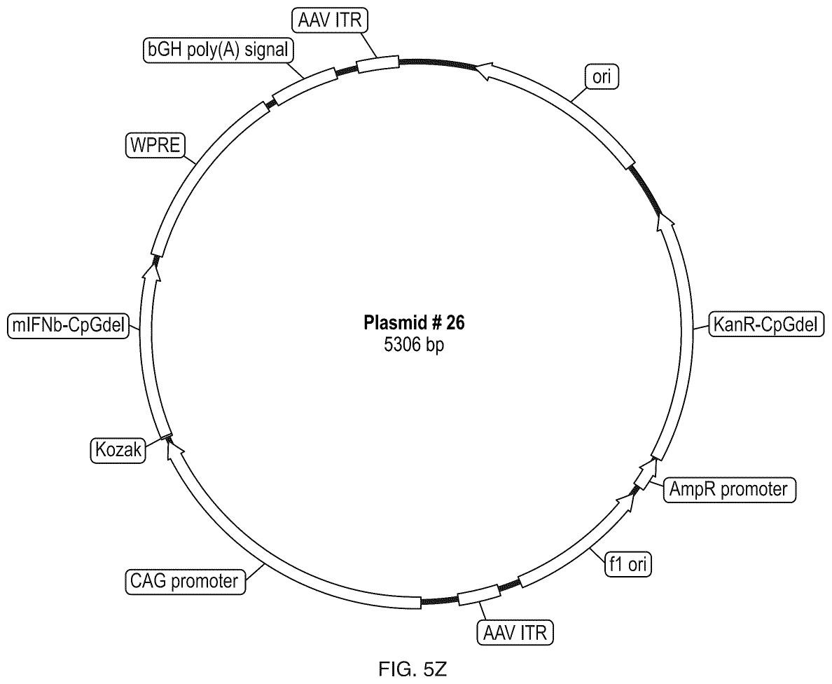

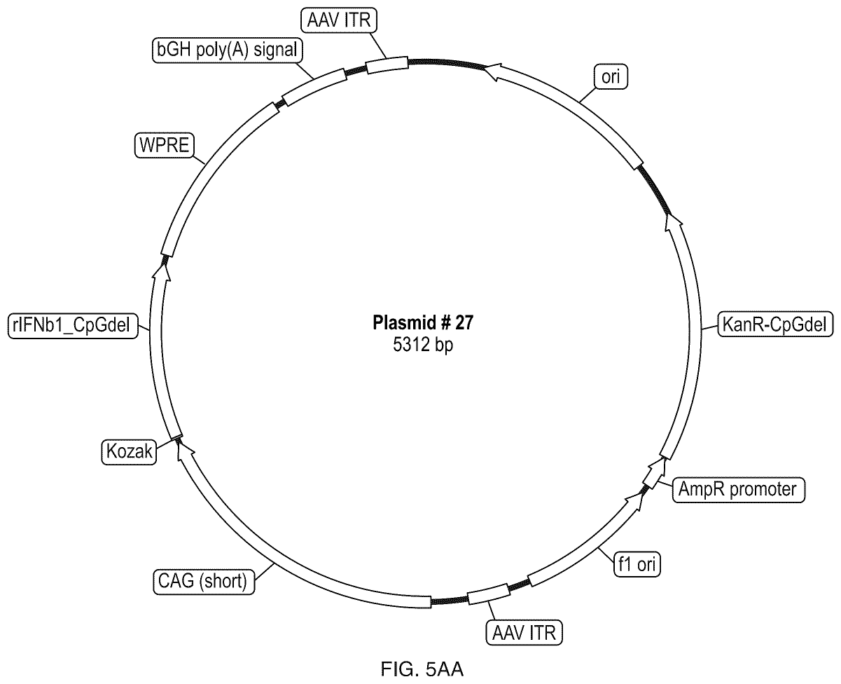

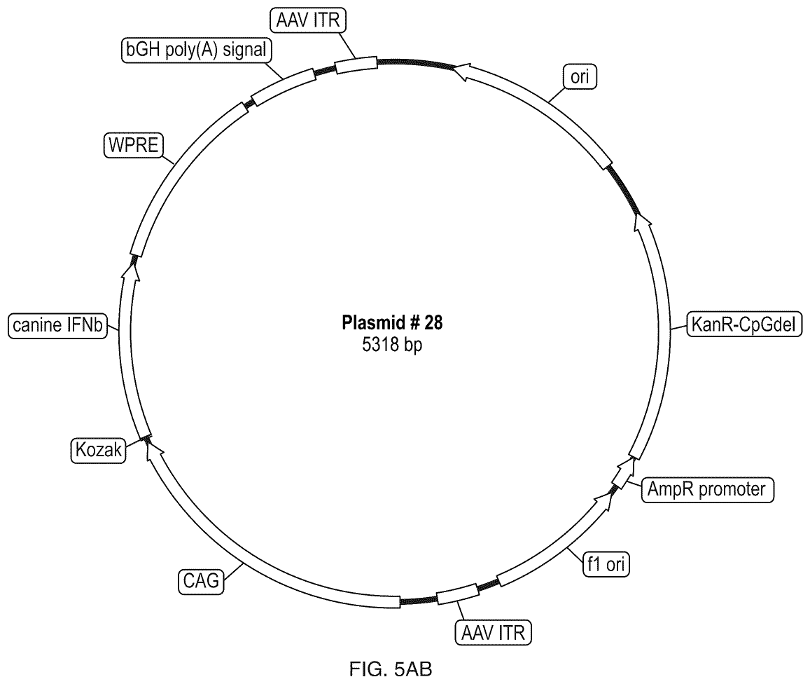

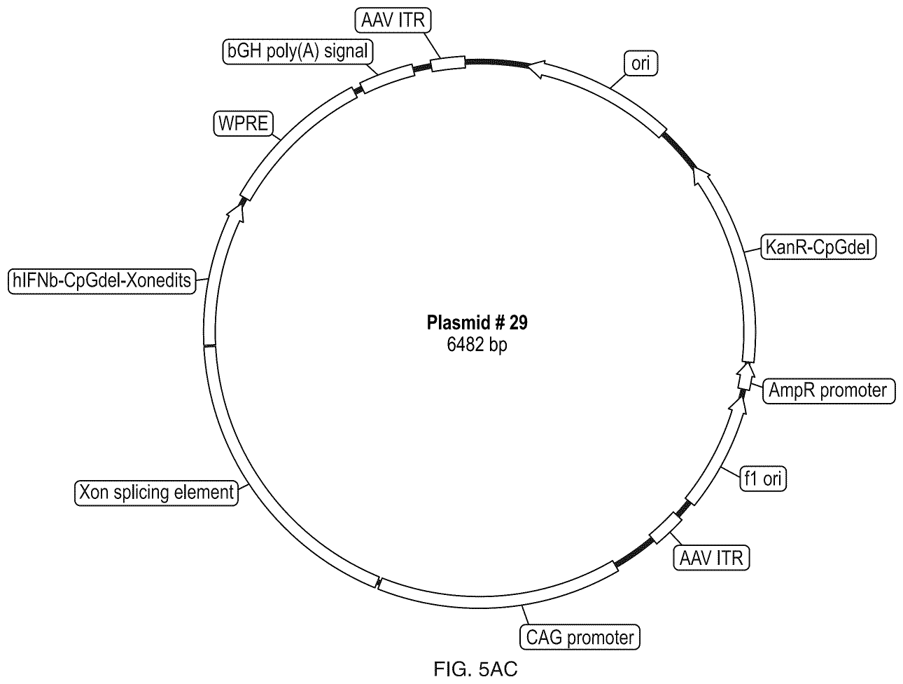

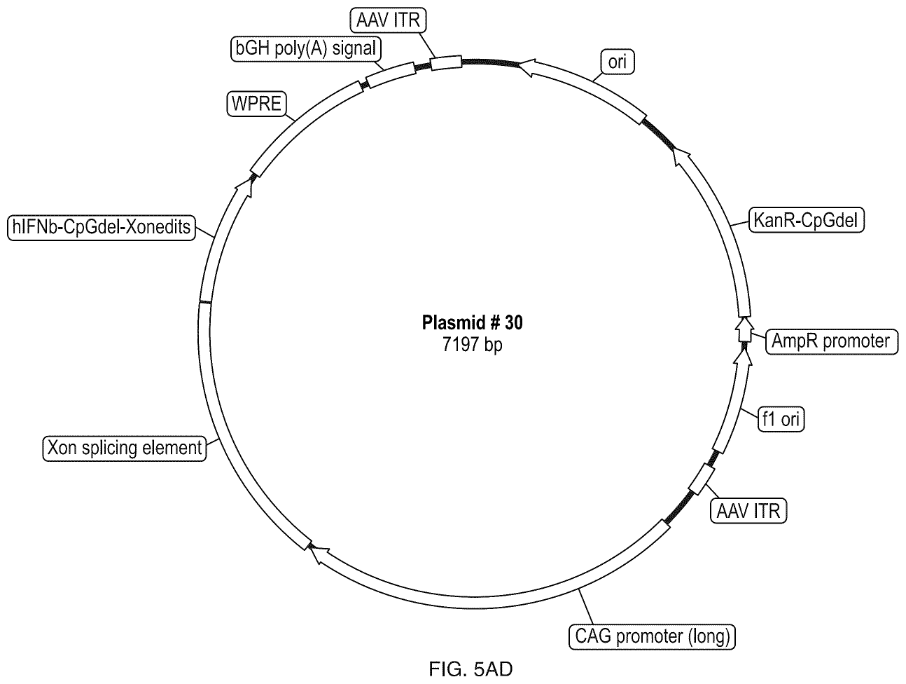

As described herein, recombinant adeno-associated viral (AAV) vectors of the present disclosure may include nucleic acid elements, including but not limited to, a promoter, a minigene, a polynucleotide comprising a coding sequence for a payload of interest, one or more regulatory elements (e.g., WPRE, PolyA, antibiotic resistance gene, etc.), and/or ITRs. Exemplary nucleic acid element combinations are provided in Table 1, and each combination of nucleic acid elements were incorporated into the respective plasmid vectors (Plasmids 1-30, see, also FIGS. 5A-5D) for assaying (e.g., for interferon delivery). A CAG promoter was used for the nucleic acids tested, and included a CMV intermediate early enhancer (CMVie) sequence, a chicken beta-actin sequence, and a rabbit beta-globin spice acceptor. CMVie sequences used were truncated and included either long (SEQ ID NO: 25), medium (SEQ ID NO: 26) or short (SEQ ID NO: 27) versions. Incorporation of a minigene, or “Xon” system, is further indicated (SEQ ID NOs: 31 and 51). A polynucleotide encoding human IFNβ that was either CpG depleted (SEQ ID NO: 4) or a representative wild-type (SEQ ID NO: 2) was used. A WPRE was used that was either modified (SEQ ID NO: 33) or a representative wild-type (SEQ ID NO: 32). Selected PolyA (SV40 as shown in SEQ ID NO: 19, and bGH as shown in SEQ ID NO: 34), antibiotic resistance gene (AmpR as shown in SEQ ID NO: 44 and KanR as shown in SEQ ID NOs: 20 or 21), and ITR sequence length are also shown (truncated ITR as shown in SEQ ID NOs: 40-41 and full length ITR as shown in SEQ ID NOs: 36-39).

TABLE 1
Exemplary Nucleic Acid Elements and Produced Plasmids

| | | | | Gene of | | | | |
|---|---|---|---|---|---|---|---|---|
| Plasmid | CMVie | CBA | Xon | Interest (GOI) | WPRE | PolyA | Resistance | ITR |
| #1 | Short | Short | No | hIFNβ-CpG | Mut6 | SV40 | AmpR | 130 |
| | | | | del | | | | |
| #2 | Short | Short | Yes | hIFNβ CpG | Mut6 | SV40 | AmpR | 130 |
| | | | | del w/o ATG | | | | |
| #3 | Long | Short | Yes | hIFNβ CpG | Mut6 | bGH | KanR | 130 |
| | | | | del w/o ATG | | | | |
| #4 | Long | Short | No | hIFNβ CpG | Mut6 | bGH | KanR | 130 |
| | | | | del | | | | |
| #5 | Long | Short | Yes | hIFNβ CpG | Mut6 | bGH | KanR | 145 |
| | | | | del w/o ATG | | | | |
| #6 | Long | Short | No | hIFNβ CpG | Mut6 | bGH | KanR | 145 |
| | | | | del | | | | |
| #7 | Short | Short | No | hIFNβ Wt (no | Mut6 | SV40 | AmpR | 130 |
| | | | | modification) | | | | |
| #8 | Long | Short | No | hIFNβ CpG | Mut6 | bGH | KanR | 130 |
| | | | | del | | | | |
| #9 | Long | Short | Yes | hIFNβ CpG | Mut6 | bGH | KanR | 130 |
| | | | | del w/o ATG | | | | |
| #10 | Long | Long | No | hIFNβ CpG | Mut6 | bGH | KanR | 130 |
| | | | | del | | | | |
| #11 | Long | Long | Yes | hIFNβ CpG | Mut6 | bGH | KanR | 130 |
| | | | | del w/o ATG | | | | |
| #12 | Long | Short | No | hIFNβ CpG | Mut6 | bGH | KanR- | 130 |
| | | | | del | | | CpGdel | |
| #13 | Long | Short | Yes | hIFNβ CpG | Mut6 | bGH | KanR- | 130 |
| | | | | del w/o ATG | | | CpGdel | |
| #14 | Long | Long | No | hIFNβ CpG | Mut6 | bGH | KanR- | 130 |
| | | | | del | | | CpGdel | |
| #15 | Long | Long | Yes | hIFNβ CpG | Mut6 | bGH | KanR- | 130 |
| | | | | del w/o ATG | | | CpGdel | |
| #16 | Long | Long | No | eGFP | Mut6 | bGH | KanR | 130 |
| #17 | Long | Long | Yes | eGFP w/o | Mut6 | bGH | KanR | 130 |
| | | | | ATG | | | | |
| #18 | Long | Short | No | eGFP | Mut6 | bGH | KanR- | 130 |
| | | | | | | | CpGdel | |
| #19 | Long | Short | Yes | eGFP w/o | Mut6 | bGH | KanR- | 130 |
| | | | | ATG | | | CpGdel | |
| #20 | Long | Short | No | mCardinal | Mut6 | bGH | KanR- | 130 |
| | | | | | | | CpGdel | |
| #21 | Long | Long | No | mCardinal | Mut6 | bGH | KanR- | 130 |
| | | | | | | | CpGdel | |
| #22 | Long | Short | Yes | mCardinal | Mut6 | bGH | KanR- | 130 |
| | | | | w/o ATG | | | CpGdel | |
| #23 | Long | Long | Yes | mCardinal | Mut6 | bGH | KanR- | 130 |
| | | | | w/o ATG | | | CpGdel | |
| #24 | Long | Short | No | hIFNβ_CpG | Mut6 | bGH | KanR- | 130 |
| | | | | del | | | CpGdel | |
| #25 | Long | Short | No | mCardinal | Mut6 | bGH | KanR- | 130 |
| | | | | | | | CpGdel | |
| #26 | Long | Short | No | Mouse IFNβ | Mut6 | bGH | KanR- | 130 |
| | | | | | | | CpGdel | |
| #27 | Long | Short | No | Rat IFNβ | Mut6 | bGH | KanR- | 130 |
| | | | | | | | CpGdel | |
| #28 | Long | Short | No | Canine IFNβ | Mut6 | bGH | KanR- | 130 |
| | | | | | | | CpGdel | |
| #29 | Long | Short | Yes | hIFNβ CpG | Mut6 | bGH | KanR- | 130 |
| | | | | del w/o ATG | | | CpGdel | |
| #30 | Long | Long | Yes | hIFNβ CpG | Mut6 | bGH | KanR- | 130 |
| | | | | del w/o ATG | | | CpGdel | |

### Materials and Methods

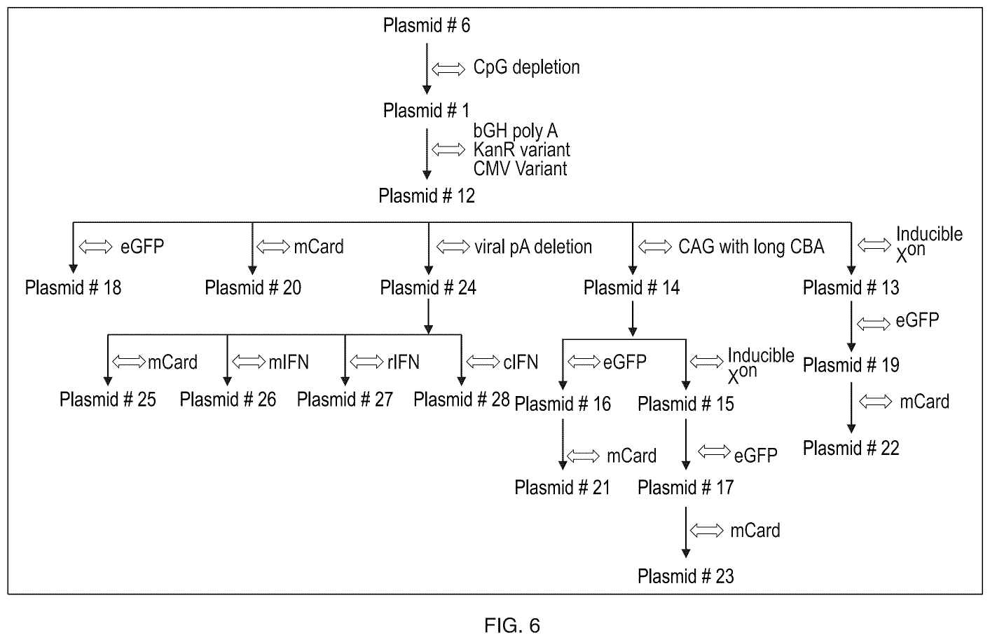

An exemplary workflow for producing engineered plasmids (by cloning) used to produce rAAV vectors provided by the present disclosure is shown in FIG. 6, which shows a multi-step cloning strategy to produce the plasmids. Exemplary protocols for producing certain engineered rAAV vectors are provided in the following.

To generate Plasmid #1 (FIG. 5A), a 582 bp neosynthesized polynucleotide of hIFNβ-CpGdel was subcloned into polynucleotide AAV-CAG-mCardinal by standard cloning methods, using KpnI and EcoRI restriction enzymes, in lieu of the mCardinal protein fluorescent marker ORF. To generate Plasmid #12 (FIG. 5L), two polynucleotide fragments were neosynthesized to cover both bGH poly A sequence (SEQ ID NO: 34) and KanR-CpG-depleted through CMV i.e. enhancer sequence (SEQ ID NO: 35) and inserted into Plasmid #1 using homologous recombination technique. Plasmid #14 (FIG. 5N) was created by inserted a 657 bp neosynthesized polynucleotide (SEQ ID NO: 54) encompassing into Plasmid #12. Plasmid #18 (FIG. 5R) and Plasmid #16 (FIG. 5P) were generated by replacing hIFNβ-CpG ORF from Plasmid #12 or Plasmid #14 respectively with a neosynthesized fragment for eGFP using BamHI/EcoRI. Plasmid #20 (FIG. 5T) and #21 (FIG. 5U) were obtained by replacing the hIFNβ-CpGdel ORF by a neosynthesized polynucleotide encoding for mCardinal in Plasmid #12 and #14, respectively. A neosynthesized polynucleotide fragment encompassing the splicing minigene (Xon) and a modified hIFNβ-CpGdel fragment, without ATG and without alternative start codons, was inserted into Plasmid #12 and Plasmid #14 to create Plasmid #13 (FIG. 5M) and Plasmid #15 (FIG. 5O), respectively. A modified neosynthesized eGFP fragment (without ATG, without alternative start codons), was inserted in lieu of hIFNβ-CpGdel in Plasmid #13 and Plasmid #15 to create Plasmid #17 (FIG. 5Q) and Plasmid #19 (FIG. 5S), respectively. A modified neosynthesized mCardinal fragment (without ATG, without alternative start codons), was inserted in lieu of hIFNβ-CpGdel in Plasmid #13 and Plasmid #15 to create Plasmid #22 (FIG. 5V) and Plasmid #23 (FIG. 5W), respectively.

### Example 2. Making Improved AAVs

This Example describes the making of improved AAVs with various combinations of regulatory elements for delivering a gene of interest to target cells.

Briefly, AAV stocks were generated using standard triple transfection protocols in combination with pRepCap and Adenovirus helper plasmids. The platform process used an adherent HEK293T triple transfection protocol, followed by a one-step purification using density gradient ultracentrifugation to remove empty AAV capsids. The sample was then concentrated and formulated in a 1-step process, using a spin column. These research grade lots were then formulated into an AAV-compatible excipient (1×dPBS/0.005% Poloxamer). The vector genomes (vg) titer for each lot was tested by RT qPCR with ITR primer sets within the AAV ITRs and results were provided on a Certificate of Analysis. AAV preparations #5, #6, #8 and #15 were produced with established GMP-like large-scale manufacturing suspension platforms (using suspension-adapted HEK293 cells). The AAVs that were produced are listed in Table 2.

TABLE 2
List of Recombinant AAV Research
Stocks Produced at Research Grade

| AAV | | Plasmid #used | Vg |
|---|---|---|---|
| preparation | AAV | for AAV | titer |
| (Prep) #* | description | production | (vg/mL) |
| 1 | AAV-CAG-hIFNβ-SV40 | Plasmid #7 | 3.13e13 |
| 2 | | | 2.19e13 |
| 3 | | | 3.59e13 |
| 4 | AAV-CAG-hIFNβ-SV40 | Plasmid #1 | 1.1e13 |
| 5 | | | 2.74e13 |
| 6 | | | 2.48e13 |
| 7 | | | 6.78e11 |
| 8 | | | 2.98e13 |
| 9 | | | 1.19e13 |
| 10 | AAV-CAG-hIFNβ-bGH | Plasmid #12 | 1.1e13 |
| 11 | AAV-CAG-hIFNβ-bGH | Plasmid #14 | 1.06e13 |
| 12 | AAV-CAG-mCard-bGH | Plasmid #20 | 3.04e13 |
| 13 | AAV-CAG-hIFNβ-bGH | Plasmid #24 | 1.71e13 |
| 14 | | | 1.79e13 |
| 15 | | | 2.80e13 |
| 16 | AAV-CAG-mCard-bGH | Plasmid #25 | 1.12e14 |
| 17 | AAV-CAG-mIFNβ-bGH | Plasmid #26 | 4.18e13 |
| 18 | AAV-CAG-X^{on}- | Plasmid #3 | 2.37e13 |
| | hIFNβ-CpGdel-KanR | | |

*AAV preparations are numbered arbitrarily, though the nomenclature is consistently used across experiments described in the Examples herein.
**The plasmid numbering is consistent with Table 1 and this nomenclature is consistently used across experiments described in the Examples herein.

These results confirmed that all AAVs were produced at high viral genome titers (>1e13 vg/mL), the suitability of the newly designed genomes for AAV packaging and transduction experiments, as well as robust and clinically relevant levels of viral production.

### Example 3. Validation of Various Variants of Therapeutic AAV Candidates

This Example describes the making of improved AA Vs for delivering a gene of interest to target cells and expression and activity of the payloads with various combinations of regulatory elements.

Variations within the payload and different regulatory elements were evaluated for their impact on the productivity and functionality of various candidates. In four constructs, the variables tested included: a CpG-deleted payload, two length variations of the CAG promoter, and variable poly A and antibiotic resistance cassettes. AAV preparations were tested side-by-side in a series of assays to quantify: 1) vector genome titers by digital PCR (dPCR) using payload primers; 2) capsid titers using a commercial ELISA kit; and 3) viral potency using an in vitro cell-based assay to measure the expression of the payload post-transduction in mammalian cells. The percentage of AAV full capsids and volumetric yields were calculated using the vg and capsid titers and data are shown in Table 3, represented as a relative ratio across the various production lots using AAV preparation #6 was used as an internal reference for all values tested concomitantly. For viral potency, the value obtained for each construct at MOI of 5e5 vg/cell was used to calculate the ratio across the production lots.

TABLE 3
Production of various AAV candidates at research scale

| | AAV | AAV | AAV | AAV |
|---|---|---|---|---|
| | prep- | prep- | prep- | prep- |
| | aration | aration | aration | aration |
| Parameter tested | #1 | #9 | #10 | #11 |
| Vg titer | 0.57 | 0.28 | 0.16 | 0.10 |
| Volumetric | 1.02 | 0.49 | 0.29 | 0.18 |
| yield (vg/L) | | | | |
| % AAV full | 1.89 | 0.70 | 1.80 | 1.48 |
| capsid | | | | |
| Potency | 1.72 | 1.46 | 2.23 | 1.90 |

Together, these results demonstrated that the various elements utilized in combination within the AAV genome plasmid did not significantly impact production or activity of the AAVs.

### Example 4. Large Scale AAV Batches for Assessing Clinical Manufacturing Suitability

This Example describes the making of large scale batches of improved AAVs with various combinations of regulatory elements for delivering a gene of interest to target cells.

Candidates with therapeutic payloads were produced using large scale suspension platforms. AAV batches were produced in both 2 L to 4 L scale harvests, fully purified, and assessed for various quality criteria. AAV batches were purified with multi-step processes to ensure high levels of purity in the final AAV materials. Steps included Tangential Flow Filtration (TFF), affinity capture chromatography, gradient ultracentrifugation or anion-exchange chromatography, and final formulation in a centrifugal device or another TFF. AAV stock genome titers were obtained using a PCR-based protocol. These AAV preparations were used to assess various critical quality attributes in side-by-side experiments.

Runs were conducted using Plasmid 1 (Table 4) or Plasmid 24 (Table 5) as exemplary representatives of AAVs described herein with a therapeutic payload. AAV preparations were tested side-by-side in a series of assays to quantify: 1) vector genome titers by digital PCR (dPCR) using primers located within the therapeutic payload; 2) capsid titers using a commercial ELISA kit; and 3) viral potency using an in vitro cell-based assay to measure the expression of the payload post-transduction in mammalian cells. The percentage of AAVs with full capsids, as well as volumetric yields, were calculated using the vg and capsid titers, and the data collected is shown in Table 4 and Table 5 represented as a relative ratio across the various production lots using an AAV preparation #5 (Table 4) or AAV preparation #6 (tested in parallel, except for capsids; Table 5) as a control reference, respectively. In the experiments presented in Table 4, for viral potency, the value obtained for each construct at MOI of 5e5 vg/cell was used to calculate the ratio across the production lots. In the experiments presented in Table 5, for viral potency, the value obtained for each construct at MOI of 3.9e5 vg/cell was used to calculate the ratio across the production lots.

TABLE 4
Runs Using Plasmid #1

| | Run 2 (AAV | Run 3 (AAV |
|---|---|---|
| Parameter tested | preparation 6) | preparation 8) |
| Vg titer | 1.11 | 1.57 |
| Total yield (vg) | 2.23 | 4.79 |
| Volumetric | 1.11 | 1.28 |
| yield (vg/L) | | |
| Capsid titer | 1.85 | 2.44 |
| % AAV full | 0.60 | 0.65 |
| capsid | | |
| Potency | 0.59 | 1.34 |

TABLE 5
Runs Using Plasmid #24

| | Run 4 (AAV |
|---|---|
| Parameter tested | preparation 15) |
| Vg titer | 1.49 |
| Total yield (vg) | 0.85 |
| Volumetric yield (vg/L) | 0.43 |
| % AAV full capsid | 1.33 |
| Potency | 3.45 |

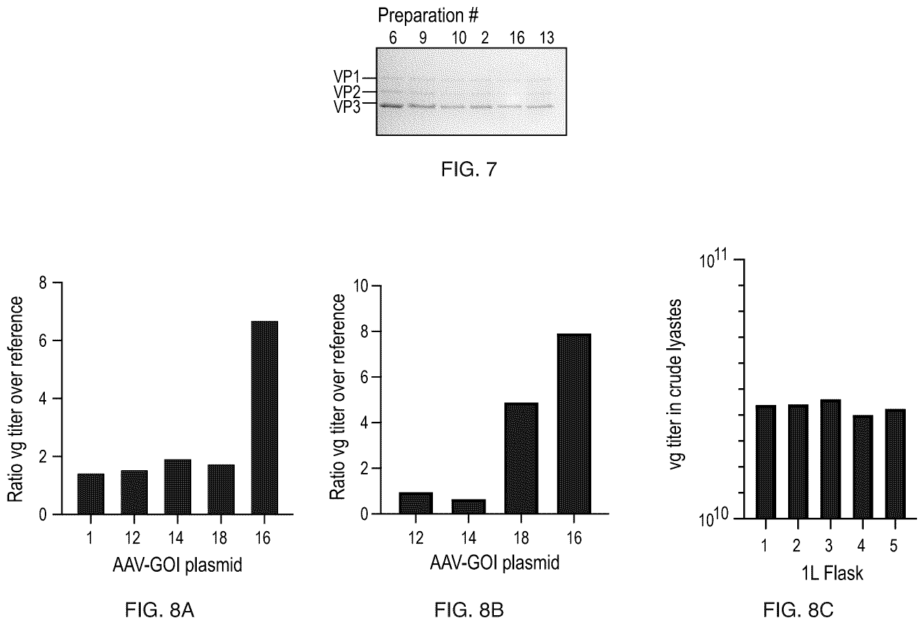

The purity and the presence of AAV capsid proteins at the expected ratios were confirmed in several AAV preparations. Briefly, 1e11 total vg of each preparation was assessed by gel electrophoresis followed by Coomassie blue staining in denaturing conditions (FIG. 7). For each preparation, 3 capsid proteins (VP1, VP2 and VP3) were identified at the expected ratio of approximately 1:1:10, respectively, and no other proteins were detected, thereby confirming a high level of purity and an absence of detectable impurities or contaminants.

These results demonstrated an orthogonal validation of therapeutic AAV candidates' production performance (each produced at a different manufacturing vendor), highlighting the usability across facilities and robust production processes across users and different processes. All subtle variability observed between different production service providers were within the expected ranges for small batch productions.

Taken together, these successful cloning experiments and viral productions demonstrated that high viral genome titers were obtained (>1e13 vector genomes (vg)/mL) at a high level of purity and homogeneity for each AAV construct, as well as the suitability of the newly designed genomes for AAV packaging and transduction experiments and robust and clinically relevant levels of viral production.

### Example 5: Side-By-Side Production of Improved AAV Variants for Validation of AAV Vectors with Therapeutic Payloads

This Example describes a comparison of functionality and packaging of different AAV vectors with therapeutic payloads.

Briefly, HEK293 cells grown in suspension (VPC2.0 cells, ThermoFisher Scientific) were transfected with each of the pAAV plasmids individually, together with pRepCap and pAd helper plasmids to support the packaging of the AAV genomes. Upon production, cells were lysed, and submitted to nuclease digestion (Benzonase) and the crude lysates were clarified by centrifugation to remove cell membranes and debris. Virus preparations were stored frozen until testing of the vg titer using a digital dPCR protocol with primers and probe sets specific to the respective payloads (FIGS. 8A-8C).

Taken together, these experiments demonstrated that successful viral productions were produced with titers obtained within the expected range (>1e10 vg/mL) for each AAV construct, as well as the suitability of the newly-designed genomes for AAV packaging and transduction experiments and clinically relevant levels of viral production.

### Example 6: Demonstration of Potency of AAV with Therapeutic Payloads

This Example describes the potency of payloads delivered with AAVs described herein.

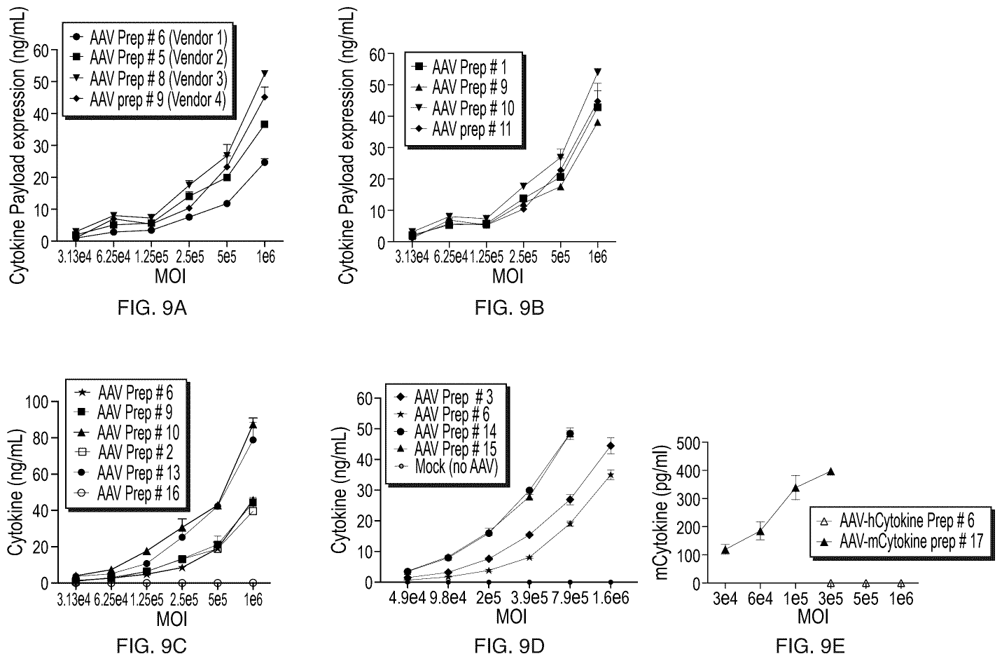

Briefly, HEK293T cells were plated in 96-well plates and transduced at a multiplicity of infection ranging from approximately 3e4 to 1e6 vg/cell with several variants of AAV-CAG-hIFNβ. 48 hours post-transduction with AAV, media supernatants were harvested for payload measurement. The amount of hIFNβ was measured in nanograms per milliliter (ng/ml; FIGS. 9A-9D) or picograms per milliliter (pg/mL; FIG. 9E) using a commercial ELISA kit for human (h) IFNβ (FIG. 9A-D) or mouse (m) IFNβ (FIG. 9E).

In particular, in the first set of experiments, all preparations were produced from Plasmid #1, carrying a hIFNβ payload, and packaged within the same AAV capsid serotype, yet produced by four different vendors (FIG. 9A). In analyzing the results, it was observed that there was a lower potency when produced at a specific vendor (AAV preparation #6). However, as shown earlier, no other differences were detected. All other preparations (#5, #8, and #9) generated from Plasmid 1 demonstrated similar potency as measured by payload expression.

In a set of experiments, Plasmid #7 (corresponding to AAV prep #1 in FIG. 9B or AAV prep #3 in FIG. 9D), Plasmid #1 (corresponding to AAV prep #9 in FIG. 9B or AAV prep #6 in FIG. 9D), Plasmid #12 (corresponding to AAV prep #10 in FIG. 9B) and Plasmid #14 (corresponding to AAV prep #11 in FIG. 9B), all produced at one vendor, and packaged within the same AAV capsid serotype, demonstrated no significant difference in potency across multiple variants of our AAV constructs and payloads, except for preparation #6 as shown in FIG. 9A.

AAVs carrying a payload other than IFN did not produce measurable IFN expression, as expected. It was also demonstrated that AAV preparation #15 showed one of the highest levels of potency across the constructs and vendors (AAV preparation #15 was prepared with processes similar to those for use in clinical batch production).

In a set of experiments, human (h) and mouse (m) cytokine payloads were compared, and it was demonstrated that the mouse cytokine was able to be expressed in human cells (FIG. 9E), thereby demonstrating that such a payload can be used as a control in various in vitro and in vivo models.

Taken together, these results demonstrated that AAVs described herein could successfully deliver a functional therapeutic payload e.g., hIFNβ or mIFNβ, into mammalian cells.

### Example 7: Demonstration of Payload Activity Upon Delivery in Mammalian Cells

This Example describes the activity of payloads delivered with AAVs described herein.

An activity assay using a reporter cell line was used for the measurement of therapeutic transgene activity. This method utilized a reporter cell line of human promonocytic cells (U937) engineered to express Firefly Luciferase under the control of an IFN α/β responsive promoter. In this model, when IFNα or IFNβ binds to the IFNα/β receptor (IFNAR1) on the cell surface, the IFNα/β-regulated Firefly Luciferase reporter gene construct will be activated, resulting in a luminescent signal that was precisely quantified.

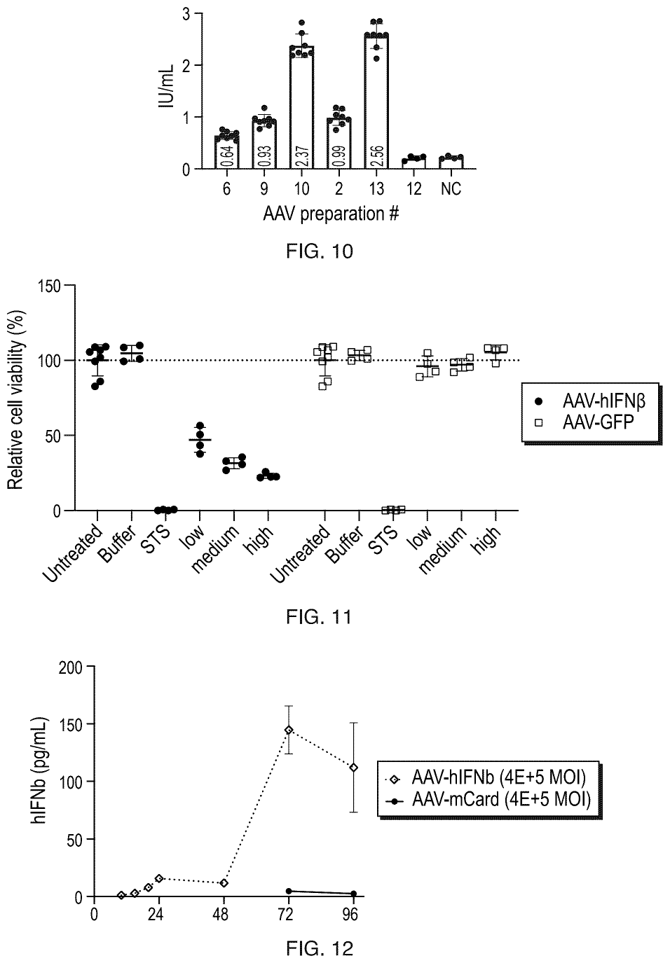

Briefly, AAV preparations were incubated with reporter cells at a MOI of 5e5 vg/cell. AAV preparation #12 (expressing a reporter payload mCardinal) and cell culture medium (NC) were used as a negative controls. Measurements were obtained after an 18-hour incubation period at 37° C. and 5% CO₂. The standard curve was generated using a commercial recombinant hIFNβ1 cytokine (Prospect CYT-234) and expressed in infectious units/mL. Luciferase detection and measurement were obtained using a reporter detection assay (FIG. 10). These results demonstrated that various hIFNβ payloads successfully expressed and were biologically active in target cells. The data also correlated with potency measurements via payload expression shown in FIGS. 9A-9E, with preparations #10 and #13 showing the highest activity and highest expression (FIG. 9C) and preparation #6 having the lowest activity and expression (FIGS. 9A and 9D).

Taken together, these data demonstrated the functionality of vectors described herein, which successfully delivered a biologically active hIFNβ upon transduction in human cells in an in vitro model.

### Example 8: Demonstration of Therapeutic Activity of AAV with Therapeutic Payloads in Cancer Cells

This Example describes the therapeutic activity of payloads delivered to cancer cells (e.g., brain cancer cells e.g., brain cancer cells, wherein the cancer metastasized from a primary cancer originating in a different location in the body) with AAVs described herein.

A patient-derived xenograft organoid (PDXO) model was established from a secondary breast cancer metastasis in the brain and the resulting organoids were used to monitor the anti-tumor efficacy of AAV with a therapeutic payload. Organoids were established using a proprietary method by a qualified manufacturing vendor. Organoids were exposed to three doses of AAV preparation #6 (expressing hIFNβ), as well as an AAV negative control expressing GFP (AAV-GFP) for 2 to 4 hours in a small volume of media before being seeded in a hydrogel. Untreated organoids were exposed to media only. In addition to AAV dosing, replicate wells were included which received 1 μm Staurosporin as a positive killing control or the AAV formulation buffer as a negative, killing control. At 6 days post-treatment, replicate wells (n=4) of treatment conditions were assessed for cell viability using the reporter assay (Promega G7570) for measuring total viable cells through the generation of a luminescent signal expressed as luminous flux (lux) that is directly proportional to the amount of ATP, and thus cells, present in a culture. Organoids from the technical replicate wells per treatment condition were additionally collected, fixed, washed in PBS, and stained with Hoechst and Rhodamine Phalloidin to stain nuclei and eukaryotic cytoskeleton, respectively. Stained organoids were imaged at 4× magnification and subsequently processed for high content imaging analysis in the 3D analysis platform Ominer® to quantify the compound induced effects on tumor outgrowth.

As shown in FIG. 11, a dose-dependent decrease in organoid viability was observed upon infection with AAV-hIFNβ but not AAV-GFP at 6 days post-infection. In groups treated with AAV-hIFNβ, a time-dependent reduction in cell viability was observed at all virus concentrations tested. It can be noted that when treated with AAV-GFP, no notable changes in any high content imaging features were observed 6 days post-treatment, indicating that treatment with AAV-GFP was not therapeutic in this model at the doses tested. A dose- and time-dependent increase in the fraction of dead cells was observed in organoids treated with AAV-hIFNβ, and a strong reduction in total organoid size and the number of nuclei per organoid was also observed at all dose levels of AAV-hIFNβ. Treatment with AAV-IFNβ showed a small reduction in organoid and nuclei count, and total nuclei size, compared to untreated and buffer-treated organoids at the highest virus dose tested.

These results demonstrated that hIFNβ payloads were successfully expressed and biologically active in target cancer cells (e.g., target brain cancer cells where the cancer metastasized from a primary cancer elsewhere in the body of a subject) and therapeutically efficacious for the treatment of cancer. Together, these results indicated that AAV-IFNβ elicited an anti-tumor effect that was primarily cytotoxic, particularly at the highest tested dose level. The data demonstrated functionality of AAV-hIFNβ to elicit anti-neoplastic effects on human tumor cells in an ex vivo model of brain metastasis.

### Example 9. Extended AAV-Delivered Payload Expression in GBM Cancer Cells

This Example describes the therapeutic activity of payloads delivered to brain cancer cells with AAVs described herein.

In this Example, immortalized glioblastoma (GBM) cells were exposed either to AAVs described herein expressing a hIFNβ payload or a recombinant hIFNβ cytokine (not vectorized). Following exposure, hIFNβ levels were monitored in the cell media for a period of 4 days.

Briefly, cells were plated in 48-well plates and exposed to a defined MOI of AAV preparation #13 (hIFNβ, 4e5 vg/cell) or AAV preparation #12 (mCardinal, 4e5 vg/cell). Full media exchanges were performed daily for a total of 4 days. Negative control wells were exposed to an AAV expressing the control, a fluorescent protein mCardinal (preparation #12), and media without an active agent. hIFNβ levels were measured from collected supernatant using a hIFNβ High Sensitivity ELISA kit (PBL Cat 41415).

Secreted hIFNβ levels were quantifiable as early as 10 hours post-transduction, with peak expression observed after 72 hours and maintained to 96 hours. AAV transduction, alone, did not stimulate therapeutic levels of hIFNb secretion from GBM cells in vitro, as evidenced by the low levels of hIFNβ from AAV-mCardinal-transduced cells. Indeed, supernatants from AAV-mCardinal-treated cells were below the assay limit of quantitation in 4 out of 4 samples at 24 hours, 4 out of 4 samples at 48 hours, 2 out of 4 samples at 72 hours, and 3 out of 4 samples at 96 hours post-treatment. Meanwhile, of the 3 replicates described in this experiment with detectable levels (plotted in FIG. 12), none exceeded 5 μg/mL.

Taken together, the results demonstrated that AAVs described herein can successfully transduce human GBM cells to deliver a sustained hIFNβ payload.

### Example 10. Production and Testing of Therapeutic AAVs at Clinical Grade to Initiate Phase I/II Clinical Trial in Brain Cancer Patients

This Example describes the making of clinical grade AAVs with various combinations of regulatory elements for delivering a gene of interest.

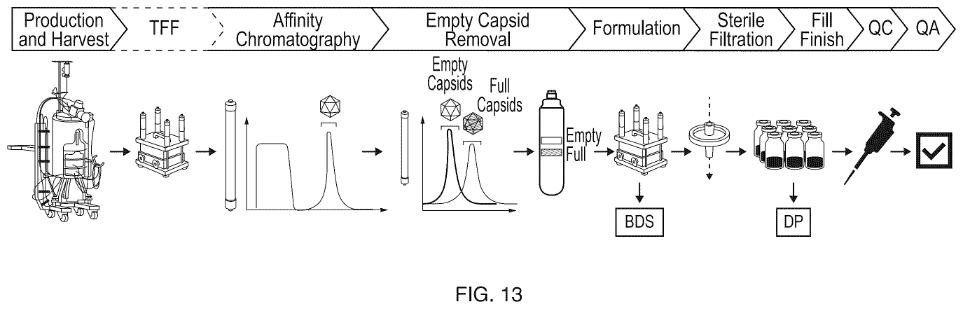

An AAV containing a therapeutic payload is produced in compliance with Current Good Manufacturing Practice (cGMP) regulations in plasmid batches. Plasmid batches are used to produce viral drug product. Exemplary cGMP-compliant manufacturing processes are shown in FIG. 13.

To generate cGMP-compliant plasmid batches, a Master Cell Bank (MCB) is generated for plasmids described herein, such as, for example, Plasmid #24 having a long CMVie, short CBA, CpG-deleted hINFβ payload, mutant WPRE, bGH polyA, and CpG-deleted Kanamycin resistance gene. Furthermore an E. coli strain is selected to produce plasmids at a high quality, high homogeneity, high stability, and majority supercoiled confirmation to enable optimal AAV production by transfection. Plasmids generated are used to produce various AAV batches at Good Laboratory Practice (GLP) and Good Manufacturing Practice (GMP) grades to support preclinical development, including, but not limited, to IND-enabling toxicology studies and clinical trials.

A series of highly-specific assays are developed to test the AAV GLP- and GMP-compliant batches. These assays are used to assess AAV drug product for safety, strength, potency, and purity criteria, as well as any other suitable critical quality attributes.

What is claimed is:

1. A recombinant adeno-associated viral (rAAV) vector comprising, from 5′ to 3′:
a) a first inverted terminal repeat (ITR) sequence at least 95% identical to the sequence as set forth in SEQ ID NO: 40;
b) a CAG promoter sequence at least 95% identical to the sequence as set forth in SEQ ID NO: 48;
c) a polynucleotide sequence at least 95% identical to the sequence set forth in SEQ ID NO: 4 encoding a CpG-depleted human interferon β (IFNβ);
d) a Woodchuck Hepatitis Virus (WHP) Posttranscriptional Regulatory Element (WPRE) sequence at least 95% identical to the sequence as set forth in SEQ ID NO: 33;
e) a bovine growth hormone (bGH) polyadenylation (poly) A signal sequence least 95% identical to the sequence as set forth in SEQ ID NO: 34; and
f) a second ITR sequence at least 95% identical to the sequence as set forth in SEQ ID NO: 40.

2. The rAAV vector of claim 1, comprising:
a) the first ITR sequence as set forth in SEQ ID NO: 40;
b) the CAG promoter sequence as set forth in SEQ ID NO: 48;
c) the polynucleotide sequence as set forth in SEQ ID NO: 4 encoding the CpG-depleted human interferon β (IFNβ);
d) the WPRE sequence as set forth in SEQ ID NO: 33;
e) the bGH poly A signal sequence as set forth in SEQ ID NO: 34; and
f) the second ITR sequence as set forth in SEQ ID NO: 40.

3. The rAAV vector of claim 1, wherein the human IFNβ comprises an amino acid sequence as set forth in SEQ ID NO: 1.

4. The rAAV vector of claim 1, wherein the polynucleotide sequence encoding the CpG-depleted human IFNβ comprises reduced CpG dinucleotides and/or increased methylation of CpG dinucleotides as compared to a parental equivalent.

5. The rAAV vector of claim 4, wherein methylation of the CpG dinucleotides is increased by at least about 50% as compared to a parental equivalent.

6. The rAAV vector of claim 5, wherein the CpG dinucleotides are completely methylated.

7. The rAAV vector of claim 4, wherein the CpG dinucleotides are reduced by at least about 50% as compared to a parental equivalent.

8. The rAAV vector of claim 7, wherein the CpG dinucleotides are depleted.

9. The rAAV vector of claim 1, wherein the rAAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof.

10. The rAAV vector of claim 1, wherein the rAAV is AAV9 or a derivative thereof.

11. The rAAV vector of claim 1, further comprising an antibiotic resistance gene.

12. The rAAV vector of claim 11, wherein the antibiotic resistance gene confers resistance to kanamycin, spectinomycin, streptomycin, ampicillin, carbenicillin, bleomycin, erythromycin, polymyxin B, tetracycline, chloramphenicol, neomycin, zeocin, or a derivative thereof.

13. The rAAV vector of claim 12, wherein the antibiotic resistance gene confers resistance to kanamycin and comprises a nucleic acid sequence at least about 90% identical to the sequence of SEQ ID NO: 21.

14. The rAAV vector of claim 13, wherein the antibiotic resistance gene comprises reduced CpG dinucleotides and/or increased methylation of CpG dinucleotides as compared to a parental equivalent.

15. A method of treating cancer in a subject in need thereof comprising administering the rAAV vector of claim 1.

16. The method of claim 15, wherein the cancer is glioblastoma, metastatic brain tumor, or uveal melanoma.

17. The method of claim 15, wherein the administration is to the central nervous system.

18. The method of claim 15, wherein the administration is to the brain.

19. The method of claim 15, wherein the administration is by Convection Enhanced Delivery (CED).

20. The method of claim 15, wherein the administration is by intratumoral injection, intracranial injection, intracerebral injection, intracerebroventricular, intraparenchymal, or injection into the cerebrospinal fluid (CSF) via the cerebral ventricular system, cisterna magna, or intrathecal space.


SEQUENCE LISTING (72 sequences)

SEQ ID NO: 1 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 2 - DNA, 564 bp, Homo sapiens
  source 1..564: mol_type = unassigned DNA
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactaa

SEQ ID NO: 3 - DNA, 564 bp, synthetic construct
  source 1..564: mol_type = other DNA
atgactaataagtgcctgctgcagatagccctgctcctgtgttttagcaccacggcactgagtatgtcctataatctgctgggctttctgcagaggtcatcaaacttccaatgccaaaagttgctctggcagctgaacggccggctggagtattgtctgaaagatcggatgaatttcgatattcccgaagaaatcaaacaattgcaacaatttcaaaaggaagatgcagccctgacgatttacgaaatgctgcaaaatattttcgcaatctttaggcaggacagcagctccacaggttggaacgaaaccatagtggaaaatctgctcgcgaacgtgtaccaccaaatcaatcacttgaaaaccgtgctcgaggagaagttggaaaaggaagactttacacgtggcaagcttatgtcttccttgcatctcaagaggtactacggccggatactccactatcttaaagctaaagaatattcccattgcgcgtggacaatcgtgcgcgttgagattctgcgcaatttctattttatcaatcgactgacgggctatttgcggaattga

SEQ ID NO: 4 - DNA, 564 bp, synthetic construct
  source 1..564: mol_type = other DNA
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggatgctgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctcagaaactaa

SEQ ID NO: 5 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 6 - DNA, 570 bp, Homo sapiens
  source 1..570: mol_type = unassigned DNA
atggcctcgccctttgctttactgatggtcctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataa

SEQ ID NO: 7 - DNA, 570 bp, synthetic construct
  source 1..570: mol_type = other DNA
atggcttctcctttcgcccttctcatggtactcgttgttttgtcctgtaaaagctcctgtagccttggatgcgaccttccggaaacacatagtcttgacaatcgaagaacactgatgttgcttgctcagatgtcacgcattagccccagcagttgccttatggatcgccacgatttcgggttcccgcaagaagaattcgacgggaatcaatttcagaaagcgcctgctatttcagtgctgcacgaattgattcaacaaatatttaatctgttcacaaccaaggacagttccgccgcctgggacgaagatcttctggataagttttgtacggagttgtatcaacaattgaacgatctggaagcttgcgtcatgcaagaagaacgcgtaggcgagaccccactcatgaacgcagattctattctggcagttaagaagtattttaggcggataacgctgtacttgactgaaaagaagtatagtccctgcgcttgggaagtggtgcgggccgagataatgcggtctcttagcctctctaccaatctgcaggagaggctgcgccgtaaagagtag

SEQ ID NO: 8 - DNA, 570 bp, synthetic construct
  source 1..570: mol_type = other DNA
atggcctcaccctttgctttactgatggtcctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcactgaactctaccagcagctgaatgacttggaagcctgtgtgatgcaggaggagagggtgggagaaactcccctgatgaatgctgactccatcttggctgtgaagaaatacttcagaagaatcactctctatctgacagagaagaaatacagcccttgtgcctgggaggttgtcagagcagaaatcatgagatccctctctttatcaacaaacttgcaagaaagattaaggaggaaggaataa

SEQ ID NO: 9 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 10 - DNA, 501 bp, Homo sapiens
  source 1..501: mol_type = unassigned DNA
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 11 - DNA, 501 bp, synthetic construct
  source 1..501: mol_type = other DNA
atgaagtacacctcttacatacttgccttccaactgtgtatagtcctgggaagcctggggtgctattgtcaagatccctacgtcaaggaagctgagaatctgaagaagtacttcaacgccggccactccgacgttgccgacaacggcacgctgtttctgggtatcctgaagaactggaaggaagaatctgatcgaaagatcatgcaatctcagatcgtgtctttctatttcaagctcttcaagaacttcaaggacgaccaatcaattcagaaaagcgtggaaacgataaaagaggatatgaacgttaaattctttaactccaacaagaagaagcgcgacgatttcgagaaattgacgaactacagcgttaccgatctcaacgtgcagagaaaggccatccacgagctgattcaggtaatggccgagctctcacctgcggccaagaccggcaaaagaaagcggagccaaatgcttttcaggggcagacgggcttcacaatga

SEQ ID NO: 12 - DNA, 501 bp, synthetic construct
  source 1..501: mol_type = other DNA
atgaaatatacaagttatatcttggcttttcagctctgcatagttttgggttctcttggctgttactgccaggacccatatgtaaaagaagcagaaaaccttaagaaatattttaatgcaggtcattcagatgtagctgataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaaagggatgactttgaaaagctgactaattattcagtaactgacttgaatgtccaaaggaaagcaatacatgaactcatccaagtgatggctgaactgtcaccagcagctaaaacagggaagaggaaaaggagtcagatgctgtttagaggtaggagagcatcccagtaa

SEQ ID NO: 13 - protein, 182 aa, Mus musculus
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 14 - DNA, 549 bp, Mus musculus
  source 1..549: mol_type = unassigned DNA
atgaacaacaggtggatcctccacgctgcgttcctgctgtgcttctccaccacagccctctccatcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaactaa

SEQ ID NO: 15 - protein, 186 aa, unidentified
  source 1..186: note = Canis lupus familiaris
MTSRCILQTTLLLYFSTMALAMSNDLLRSQLSSSSLECQELLLQLNGTTEYCLKDRINFEIPEEIEKSRQFQKEDIILITHEMFQKIFDIFRRNISRTGWNETTVENLLVKLHWQKEHLEIILEDVKEKENFTWDNRTLLHLKKYYLRIVQYLKAKEYSICAWTIVQAEICRNFFFLNILTDYLQN

SEQ ID NO: 16 - DNA, 561 bp, unidentified
  source 1..561: mol_type = unassigned DNA; note = Canis lupus familiaris
atgaccagtagatgcatcctccaaacaactctcctgttgtatttctccaccatggctcttgccatgagcaacgacttgcttcgatcccagctaagcagcagcagtttggagtgtcaggagctcctattacagttgaatggaaccactgaatattgcctcaaggacaggataaacttcgagatccctgaggaaatcgagaaatcacgccagttccagaaggaggacatcatattgatcacccatgagatgttccagaagatctttgatattttcaggagaaatatctctagaacaggatggaatgagaccactgtcgagaaccttcttgtgaagctccactggcagaaggaacatctggagataatcctggaggacgtcaaagagaaggaaaacttcacctgggacaacaggactcttctgcacctgaagaaatattacttaaggattgtgcagtacctgaaggccaaggagtacagcatctgtgcctggacaatagtccaagcagaaatctgcaggaactttttcttccttaatatacttacagattatctccagaactga

SEQ ID NO: 17 - protein, 19 aa, porcine teschovirus-1
ATNFSLLKQAGDVEENPGP

SEQ ID NO: 18 - protein, 18 aa, thosea asigna virus
EGRGSLLTCGDVEENPGP

SEQ ID NO: 19 - DNA, 122 bp, Simian virus 40
  source 1..122: mol_type = unassigned DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 20 - DNA, 795 bp, synthetic construct
  source 1..795: mol_type = other DNA
tcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcat

SEQ ID NO: 21 - DNA, 795 bp, synthetic construct
  source 1..795: mol_type = other DNA
tcagaagaactcatcaagaagtctatagaaggcaattctctgggaatcaggagctgcaataccataaagcacaaggaatctgtcagcccattcaccaccaagctcttcagcaatatctctggtagccaaggctatgtcctgatatctgtctgccacacccagtctgccacagtcaatgaatccagaaaatctgccattttccaccatgatatttggcaagcaggcatcaccatgggtcacaacaagatcctcaccatctggcatgctggccttgagcctggcaaacagttctgctggggcaagcccctgatgctcttcatccagatcatcctgatcaacaagacctgcttccattctagttcttgctctctcaattctatgtttggcttggtggtcaaatgggcaggtagctggatcaagggtatgcagtcttctcattgcatcagccatgatggatactttctctgcaggagcaaggtgagatgacaggagatcctgccctggcacttcacccaatagcagccagtcccttcctgcttcagtgacaacatcaagcacagctgcacaaggaacaccagtggtggccagccaggatagtctggctgcctcatcttgcagttcattcagggcacctgacaggtcagtcttgacaaaaagaactggtctcccctgggctgacagcctgaacactgctgcatcagagcagccaattgtctgttgtgcccagtcatagccaaatagcctctccacccaagctgctggagaacctgcatgcaatccatcttgttcaatcat

SEQ ID NO: 22 - DNA, 584 bp, synthetic construct
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 23 - DNA, 933 bp, synthetic construct
  source 1..933: mol_type = other DNA
ttgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 24 - skipped

SEQ ID NO: 25 - DNA, 367 bp, Human cytomegalovirus
  source 1..367: mol_type = unassigned DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggt

SEQ ID NO: 26 - DNA, 304 bp, synthetic construct
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 27 - DNA, 246 bp, synthetic construct
  source 1..246: mol_type = other DNA
ttgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 28 - DNA, 278 bp, synthetic construct
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 29 - DNA, 315 bp, synthetic construct
  source 1..315: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagc

SEQ ID NO: 30 - DNA, 41 bp, Oryctolagus cuniculus
  source 1..41: mol_type = unassigned DNA
cctctgctaaccatgttcatgccttcttctttttcctacag

SEQ ID NO: 31 - DNA, 1170 bp, synthetic construct
  source 1..1170: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgtggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttgagtcaatccaagtgccaccatgcaggaggttcatgattgtgtagagtaagacataattttgttgaggtttaactctgaatacttaatgtggtactgaatacttaatgtggtactgagaggcagcctaactgaccacacagcattcatatttcatgttgttattttctctgatcctcattatggctcttcctgcgggtttgagtcttcatttgggtaatttagttattctttgctgcatttgattaaggaaatacgtatatgggaatttggagatttgtgactgacttcactatgtctaatttttttttgagacggagttttgctctgttgcccaggccggagtgcagtggcacagtcttggctcacagcaacctctacctcctgggttcaagcgattctcctgccttagcctcccgagtagctgggattacaggcatgtgccaccacgcctggctaatttttgtatttttagtagagacggggtttcaccatggtggccaggctgggttctggttgtttatgatctttattttttggtgatctaggaaccaaacaacaagaaattgttgtttcccgtgggtaagaggatcc

SEQ ID NO: 32 - DNA, 589 bp, Woodchuck hepatitis virus
  source 1..589: mol_type = unassigned DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 33 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 34 - DNA, 208 bp, Bos sp.
  source 1..208: mol_type = unassigned DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 35 - DNA, 1753 bp, synthetic construct
  source 1..1753: mol_type = other DNA
cagaagaactcatcaagaagtctatagaaggcaattctctgggaatcaggagctgcaataccataaagcacaaggaatctgtcagcccattcaccaccaagctcttcagcaatatctctggtagccaaggctatgtcctgatatctgtctgccacacccagtctgccacagtcaatgaatccagaaaatctgccattttccaccatgatatttggcaagcaggcatcaccatgggtcacaacaagatcctcaccatctggcatgctggccttgagcctggcaaacagttctgctggggcaagcccctgatgctcttcatccagatcatcctgatcaacaagacctgcttccattctagttcttgctctctcaattctatgtttggcttggtggtcaaatgggcaggtagctggatcaagggtatgcagtcttctcattgcatcagccatgatggatactttctctgcaggagcaaggtgagatgacaggagatcctgccctggcacttcacccaatagcagccagtcccttcctgcttcagtgacaacatcaagcacagctgcacaaggaacaccagtggtggccagccaggatagtctggctgcctcatcttgcagttcattcagggcacctgacaggtcagtcttgacaaaaagaactggtctcccctgggctgacagcctgaacactgctgcatcagagcagccaattgtctgttgtgcccagtcatagccaaatagcctctccacccaagctgctggagaacctgcatgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtacctagttattaatagtaatcaattacggggtcattagttcatagcccatatatggagttccgcgttacataacttacggtaaatggcccgcctggctgaccgcccaacgacccccgc

SEQ ID NO: 36 - DNA, 145 bp, Adeno-associated virus
  source 1..145: mol_type = unassigned DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 37 - DNA, 145 bp, Adeno-associated virus
  source 1..145: mol_type = unassigned DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 38 - DNA, 145 bp, Adeno-associated virus
  source 1..145: mol_type = unassigned DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 39 - DNA, 145 bp, Adeno-associated virus
  source 1..145: mol_type = unassigned DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 40 - DNA, 130 bp, Adeno-associated virus
  source 1..130: mol_type = unassigned DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 41 - DNA, 130 bp, Adeno-associated virus
  source 1..130: mol_type = unassigned DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 42 - DNA, 549 bp, synthetic construct
  source 1..549: mol_type = other DNA
atgaacaacaggtggatcctccatgctgcattcctgctgtgcttctccaccacagccctctccatcaactataagcagctccagctccaagaaaggacgaacattcggaaatgtcaggagctcctggagcagctgaatggaaagatcaacctcacctacagggctgacttcaagatccctatggagatgactgagaagatgcagaagagttacactgcctttgccatccaagagatgctccagaatgtctttcttgtcttcagaaacaatttctccagcactgggtggaatgagactattgttgtaaggctcctggatgaactccaccagcagacagtgtttctgaagacagtactagaggaaaagcaagaggaaagattgacctgggagatgtcctcaactgctctccacttgaagagctattactggagggtgcaaagataccttaaactcatgaagtacaacagctatgcctggatggtggtcagggcagagatcttcaggaactttctcatcattaggagacttaccagaaacttccaaaactaa

SEQ ID NO: 43 - DNA, 819 bp, unidentified
  source 1..819: mol_type = unassigned DNA; note = Canis lupus familiaris
tgggaaattcctctgcgatagaaagaggaccatcctgtataaatagggctacactcctggtagaagggcatttgcgctctgaactctcagagctttggcttcagcatcgagatggtaataggtgagactcttcccattttcatcatgaccagtagatgcatcctccaaacaactctcctgttgtatttctccaccatggctcttgccatgagcaacgacttgcttcgatcccagctaagcagcagcagtttggagtgtcaggagctcctattacagttgaatggaaccactgaatattgcctcaaggacaggataaacttcgagatccctgaggaaatcgagaaatcacgccagttccagaaggaggacatcatattgatcacccatgagatgttccagaagatctttgatattttcaggagaaatatctctagaacaggatggaatgagaccactgtcgagaaccttcttgtgaagctccactggcagaaggaacatctggagataatcctggaggacgtcaaagagaaggaaaacttcacctgggacaacaggactcttctgcacctgaagaaatattacttaaggattgtgcagtacctgaaggccaaggagtacagcatctgtgcctggacaatagtccaagcagaaatctgcaggaactttttcttccttaatatacttacagattatctccagaactgaagatctctcagcctctgcctctcggaatggacgatgcttgacaaggatttcaggcattcctcaactggcagatgctctgtaagtgacagctaatgtactgcatttgaaaggaca

SEQ ID NO: 44 - DNA, 861 bp, synthetic construct
  source 1..861: mol_type = other DNA
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 45 - DNA, 263 bp, synthetic construct
  source 1..263: mol_type = other DNA
ggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgc

SEQ ID NO: 46 - DNA, 105 bp, synthetic construct
  source 1..105: mol_type = other DNA
actcttcctttttcaatattattgaagcatttatcagggttattgtctcatgagcggatacatatttgaatgtatttagaaaaataaacaaataggggttccgcg

SEQ ID NO: 47 - DNA, 103 bp, synthetic construct
  source 1..103: mol_type = other DNA
tttagcttccttagctcctgaaaatctcgataactcaaaaaatacgcccggtagtgatcttatttcattatggtgaaagttggaacctcttacgtgccgatca

SEQ ID NO: 48 - DNA, 991 bp, synthetic construct
  source 1..991: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 49 - DNA, 1720 bp, synthetic construct
  source 1..1720: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaatt

SEQ ID NO: 50 - DNA, 1648 bp, synthetic construct
  source 1..1648: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 51 - DNA, 572 bp, synthetic construct
  source 1..572: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttgagtcaatccaagtgccaccatgcaggaggttcatgattgtgtagagtaagacataattttgttgaggtttaactctgaatacttaatgtggtactgaatacttaatgtggtactgagaggcagcctaactgaccacacagcattcacgcctggctaatttttgtatttttagtagagacggggtttcaccatggtggccaggctgggttctggttgtttatgatctttattttttggtgatctaggaaccaaacaacaagaaattgttgtttcccgtgggtaagaggatcc

SEQ ID NO: 52 - DNA, 958 bp, Gallus gallus
  source 1..958: mol_type = unassigned DNA
agtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgg

SEQ ID NO: 53 - DNA, 56 bp, Oryctolagus cuniculus
  source 1..56: mol_type = unassigned DNA
ctcctgggcaacgtgctggttgttgtgctgtctcatcattttggcaaagaattcac

SEQ ID NO: 54 - DNA, 657 bp, synthetic construct
  source 1..657: mol_type = other DNA
ggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcgggg

SEQ ID NO: 55 - DNA, 861 bp, synthetic construct
  source 1..861: mol_type = other DNA
atgagtattcaacatttcagagtagcccttattcccttttttgctgcattttgccttcctgtttttgctcacccagaaaccctggtgaaagtaaaagatgctgaagatcagttgggtgcaagggtgggttacatagaactggatctcaacagtggtaagatccttgagagttttaggccagaagaaaggtttccaatgatgagcacttttaaagttctgctatgtggagcagtattatccaggattgatgctgggcaagagcaactaggtagaagaatacactattctcagaatgacttggttgagtactcaccagtcacagaaaagcatcttacagatggcatgacagtaagagaattatgcagtgctgccataaccatgagtgataacactgctgccaacttacttctgacaaccattggaggacccaaggagctaacagcttttttgcacaacatgggggatcatgtaactaggcttgataggtgggaaccagagctgaatgaagccataccaaatgatgagagggacaccaccatgcctgtagcaatggcaacaaccttgaggaaactattaactggagaactacttactctagcttccaggcaacaattaatagactggatggaggcagataaagttgcaggaccacttctgagatcagcccttccagctggctggtttattgctgataaatctggagcaggtgagagggggtctaggggtatcattgcagcactggggccagatggtaagccctccagaatagtagttatctacaccacagggagtcaggcaactatggatgaaagaaatagacagatagctgagataggtgcctcactgattaagcattggtaa

SEQ ID NO: 56 - DNA, 735 bp, synthetic construct
  source 1..735: mol_type = other DNA
atggtgagcaagggcgaggagctgatcaaggagaacatgcacatgaagctgtacatggaaggcaccgtgaacaaccaccacttcaagtgcaccaccgaaggggagggcaagccctacgagggcacccagacccagaggattaaggtggtggagggaggccccctgccgttcgcattcgacatcctggccacctgctttatgtacgggagcaagaccttcatcaaccacacccagggcatccccgatttctttaagcagtccttccctgagggcttcacatgggagagagtcaccacatacgaagacgggggcgtgcttaccgttacccaggacaccagcctccaggacggctgcttgatctacaacgtcaagctcagaggggtgaacttcccatccaacggccctgtgatgcagaagaaaacactcggctgggaggccaccaccgagaccctgtaccccgctgacggcggcctggaaggcagatgcgacatggccctgaagctcgtgggcgggggccacctgcactgcaacctgaagaccacatacagatccaagaaacccgctaagaacctcaagatgcccggcgtctactttgtggaccgcagactggaaagaatcaaggaggccgacaatgagacctacgtcgagcagcacgaggtggctgtggccagatactgcgacctccctagcaaactggggcacaaacttaatggcatggacgagctgtacaagtaa

SEQ ID NO: 57 - DNA, 732 bp, synthetic construct
  source 1..732: mol_type = other DNA
gtcagcaaaggcgaggagctcatcaaagagaacatgcacatgaaactctacatggaaggcaccgtcaacaaccaccacttcaaatgcaccaccgaaggggagggcaaaccctacgagggcacccagacccagagaattaaagtagtagagggaggccccctcccgttcgcattcgacatcctagccacctgctttatgtacgggagcaaaaccttcatcaaccacacccagggcatccccgatttctttaaacagtccttccctgagggcttcacatgggagagagtcaccacatacgaagacgggggcgtcctaaccgttacccaggacaccagcctccaggacggctgcttaatctacaacgtcaaactcagaggggtcaacttcccatccaacggccctgtcatgcagaaaaaaacactcggctgggaggccaccaccgagaccctgtaccccgctgacggcggcctagaaggcagatgcgacatggccctcaaactcgtaggcgggggccacctacactgcaacctaaaaaccacatacagatccaaaaaacccgctaaaaacctaaaaatgcccggcgtctactttgtagaccgcagactagaaagaatcaaagaggccgacaatgagacctacgtagagcagcacgaggtagctgtagccagatactgcgacctccctagcaaactagggcacaaacttaatggcatggacgagctctacaaataa

SEQ ID NO: 58 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 59 - DNA, 717 bp, synthetic construct
  source 1..717: mol_type = other DNA
gtaagcaaaggcgaggagctattcaccggggtagtacccatcctagtcgagctagacggcgacgtaaacggccacaaattcagcgtatccggcgagggcgagggcgatgccacctacggcaaactaaccctaaaattcatctgcaccaccggcaaactacccgtaccctggcccaccctcgtaaccaccctaacctacggcgtacagtgcttcagccgctaccccgaccacatgaaacagcacgacttcttcaaatccgccatgcccgaaggctacgtccaggagcgcaccatcttcttcaaagacgacggcaactacaaaacccgcgccgaggtcaaattcgagggcgacaccctagtcaaccgcatcgagctaaaaggcatcgacttcaaagaggacggcaacatcctagggcacaaactagagtacaactacaacagccacaacgtctatatcatggccgacaaacagaaaaacggcatcaaagtcaacttcaaaatccgccacaacatcgaggacggcagcgtacagctcgccgaccactaccagcagaacacccccatcggcgacggccccgtactactacccgacaaccactacctaagcacccagtccgccctaagcaaagaccccaacgagaaacgcgatcacatggtcctactagagttcgttaccgccgccgggatcactctcggcatggacgagctatacaaataa

SEQ ID NO: 60 - DNA, 561 bp, synthetic construct
  source 1..561: mol_type = other DNA
atgaccagtagatgcatcctccaaacaactctcctgttgtatttctccaccatggctcttgccatgagcaatgacttgcttagatcccagctaagcagcagcagtttggagtgtcaggagctcctattacagttgaatggaaccactgaatattgcctcaaggacaggataaactttgagatccctgaggaaattgagaaatcaagacagttccagaaggaggacatcatattgatcacccatgagatgttccagaagatctttgatattttcaggagaaatatctctagaacaggatggaatgagaccactgttgagaaccttcttgtgaagctccactggcagaaggaacatctggagataatcctggaggatgtcaaagagaaggaaaacttcacctgggacaacaggactcttctgcacctgaagaaatattacttaaggattgtgcagtacctgaaggccaaggagtacagcatctgtgcctggacaatagtccaagcagaaatctgcaggaactttttcttccttaatatacttacagattatctccagaactga

SEQ ID NO: 61 - protein, 184 aa, Rattus sp.
MANRWTLHIAFLLCFSTTALSIDYKQLQFRQSTSIRTCQKLLRQLNGRLNLSYRTDFKIPMEVMHPSQMEKSYTAFAIQVMLQNVFLVFRSNFSSTGWNETIVESLLDELHQQTELLEIILKEKQEERLTWVTSTTTLGLKSYYWRVQRYLKDKKYNSYAWMVVRAEVFRNFSIILRLNRNFQN

SEQ ID NO: 62 - DNA, 555 bp, Rattus sp.
  source 1..555: mol_type = unassigned DNA
atggccaacaggtggaccctccacattgcgttcctgctgtgcttctccaccactgccctctccatcgactacaagcagctccagttccgacaaagcactagcattcggacatgtcagaagctcctgaggcagctgaatggaaggctcaacctcagctacaggacggacttcaagatccctatggaggtgatgcacccgtcacagatggagaagagttacactgcctttgccattcaagtgatgctccagaatgtctttcttgtcttcagaagcaatttctccagcactgggtggaatgagactattgttgaaagtctcttggatgaactacatcagcagacagagcttctggagataatactaaaggaaaagcaagaggaaagattgacttgggtgacatccacgactactttaggcttgaagagctattactggagggtacaaaggtaccttaaagacaagaagtacaacagctatgcctggatggtggtccgagcagaagtcttcaggaacttttccattattctaagacttaatagaaacttccagaactga

SEQ ID NO: 63 - DNA, 555 bp, synthetic construct
  source 1..555: mol_type = other DNA
atggccaacaggtggaccctccacattgccttcctgctgtgcttctccaccactgccctctccattgactacaagcagctccagttcagacaaagcactagcattaggacatgtcagaagctcctgaggcagctgaatggaaggctcaacctcagctacaggactgacttcaagatccctatggaggtgatgcacccatcacagatggagaagagttacactgcctttgccattcaagtgatgctccagaatgtctttcttgtcttcagaagcaatttctccagcactgggtggaatgagactattgttgaaagtctcttggatgaactacatcagcagacagagcttctggagataatactaaaggaaaagcaagaggaaagattgacttgggtgacatccactactactttaggcttgaagagctattactggagggtacaaaggtaccttaaagacaagaagtacaacagctatgcctggatggtggtcagagcagaagtcttcaggaacttttccattattctaagacttaatagaaacttccagaactga

SEQ ID NO: 64 - DNA, 991 bp, synthetic construct; identical to SEQ ID NO: 48
  source 1..991: mol_type = other DNA

SEQ ID NO: 65 - DNA, 1648 bp, synthetic construct; identical to SEQ ID NO: 50
  source 1..1648: mol_type = other DNA

SEQ ID NO: 66 - DNA, 1653 bp, synthetic construct
  source 1..1653: mol_type = other DNA
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 67 - DNA, 1650 bp, synthetic construct
  source 1..1650: mol_type = other DNA
gaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 68 - DNA, 1650 bp, synthetic construct
  source 1..1650: mol_type = other DNA
gaagatgccaaaaacattaaaaaaggcccagcgccattctacccactcgaagacgggaccgccggcgagcagctacacaaagccatgaaacgctacgccctagtacccggcaccatcgcctttaccgacgcacatatcgaggtagacattacctacgccgagtacttcgagatgagcgttcggctagcagaagctatgaaacgctatgggctcaatacaaaccatcggatcgtagtctgcagcgagaatagcttacagttcttcatgcccgtcttaggtgccctcttcatcggtgttgctgttgccccagctaacgacatctacaacgagcgcgagctactcaacagcatgggcatcagccagcccaccgtcgtattcgtcagcaaaaaagggctccaaaaaatcctcaacgtccaaaaaaaactaccgatcatacaaaaaatcatcatcatggatagcaaaaccgactaccagggcttccaaagcatgtacaccttcgtcacttcccatttaccacccggcttcaacgagtacgacttcgttcccgagagcttcgaccgggacaaaaccatcgccctcatcatgaacagtagtggcagtaccggattacccaaaggcgtagccctaccgcaccgcaccgcttgtgtccgattcagtcatgcccgcgaccccatcttcggcaaccagatcatccccgacaccgctatcctcagcgtagttccatttcaccacggcttcggcatgttcaccactctaggctacttaatctgcggctttcgggtcgtcctcatgtaccgcttcgaggaggagctattcttacgcagcttacaagactataaaattcaatctgccctcctcgttcccacactatttagcttcttcgctaaaagcactctcatcgacaaatacgacctaagcaacttacacgagatcgccagcggcggggcgccgctcagcaaagaggtaggtgaggccgttgccaaacgcttccacctaccaggcatccgccagggctacggcctcacagaaacaaccagcgccattctcatcacccccgaaggggacgacaaacctggcgcagtaggcaaagtcgttcccttcttcgaggctaaagtcgttgacttagacaccggtaaaacactaggtgtcaaccagcgcggcgagctctgcgtccgtggccccatgatcatgagcggctacgttaacaaccccgaggctacaaacgctctcatcgacaaagacggctggctacacagcggcgacatcgcctactgggacgaggacgagcacttcttcatcgttgaccggctaaaaagcctcatcaaatacaaaggctaccaggtagccccagccgaactagagagcatcctcctccaacaccccaacatcttcgacgccggggtcgccggcctacccgacgacgatgccggcgagctacccgccgcagtcgtcgttctagaacacggtaaaaccatgaccgagaaagagatcgttgactatgttgccagccaggttacaaccgccaaaaaactacgcggtggtgttgtcttcgttgacgaggttcctaaaggactaaccggcaagttagacgcccgcaaaatccgcgagattctcattaaagccaaaaaaggcggcaaaatcgccgtctaa

SEQ ID NO: 69 - DNA, 513 bp, synthetic construct
  source 1..513: mol_type = other DNA
gtcttcacactcgaagatttcgttggggactggcgacagacagccggctacaacctagaccaagtccttgaacagggaggtgtctccagtttatttcagaatctcggggtctccgtaactccgatccaaagaattgtcctcagcggtgaaaatgggctcaaaatcgacatccatgtcatcatcccgtatgaaggtctcagcggcgaccaaatgggccagatcgaaaaaatttttaaagtagtctaccctgtagatgatcatcactttaaagtaatcctccactatggcacactagtaatcgacggggttaccccgaacatgatcgactatttcggacggccgtatgaaggcatcgccgtcttcgacggcaaaaaaatcactgtaacagggaccctctggaacggcaacaaaattatcgacgagcgcctcatcaaccccgacggctccctcctcttccgagtaaccatcaacggagtcaccggctggcggctatgcgaacgcattctagcgtaa

SEQ ID NO: 70 - DNA, 843 bp, synthetic construct
  source 1..843: mol_type = other DNA
gtcagcaaaggcgaggagctcttcaccggggtagtccccatcctagtcgagctagacggcgacgtaaacggccacaaattcagcgtctccggcgagggcgagggcgatgccacctacggcaaactcaccctcaaattcatctgcaccaccggcaaactccccgtcccctggcccaccctcgtcaccaccctcacctacggcgtccagtgcttcagccgctaccccgaccacatgaaacagcacgacttcttcaaatccgccatgcccgaaggctacgtccaggagcgcaccatcttcttcaaagacgacggcaactacaaaacccgcgccgaggtcaaattcgagggcgacaccctagtcaaccgcatcgagctcaaaggcatcgacttcaaagaggacggcaacatcctagggcacaaactagagtacaactacaacagccacaacgtctatatcatggccgacaaacagaaaaacggcatcaaagtcaacttcaaaatccgccacaacatcgaggacggcagcgtccagctcgccgaccactaccagcagaacacccccatcggcgacggccccgtcctcctccccgacaaccactacctcagcacccagtccgccctcagcaaagaccccaacgagaaacgcgatcacatggtcctcctagagttcgtcaccgccgccgggatcactctcggcatggacgagctctacaaaaaacttagccatggcttcccgccggaggtagaggagcaggatgatggcacgctccccatgtcttgtgcccaggagagcgggatggaccgtcaccctgcagcctgtgcttctgctagaatcaatgtctag

SEQ ID NO: 71 - protein, 189 aa, Cavia porcellus
MASWYILQIALLLSFTTSLSLSYDLLQLQQQQQRSSLACLKLLRQVKGKPENCHQDRIDFKFPEEILQPQQFQKEKAALVIQEMLQNVLGIFRKNISTTVWNETIVENLTDELHQQMDHLKSTILQERLEDKNMTMRDTMTTLRLKSYYWRISKYLRAKKNSICAWTVVQAELIRNFSIINRLTNYFQD

SEQ ID NO: 72 - DNA, 570 bp, Cavia porcellus
  source 1..570: mol_type = unassigned DNA
atggccagctggtatatcctccaaatcgctctcctgctgtccttcaccacatctctttccttaagctacgacttgcttcaactccagcaacaacaacaacgcagcagtttggcctgtctgaaacttctgaggcaggtgaaaggaaagcctgaaaactgccaccaggacaggatcgatttcaagtttccagaagagatcttgcaaccacagcagttccagaaggagaaggctgctcttgtcatccaagagatgcttcagaacgtcttgggtattttcagaaaaaacatttccaccactgtatggaatgagaccattgtggagaacctcactgatgaactccatcagcaaatggatcatctgaagtcaacgatcctgcaggaaagactggaggacaaaaacatgaccatgagggataccatgaccactctgcgtttgaagagctattactggaggatcagcaagtacctcagagccaagaagaacagcatttgtgcctggacagtagtccaggcagaactcatcaggaacttttccatcattaacagacttacaaattacttccaagactga